SEQUENCE LISTING (33 sequences)

SEQ ID NO: 1 - protein, 432 aa, Homo sapiens
  SITE 105: note = Phosphorylated residue
  SITE 163: note = Phosphorylated residue
  SITE 181: note = Phosphorylated residue
  SITE 227: note = Phosphorylated residue
  SITE 317: note = Phosphorylated residue
  SITE 341: note = Phosphorylated residue
  SITE 359: note = Phosphorylated residue
  SITE 387: note = Phosphorylated residue
  SITE 417: note = Phosphorylated residue
MGPAGSLLGSGQMQITLWGSLAAVAIFFVITFLIFLCSSCDREKKPRQHSGDHENLMNVPSDKEMFSRSVTSLATDAPASSEQNGALTNGDILSEDSTLTCMQHYEEVQTSASDLLDSQDSTGKPKCHQSRELPRIPPESAVDTMLTARSVDGDQGLGMEGPYEVLKDSSSQENMVEDCLYETVKEIKEVAAAAHLEKGHSGKAKSTSASKELPGPQTEGKAEFAEYASVDRNKKCRQSVNVESILGNSCDPEEEAPPPVPVKLLDENENLQEKEGGEAEESATDTTSETNKRFSSLSYKSREEDPTLTEEEISAMYSSVNKPGQLVNKSGQSLTVPESTYTSIQGDPQRSPSSCNDLYATVKDFEKTPNSTLPPAGRPSEEPEPDYEAIQTLNREEEKATLGTNGHHGLVPKENDYESISDLQQGRDITRL

SEQ ID NO: 2 - protein, 16 aa, Homo sapiens
MGPAGSLLGSGQMQIT

SEQ ID NO: 3 - protein, 17 aa, Mus musculus
MGPAGSVLSSGQMQMQM

SEQ ID NO: 4 - protein, 21 aa, Homo sapiens
LWGSLAAVAIFFVITFLIFLC

SEQ ID NO: 5 - protein, 118 aa, synthetic construct
EVQLQQSGPELVKPGASVKMSCKASGYTFTSYVMHWVKQKPGQGLEWIGYIYPYNDGTKYNEKFKGKATLTSDKSSSTAYMELSSLTSEDSAVYYCARYKYGQGFAYWGQGTRVTVSA

SEQ ID NO: 6 - protein, 8 aa, synthetic construct
GYTFTSYV

SEQ ID NO: 7 - protein, 8 aa, synthetic construct
IYPYNDGT

SEQ ID NO: 8 - protein, 11 aa, synthetic construct
ARYKYGQGFAY

SEQ ID NO: 9 - protein, 19 aa, synthetic construct
MGWSCIILFLVATATGVHS

SEQ ID NO: 10 - protein, 442 aa, synthetic construct
EVQLQQSGPELVKPGASVKMSCKASGYTFTSYVMHWVKQKPGQGLEWIGYIYPYNDGTKYNEKFKGKATLTSDKSSSTAYMELSSLTSEDSAVYYCARYKYGQGFAYWGQGTRVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 11 - protein, 107 aa, synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIK

SEQ ID NO: 12 - protein, 6 aa, synthetic construct
ENIYSN

SEQ ID NO: 13 - skipped

SEQ ID NO: 14 - protein, 9 aa, synthetic construct
QHFWGTPWT

SEQ ID NO: 15 - protein, 214 aa, synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 114 aa, synthetic construct
QIQLVQSGPELKKPGETVKISCKASGYIFTNYGMNWVKQAPGKGLKWMGWINPYTGEATYDDDFKGRFAFSLETSANTAYLQINNLKNEDTATYFCAKTGTTYWGQGTLVTVSA

SEQ ID NO: 17 - protein, 8 aa, synthetic construct
GYIFTNYG

SEQ ID NO: 18 - protein, 8 aa, synthetic construct
INPYTGEA

SEQ ID NO: 19 - protein, 7 aa, synthetic construct
AKTGTTY

SEQ ID NO: 20 - protein, 438 aa, synthetic construct
QIQLVQSGPELKKPGETVKISCKASGYIFTNYGMNWVKQAPGKGLKWMGWINPYTGEATYDDDFKGRFAFSLETSANTAYLQINNLKNEDTATYFCAKTGTTYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 21 - protein, 106 aa, synthetic construct
QIVLTQSPAIMSASPGEKVTMTCSASSSVSYMYWYQQKSGTSPKRWIYDTSKLASGVPARFSGSGSGTSYSLTINNMEAEDAATYYCQQWSSNPLTFGAGTKLELK

SEQ ID NO: 22 - protein, 5 aa, synthetic construct
SSVSY

SEQ ID NO: 23 - skipped

SEQ ID NO: 24 - protein, 9 aa, synthetic construct
QQWSSNPLT

SEQ ID NO: 25 - protein, 213 aa, synthetic construct
QIVLTQSPAIMSASPGEKVTMTCSASSSVSYMYWYQQKSGTSPKRWIYDTSKLASGVPARFSGSGSGTSYSLTINNMEAEDAATYYCQQWSSNPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 26 - protein, 15 aa, synthetic construct
GGGGSGGGGSGGGGS

SEQ ID NO: 27 - protein, 118 aa, synthetic construct
QVQLVQSGAEVKKPGASVKVSCKASGYTFTSYVMHWVRQAPGQGLEWMGYIYPYNDGTKYNEKFKGRVTMTRDTSTSTVYMELSSLRSEDTAVYYCARYKYGQGFAYWGQGTTVTVSS

SEQ ID NO: 28 - protein, 117 aa, synthetic construct
DIQMTQSPSSVSASVGDRVTITCRASENIYSNLAWYQQKPGKAPKLLIYAATNLADGVPSRFSGSGSGTDFTLTISSLQPEDFATYYCQHFWGTPWTFGGGTKLEIKRLEPKSCDKT

SEQ ID NO: 29 - DNA, 354 bp, synthetic construct
  source 1..354: mol_type = other DNA
gaggtccagctgcagcagtctggacctgaactggtaaagcctggggcttcagtgaagatgtcctgcaaggcttctggatacacattcactagctatgttatgcactgggtgaagcagaagcctgggcagggccttgagtggattggatatatttatccttacaatgatggtactaagtacaatgagaagttcaaaggcaaggccacactgacttcagacaaatcctccagcacagcctacatggagctcagcagcctgacctctgaggactctgcggtctattattgtgcaagatataagtatggtcaggggtttgcttactggggccaagggactcgggtcactgtctctgca

SEQ ID NO: 30 - DNA, 321 bp, synthetic construct
  source 1..321: mol_type = other DNA
gacatccagatgactcagtctccagcctccctatctgtatctgtgggagaaactgtcaccatcacatgtcgagcaagtgagaatatttacagtaatttagcatggtatcagcagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggagttattactgtcaacatttttggggtactccgtggacgttcggtggaggcaccaagctggaaatcaaa

SEQ ID NO: 31 - DNA, 342 bp, synthetic construct
  source 1..342: mol_type = other DNA
cagatccagttggtgcagtctggacctgagctgaagaagcctggagagacagtcaagatctcctgcaaggcttctgggtatatcttcacaaactatggaatgaactgggtgaagcaggctccaggaaagggtttaaagtggatgggctggataaacccctacactggagaggcaacatatgatgatgacttcaagggacggtttgccttctctttggaaacctctgccaacactgcctatttgcagatcaacaacctcaaaaatgaggacacggctacatatttctgtgcaaagactgggacgacttactggggccaagggactctggtcactgtctctgca

SEQ ID NO: 32 - DNA, 318 bp, synthetic construct
  source 1..318: mol_type = other DNA
caaattgttctcacccagtctccagcaatcatgtctgcatctccaggggagaaggtcaccatgacctgcagtgccagctcaagtgtaagttacatgtactggtaccagcagaagtcaggcacctcccccaaaagatggatttatgacacatccaaactggcttctggagtccctgctcgcttcagtggcagtgggtctgggacctcttactctctcacaatcaacaacatggaggctgaagatgctgccacttattactgccagcagtggagtagtaacccgctcacgttcggtgctgggaccaagctggagctgaaa

SEQ ID NO: 33 - protein, 108 aa, synthetic construct
DIQMTQSPSSVSASVGDRVTITCRASENIYSNLAWYQQKPGKAPKLLIYAATNLADGVPSRFSGSGSGTDFTLTISSLQPEDFATYYCQHFWGTPWTFGGGTKLEIKR

(19) United States
(12) Patent Application Publication
(10) Pub. No.: US 2025/0263483 A1
(43) Pub. Date: Aug. 21, 2025

(54) ANTI-PAG ANTIBODIES AND THEIR USE TO TREAT CANCER AND LIMIT TUMOR GROWTH

(71) Applicant: The Trustees of Columbia University in the City of New York, New York, NY (US)

(72) Inventors: Adam MOR, Old Tappan, NJ (US); Marianne STRAZZA, New York, NY (US)

(21) Appl. No.: 19/197,534

(22) Filed: May 2, 2025

Related U.S. Application Data
(63) Continuation-in-part of application No. PCT/US23/78744, filed on Nov. 3, 2023.
(60) Provisional application No. 63/382,483, filed on Nov. 4, 2022.

(51) Int. Cl. C07K 16/28; A61K 39/00; A61P 35/00

(52) U.S. Cl. CPC C07K 16/28; A61P 35/00; C07K 16/2818; A61K 2039/507; C07K 2317/52; C07K 2317/622; C07K 2317/76

(57) ABSTRACT

The subject matter described here relates to anti-PAG antibodies. The subject matter described here also provides a pharmaceutical composition of, or a kit for generating an anti-PAG antibody; a method of making an anti-PAG antibody; a method for treating or preventing cancer with an anti-PAG antibody optionally in combination with an anti-PD-1 antibody; and one or more host cells comprising a polypeptide-encoding an anti-PAG antibody.

This application is a continuation-in-part of International Patent Application PCT/US2023/078744, filed Nov. 3, 2023 which claims the benefit of and priority to U.S. Application No. 63/382,483, filed Nov. 4, 2022, entitled “NEUTRALIZATION OF THE ADAPTOR PROTEIN PAG BY MONOCLONAL ANTIBODY LIMITS TUMOR GROWTH”, the contents of which are hereby incorporated by reference in their entireties.

### GOVERNMENT SUPPORT

This invention was made with government support under AI125640, and AI150597 awarded by the National Institutes of Health. The government has certain rights in the invention.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entireties.

### SEQUENCE LISTING

The instant application contains a Sequence Listing which has been submitted electronically in XML format and is hereby incorporated by reference in its entirety. Said XML copy, created on Apr. 29, 2025, is named 0019240_01298US2_SL.xml and is 35,252 bytes in size.

### TECHNICAL FIELD

The present invention relates generally to the treatment or prevention of cancer with antibodies. More particularly, the present invention relates to anti-PAG antibodies capable of impacting tumor size.

### BACKGROUND

Immune checkpoint therapy is a relatively new modality in the treatment of cancer. Specifically, PD-1 and PD-L1 targeting antibodies release the brakes on a patient's T-cells allowing for a more robust anti-tumor immune response. Despite great promise for success, the average response rate to PD-1 binding blockade for most tumors is low leaving open the opportunity for improvement. There is still a need for prophylactic and/or therapeutic agents with improved efficacy.

Phosphorylatyion of the transmembrane adaptor phosphoprotein associated with glycosphingolipid-enriched microdomains 1 (PAG), a member of the transmembrane adaptor protein (TRAP) family, is associated with the inhibition of various T-cell functions downstream of PD-1. Furthermore, PAG expression is negatively correlated with survival in multiple human tumors.

### SUMMARY

It is understood that any of the embodiments described below can be combined in any desired way, and that any embodiment or combination of embodiments can be applied to each of the aspects described below, unless the context indicates otherwise. In certain aspects, the present disclosure provides a monoclonal antibody or a fragment thereof, comprising: a first arm comprising a first variable heavy chain domain and a first variable light chain domain, wherein a portion of the first arm is capable of binding to an extracellular portion of human PAG; and a second arm comprising a second variable heavy chain domain and a second variable light chain domain, wherein a portion of the second arm is capable of binding to an extracellular portion of human PAG. In some embodiments, the first and second arms each further comprise a fragment, crystallizable (Fc) domain.

In some embodiments, the first and second arms of the monoclonal antibody each further comprise a CH1 domain, a hinge domain, and a CL domain.

In some embodiments, the first variable heavy chain domain of the first arm of the monoclonal antibody is encoded by a first polypeptide chain. In some embodiments, the first variable light chain domain of the first arm of the monoclonal antibody is encoded by a second polypeptide chain. In some embodiments, the second variable heavy chain domain of the second arm of the monoclonal antibody is encoded by a third polypeptide chain. In some embodiments, the second variable light chain domain of the second arm of the monoclonal antibody is encoded by a fourth polypeptide chain. In some embodiments, the first variable heavy chain domain and first variable light chain domain of the monoclonal antibody form a first PAG binding site. In some embodiments, the second variable heavy chain domain and second variable light chain domain of the monoclonal antibody form a second PAG binding site. In some embodiments, the first and second PAG binding sites are the same. In some embodiments, the first and third polypeptide chain each further encode a hinge domain, a CH1 domain, and the Fc domain, and wherein the second and fourth polypeptide chain each further encode a CL domain. In some embodiments, the first and third polypeptide chains comprise the same sequence and the second and fourth polypeptide chains comprise the same sequence. In some embodiments, the first and third polypeptide chain each comprises an amino acid sequence comprising SEQ ID NO: 5, 10, 16, 20, or 27 and the second and fourth polypeptide chain each comprises an amino acid sequence comprising SEQ ID NO: 11, 15, 21, 25, 28, or 33.

In some embodiments, the first variable heavy chain domain comprises an amino acid sequence comprising SEQ ID NO: 5, 16, or 27, and the first variable light chain domain comprises an amino acid sequence comprising SEQ ID NO: 11, 21, 28, or 33.

In some embodiments, the first arm comprises an amino acid sequence comprising SEQ ID NO: 5 and the second arm comprises an amino acid sequence comprising SEQ ID NO: 11, the first arm comprises an amino acid sequence comprising SEQ ID NO: 10 and the second arm comprises an amino acid sequence comprising SEQ ID NO: 15, the first arm comprises an amino acid sequence comprising SEQ ID NO: 16 and the second arm comprises an amino acid sequence comprising SEQ ID NO: 21, the first arm comprises an amino acid sequence comprising SEQ ID NO: 20 and the second arm comprises an amino acid sequence comprising SEQ ID NO: 25, the first arm comprises an amino acid sequence comprising SEQ ID NO: 27 and the second arm comprises an amino acid sequence comprising SEQ ID NO: 28, or the first arm comprises an amino acid sequence comprising SEQ ID NO: 27 and the second arm comprises an amino acid sequence comprising SEQ ID NO: 33.

In some embodiments, the first and second variable heavy chain domains each comprises a CDR-H1, CDR-H2, and CDR-H3 domain, and the first and second variable light chain domains each comprises a CDR-L1, CDR-L2, and CDR-L3 domain. In some embodiments, the CDR-H1, CDR-H2, and CDR-H3 of the first heavy chain domains and the CDR-H1, CDR-H2, and CDR-H3 of the second heavy chain domains comprises an amino acid sequence comprising SEQ ID NO: 6, 7, and 8, respectively, and wherein the CDR-L1, CDR-L2, and CDR-L3 of the first light chain domains and the CDR-L1, CDR-L2, and CDR-L3 of the second light chain domains comprises an amino acid sequence comprising SEQ ID NO: 12, 13, and 14, respectively.

In some embodiments, first and second polypeptide chains are linked by one or more covalent disulfide bonds and the third and fourth polypeptide chains are linked by one or more covalent disulfide bonds. In some embodiments, the first and third polypeptide chains are linked by one or more covalent disulfide bonds.

In certain aspects, the present disclosure provides a scFv comprising a polypeptide comprising a variable heavy chain domain and a variable light chain domain. In some embodiments, the variable heavy chain domain and variable light chain domain form a binding site to an extracellular portion of human PAG.

In some embodiments, the scFv further comprises a linker between the variable heavy chain domain and a variable light chain domain. In some embodiments, the linker is a glycine serine linker. In some embodiments, the linker is a glycine serine linker about 15 amino acids in length. In some embodiments, the linker comprises SEQ ID NO: 26. In some embodiments, the polypeptide further comprises an Fc domain. In some embodiments, the variable heavy chain domain comprises an amino acid sequence comprising SEQ ID NO: 5, 16, or 27, and the variable light chain domain comprises an amino acid sequence comprising SEQ ID NO: 11, 21, 28, or 33. In some embodiments, the variable heavy chain domain comprises an amino acid sequence comprising SEQ ID NO: 5 and the variable light chain domain comprises an amino acid sequence comprising SEQ ID NO: 11, the variable heavy chain domain comprises an amino acid sequence comprising SEQ ID NO: 16 and the variable light chain domain comprises an amino acid sequence comprising SEQ ID NO: 21, the variable heavy chain domain comprises an amino acid sequence comprising SEQ ID NO: 27 and the variable light chain domain comprises an amino acid sequence comprising SEQ ID NO: 28, or the variable heavy chain domain comprises an amino acid sequence comprising SEQ ID NO: 27 and the variable light chain domain comprises an amino acid sequence comprising SEQ ID NO: 33.

In some embodiments, the variable heavy chain domain comprises a CDR-H1, CDR-H2, and CDR-H3 domain, and the variable light chain domain comprises a CDR-L1, CDR-L2, and CDR-L3 domain. In some embodiments, the CDR-H1, CDR-H2, and CDR-H3 of the heavy chain domain comprises an amino acid sequence comprising SEQ ID NO: 6, 7, and 8, respectively, and wherein the CDR-L1, CDR-L2, and CDR-L3 of the light chain domain comprises an amino acid sequence comprising SEQ ID NO: 12, 13, and 14, respectively.

In some embodiments, the polypeptide further comprises a second variable heavy chain domain and a second variable light chain domain. In some embodiments, the second variable heavy chain domain and second variable light chain domain form a second binding site to an extracellular portion of human PAG. In some embodiments, the scFv further comprises a linker between the second variable heavy chain domain and the second variable light chain domain. In some embodiments, the linker is a glycine serine linker. In some embodiments, the linker is a glycine serine linker about 15 amino acids in length. In some embodiments, the linker comprises SEQ ID NO: 26. In some embodiments, the first and second PAG binding sites are the same. In some embodiments, the first and second variable heavy chain domains comprise the same sequence and the first and second variable light chain domains comprise the same sequence. In some embodiments, the first and second variable heavy chain domains each comprises an amino acid sequence comprising SEQ ID NO: 5, 16, or 27, and wherein the first and second variable light chain domains each comprise an amino acid sequence comprising SEQ ID NO: 11, 21, 28, or 33. In some embodiments, the first and second variable heavy chain domains each comprise an amino acid sequence comprising SEQ ID NO: 5 and the first and second variable light chain domains each comprise an amino acid sequence comprising SEQ ID NO: 11, the first and second variable heavy chain domains each comprise an amino acid sequence comprising SEQ ID NO: 16 and the first and second variable light chain domains each comprise an amino acid sequence comprising SEQ ID NO: 21, the first and second variable heavy chain domains each comprise an amino acid sequence comprising SEQ ID NO: 27 and the first and second variable light chain domains each comprise an amino acid sequence comprising SEQ ID NO: 28, or the first and second variable heavy chain domains each comprise an amino acid sequence comprising SEQ ID NO: 27 and the first and second variable light chain domains each comprise an amino acid sequence comprising SEQ ID NO: 33.

In some embodiments, the first and second variable heavy chain domains each comprises a CDR-H1, CDR-H2, and CDR-H3 domain, and the first and second variable light chain domains each comprises a CDR-L1, CDR-L2, and CDR-L3 domain. In some embodiments, the CDR-H1, CDR-H2, and CDR-H3 of the first heavy chain domains and the CDR-H1, CDR-H2, and CDR-H3 of the second heavy chain domains comprises an amino acid sequence comprising SEQ ID NO: 6, 7, and 8, respectively. In some embodiments, the CDR-L1, CDR-L2, and CDR-L3 of the first light chain domains and the CDR-L1, CDR-L2, and CDR-L3 of the second light chain domains comprises an amino acid sequence comprising SEQ ID NO: 12, 13, and 14, respectively.

In some embodiments, the Fc domain of the polypeptide associates with the Fc domain of a second polypeptide, wherein the second polypeptide is identical to the first polypeptide. In some embodiments, the Fc region of the first polypeptide comprises knob mutations and the Fc region of the second polypeptide comprise hole mutations, or vice versa.

In some embodiments, the monoclonal antibody or scFv is capable of localizing a PAG protein away from an immune synapse.

In some embodiments, the monoclonal antibody or scFv is capable of localizing a PD-1 protein away from an immune synapse.

In some embodiments, the monoclonal antibody or scFv is capable of disrupting downstream signaling of a PD-1 mediated response in a T cell.

In some embodiments, the monoclonal antibody or scFv is capable of disrupting downstream signaling of a PAG mediated response in a T cell.

In some embodiments, the monoclonal antibody or scFv is capable of enhancing T cell function.

In some embodiments, the PAG protein is located on a T cell, and the monoclonal antibody or scFv is capable of preventing the phosphorylation of PAG protein downstream of PD-1 signaling.

In some embodiments, the monoclonal antibody or scFv is capable of inducing a cytokine secretion in a T cell. In some embodiments, the cytokine secretion is a secretion of IL-2.

In certain aspects, the present disclosure provides a pharmaceutical composition comprising: the monoclonal antibody or the scFv described herein; and a pharmaceutically acceptable carrier.

In certain aspects, the present disclosure provides a method of preventing or treating cancer in a subject comprising administering to the subject an effective amount of any of the pharmaceutical compositions described herein, for example, a pharmaceutical composition comprising: the monoclonal antibody or the scFv described herein; and a pharmaceutically acceptable carrier.

In some embodiments, the cancer is selected from colorectal cancer, lung cancer, bladder cancer, breast cancer, cervical cancer, kidney cancer, leukemia, Hodgkin lymphoma, non-Hodgkin lymphoma, prostate cancer, skin cancer (e.g., melanoma), head and neck cancer, endometrial cancer, colon cancer, rectal cancer, liver cancer, thyroids cancer, esophageal cancer, renal cell cancer, testicular cancers, and a combination thereof.

In some embodiments, the cancer is selected from colorectal cancer, colon adenocarcinoma, renal cell carcinoma, melanoma, acute myeloid leukemia, invasive breast cancer, cervical squamous cancer, and testicular cancer. In some embodiments, the cancer is colorectal cancer. In some embodiments, the cancer is melanoma.

In some embodiments, the administration of the pharmaceutical composition is capable of inhibiting tumor growth. In some embodiments, the administration of the pharmaceutical composition is capable of increasing T cell infiltration in the tumor.

In some embodiments, the pharmaceutical composition is administered in combination with an immune checkpoint therapy. In some embodiments, the immune checkpoint therapy is an anti-PD-1 antibody. In some embodiments, the administration of the pharmaceutical composition in combination with the anti-PD-1 antibody enhances the anti-PD-1 response.

In certain aspects, the present disclosure provides a kit for generating a monoclonal antibody or fragment thereof or an scFv, the kit comprising one or more vectors comprising a polynucleotide sequence encoding any of the monoclonal antibodies or any of the scFvs described herein.

In certain aspects, the present disclosure provides a kit for generating a monoclonal antibody or fragment thereof, the kit comprising: a first vector comprising a polynucleotide sequence encoding the first polypeptide chain of any of the monoclonal antibody described herein; and a second vector comprising a polynucleotide sequence encoding the second polypeptide chain of any of the monoclonal antibody described herein.

In some embodiments, the first vector and the second vector are the same vector. In some embodiments, the first vector and the second vector are two different vectors.

In certain aspects, the present disclosure provides one or more host cells comprising: one or more vectors comprising a polynucleotide sequence encoding any of the monoclonal antibodies or any of the scFvs described herein.

In certain aspects, the present disclosure provides one or more host cells comprising: a first vector comprising a polynucleotide sequence encoding the first polypeptide chain of any of the monoclonal antibody described above; and a second vector comprising a polynucleotide sequence encoding the second polypeptide chain of any of the monoclonal antibody described above.

In some embodiments, the first vector and the second vector are the same vector. In some embodiments, the first vector and the second vector are two different vectors.

In certain aspects, the present disclosure provides a method of making a monoclonal antibody or fragment thereof or scFv comprising: culturing the one or more host cells described herein under conditions suitable for an expression of the one or more vectors; and recovering the monoclonal antibody or fragment thereof or scFv.

In certain aspects, the present disclosure provides a method of making a monoclonal antibody or fragment thereof comprising: culturing the one or more host cells described herein under conditions suitable for an expression of the first vector and the second vector; and recovering the monoclonal antibody or fragment thereof.

In certain aspects, the present disclosure provides a composition comprising: one or more vectors comprising a polynucleotide sequence encoding any of the monoclonal antibodies or any of the scFvs described above.

In certain aspects, the present disclosure provides a composition comprising: a first vector comprising a polynucleotide sequence encoding the first polypeptide chain of any of the monoclonal antibody described herein; and a second vector comprising a polynucleotide sequence encoding the second polypeptide chain of any of the monoclonal antibody. In some embodiments, the first vector and the second vector are the same vector. In some embodiments, the first vector and the second vector are two different vectors.

In certain aspects, the present disclosure provides a means for binding an extracellular portion of a human PAG protein. In some embodiments, the means for binding an extracellular portion of a human PAG protein comprises: a first arm comprising a first variable heavy chain domain and a first variable light chain domain, wherein a portion of the first arm is capable of binding to the extracellular portion of the human PAG protein; and a second arm comprising a second variable heavy chain domain and a second variable light chain domain, wherein a portion of the second arm is capable of binding to the extracellular portion of the human PAG protein, wherein the first and second arms each further comprise a fragment, crystallizable (Fc) domain. In some embodiments, the first and second arms each further comprise a CH1 domain, a hinge domain, and a CL domain.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first PAG binding site and the second variable heavy chain domain and second variable light chain domain form a second PAG binding site. In some embodiments, the first and second PAG binding sites are the same. In some embodiments, the first and third polypeptide chain each further encode a hinge domain, a CH1 domain, and the Fc domain, and the second and fourth polypeptide chain each further encode a CL domain. In some embodiments, the first and third polypeptide chains comprise the same sequence and the second and fourth polypeptide chains comprise the same sequence.

In some embodiments, the first variable heavy chain domain comprises an amino acid sequence of the variable heavy chain portion of SEQ ID NO: 5, 16, or 27, wherein the first variable light chain domain comprises an amino acid sequence of the variable light chain portion of SEQ ID NO: 11, 21, 28, or 33.

In some embodiments, the first arm comprises an amino acid sequence comprising SEQ ID NO: 5 and the second arm comprises an amino acid sequence comprising SEQ ID NO: 11, the first arm comprises an amino acid sequence comprising SEQ ID NO: 10 and the second arm comprises an amino acid sequence comprising SEQ ID NO: 15, the first arm comprises an amino acid sequence comprising SEQ ID NO: 16 and the second arm comprises an amino acid sequence comprising SEQ ID NO: 21, the first arm comprises an amino acid sequence comprising SEQ ID NO: 20 and the second arm comprises an amino acid sequence comprising SEQ ID NO: 25, the first arm comprises an amino acid sequence comprising SEQ ID NO: 27 and the second arm comprises an amino acid sequence comprising SEQ ID NO: 28, or the first arm comprises an amino acid sequence comprising SEQ ID NO: 27 and the second arm comprises an amino acid sequence comprising SEQ ID NO: 33.

In some embodiments, the means comprises any one of the scFvs described herein.

In some embodiments, the means is capable of localizing a PD-1 protein away from an immune synapse. In some embodiments, the means is capable of localizing a PAG protein away from an immune synapse.

In some embodiments, the means is capable of disrupting downstream signaling of a PD-1 mediated response in a T cell.

In some embodiments, the means is capable of disrupting downstream signaling of a PAG mediated response in a T cell. In some embodiments, the means is capable of enhancing T cell function.

In some embodiments, the PAG protein is located on a T cell, and the means is capable of preventing the phosphorylation of PAG protein downstream of PD-1 signaling. In some embodiments, the means is capable of inducing a cytokine secretion in a T cell. In some embodiments, the cytokine secretion is a secretion of IL-2.

In some embodiments, in the methods disclosed herein the subject is a human subject.

### BRIEF DESCRIPTION OF FIGURES

The patent or application file contains at least one drawing executed in color. Copies of this patent or patent application publication with color drawing(s) will be provided by the Office upon request and payment of the necessary fec.

FIGS. 1A-H show the structure and enrichment of PAG at the immune synapse. FIG. 1A PAG-GFP was overexpressed in A549 cells prior to cell lysis and GFP immunoprecipitation. Mass spectrometry (MS) was then performed, and it identified that the extracellular portion of PAG was intact and uncleaved. Underlined text indicates peptides detected by MS, amino acids 17-37 (LWGSLAAVAIFFVITFLIFLC (SEQ ID NO: 4)) that are shaded shows the transmembrane domain, and shaded amino acids with an asterisk specifies phosphorylated tyrosines. FIG. 1A discloses SEQ ID NO: 1. FIG. 1B predicted the structure of the extracellular portion of human and mouse PAG. FIG. 1B discloses SEQ ID NOs: 2 and 3. FIG. 1C shows a Western blot of Jurkat T-cells following plate-bound stimulation. PAG (80 kDa) is phosphorylated PAG based on size; immunoblot PAG (clone MEM-255). FIG. 1D shows the Proximity ligation assay of Jurkat T-cells incubated with control or PD-L2 overexpressing Raji B cells in the presence of superantigen Staphylococcal enterotoxin E (SEE). Cells were permeabilized, and the proximity between endogenous PD-1 and PAG was imaged using intracellular antibodies. Clusters indicate proximity of ≤40 nm. Quantification is done as the number of clusters per cell over 2 independent experiments, with 1,400-2,000 cells counted per condition over the two experiments; graphed points represent fields of cells. ***p≤0.001. FIG. 1E shows a schematic representation of full-length PAG-GFP and Fc-conjugated PAG-GFP. FIG. 1F shows confocal microscopy to assess the location of PAG relative to the immune synapse. Jurkat T-cells were transfected with PAG-GFP or Fc-PAG-GFP and incubated with Raji B cells in the presence of SEE. Cells were imaged by confocal microscopy to assess the location of PAG relative to the immune synapse. FIG. 1G shows the quantification of the number of cells per phenotype. Percentages were counted for 5 independent experiments, ****p≤0.0001. FIG. 1H shows ELISA-measured IL-2 secretion. Jurkat T-cells were transfected with PAG-GFP or Fc-PAG-GFP and incubated with Raji B cells in the presence of SEE, n=2, *p≤0.05.

FIGS. 2A-I show the binding of Clone 7M16A to the extracellular domain of PAG. FIG. 2A shows ELISA with immobilized PAG peptide (human, mouse, or cynomolgus) using hybridoma supernatants. For each hybridoma along the y-axis, the leftmost bar corresponds to the human PAG peptide, the middle bar corresponds to the cynomolgus PAG peptide, and the rightmost bar corresponds to the mouse PAG peptide. FIG. 2B shows 7M16A staining of non-permeabilized wild-type C57BL6 or PAG KO murine splenocytes to show the specificity of binding. FIG. 2C shows a Western blot of A549 cells made to overexpress PAG-GFP stably. FIG. 2D shows a Western blot of fractionated cell lysates showing that PAG-GFP is expressed at high levels at the cell membrane. Confocal imaging showing the localization of PAG-GFP at the plasma membrane. FIG. 2E shows 7M16A staining of non-permeabilized A549 cells showing binding to PAG-GFP on the surface. FIG. 2F shows ELISA with immobilized human PAG peptide using purified monoclonal antibodies. FIG. 2G shows ELISA of IL-2 secretion from primary human CD3⁺ T-cells stimulated as indicated for 48 hours, n=3, *p≤0.05, **p≤0.01.

FIGS. 2H-I show Jurkat T cells stably express PAG-GFP with or without PD-1-SNAP incubated with Raji B cells in the presence of SEE. Cells were imaged by confocal microscopy to assess the location of PAG and PD-1 relative to the immune synapse. The white arrow indicates the center of the immune synapse where PAG was excluded with 7M16A pretreatment. The white star indicates the point in the cell with enrichment of PD-1 either at or away from the immune synapse. Quantification of the number of cells per phenotype. *p≤0.05. For each condition (enriched or excluded) the left-hand bar is “control,” and the right-hand bar is “+7M16A.”

FIGS. 3A-D show PAG antibody and PD-1 antibody in combination limit MC38 tumor growth to a greater extent than the PD-1 antibody alone. MC38 murine colon adenocarcinoma cells were implanted subcutaneously into wild-type C57BL6 mice. Antibody treatment was initiated when tumors were between 60-75 mm³, with dosing every 4 days for a total of 4 doses. FIG. 3A shows tumor volume measured daily by digital caliper. X indicates a deceased mouse prior to the endpoint of the study. Percent of mice with tumors to exceed 500 mm³. The parenthesis shows the number of mice in each treatment group. FIGS. 3B-C show tumor volumes on day 10 (FIG. 3B) and 19 (FIG. 3C) of treatment. FIG. 3D shows the number of days to reach that point for those tumors that exceeded 500 mm³. Unless otherwise noted, all statistical comparisons are to the untreated group. *p≤0.05, **p≤0.01, ***p≤0.001. In FIGS. 3B to 3D, the data is provided in the following order from left to right along the y-axis: Untreated, aPD-1, 7M16A 200 μg, 7M16A 400 μg, 7M16A 200 μg+aPD-1, 7M16A 400 μg+aPD-1.

FIGS. 4A-B show PAG antibody and PD-1 antibody in combination enhance T cell infiltration into MC38 tumors. Immunohistochemistry of dissected tumors at the study endpoint. Sections were stained with anti-CD3 from 2 tumors per condition; representative images were shown (40×) (FIG. 4A). FIG. 4B shows the quantification of CD3⁺ cells per high power field (HPF, 20×) from 2 tumors per condition, *p≤0.05.

FIG. 5 shows a schematic model of PAG exclusion from the immunological synapse using anti-PAG antibodies.

FIG. 6 shows a schematic of the immunization strategy.

FIGS. 7A and B show the ability of the hybridoma supernatants to bind PAG on intact cells. In FIG. 7A, cells were isolated from the spleens of 6-week-old WT or PAG KO C57B16 mice, and 10E5 splenocytes were incubated with clonal supernatants overnight at 4 degrees Celsius and then stained with PE-conjugated goat anti-mouse secondary antibody (ImmunoResearch #115-116-071; 1:100 dilution). In FIG. 7B, A549 cells, either wild-type (WT) or overexpressing PAG-GFP (OE) were stained with the indicated clones and PE-conjugated goat anti-mouse secondary antibody. Experiments were repeated 2-3 independent times; the results are representative.

FIGS. 8A and B show UMAP analysis of CD4+ (FIG. 8A) and CD8+ (FIG. 8B) tumor-infiltrating lymphocytes, demonstrating subset distribution and activation state across the 5 treatment groups. N=3 mice per group.

FIG. 9 shows ELISA binding assays of 7M16A to human PAG-1 peptide.

FIG. 10 shows binding of humanized 7M16A antibodies (7M-hu-1 and 7M-hu-2), D4-hu-1, and a negative control

FIG. 11 shows ELISA binding assay of 7M-hu-1 to human PAG-1 peptide.

### DETAILED DESCRIPTION

All patent applications, published patent applications, issued and granted patents, texts, and literature references cited in this specification are hereby incorporated herein by reference in their entirety to more fully describe the state of the art to which the present disclosed subject matter pertains.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Described herein are specific antibodies for treating cancer (e.g., colon cancer) targeting the PD-1 signaling pathway on T cells. Specific antibodies were identified for targeting PAG in T cells downstream of PD-1 signaling.

The terms “effective amount,” as used herein, refer to an amount or a concentration of one or more compounds or a pharmaceutical composition described herein utilized for a period of time (including in vitro and in vivo acute or chronic administration and periodic or continuous administration) that is effective within the context of its administration for causing an intended effect or physiological outcome.

As used herein, the term “subject” refers to a vertebrate animal. In one embodiment, the subject is a mammal or a mammalian species. In one embodiment, the subject is a human. In one embodiment, the subject is a healthy human adult. In other embodiments, the subject is a non-human vertebrate animal, including, without limitation, non-human primates, laboratory animals, livestock, racehorses, domesticated animals, and non-domesticated animals. In one embodiment, the term “human subjects” means a population of healthy human adults.

The singular forms “a”, “an” and “the” include plural reference unless the context clearly dictates otherwise.

As used herein the term “about” is used herein to mean approximately, roughly, around, or in the region of. When the term “about” is used in conjunction with a numerical range, it modifies that range by extending the boundaries above and below the numerical values set forth.

As used herein the term “variant” covers nucleotide or amino acid sequence variants which have about 99%, about 98%, about 97%, about 96%, about 95%, about 94%, about 93%, about 92%, about 91%, about 90%, about 85%, about 80%, about 75%, about 70%, or about 65% nucleotide identity, or about 99%, about 98%, about 97%, about 96%, about 95%, about 94%, about 93%, about 92%, about 91%, about 90%, about 85%, about 80%, about 75%, about 70%, or about 65% amino acid identity, including but not limited to variants comprising conservative, or non-conservative substitutions, deletions, insertions, duplications, or any other modification. The term variant as used herein includes functional and non-functional variants, and variants with reduced or altered activity.

As used herein, “PD-1” refers to programmed cell death protein. PD-1 is a protein on the surface of T and B cells that plays a role in regulating the immune system's response by down-regulating the immune system and promoting suppression of T cell inflammatory activity.

As used herein, “PAG” refers to glycosphingolipid-enriched microdomains 1. PAG is phosphorylated in T cells downstream of PD-1 signaling and contributes to the resulting functional inhibition of multiple cellular processes. PAG is a driver of murine tumor growth and immune evasion.

### T Cells and the Immune Synapse

T cells have surface receptors that can act as immune checkpoint receptors, such as PD-1. See Speiser D E, Ho P C, Verdeil G. Regulatory Circuits of T Cell Function in Cancer. Nat Rev Immunol. 2016 October; 16(10): pp. 599-611, incorporated by reference in its entirety herein. These receptors act as “checkpoints” to prevent excessive immune activation. However, cancer cells can exploit these checkpoint pathways to evade immune detection and attack. Sec Liu J, Chen Z, Li Y, Zhao W, Wu J and Zhang Z. PD-1/PD-L1 Checkpoint Inhibitors in Tumor Immunotherapy. Front. Pharmacol. 2021 September; pp. 12: p. 731798, incorporated by reference in its entirety herein. PD-1 is expressed on the surface of activated T cells while its ligands PD-L1 and PD-L2 are expressed on various cancer cells. See Liu, et al. (2021). The immune synapse is the interface between T cells and tumor cells. This interface, or microenvironment, includes the checkpoint receptors (e.g., PD-1, PDL-1, PDL-2) and other immune receptors and ligands needed for T cells to function. The immune synapse is organized into three compartments; every protein has a specific location. For example, with respect to proteins found on T cells, the T cell receptor (TCR) is localized to the center of the synapse, PD-1 and CD28 are located in the peripheral synapse, and LFA-1, CD43, and CD45 are found in the distal compartment of the synapse. See e.g., Delon J, Kaibuchi K, Germain R N. Exclusion of CD43 from the immunological synapse is mediated by phosphorylation-regulated relocation of the cytoskeletal adaptor moesin. Immunity. 2001 November; 15(5):691-701. Doi: 10.1016/s1074-7613(01)00231-x. PMID: 11728332. This organization is critical for the function of the synapse, where specific clusters of proteins are formed.

Cancer immunotherapy drugs block these T-cell checkpoint receptors, thereby enabling the immune system to recognize and destroy cancer cells more effectively. He X, Xu C. Immune Checkpoint Signaling and Cancer Immunotherapy. Cell Res. 2020 August; 30(8): pp. 660-669, incorporated by reference in its entirety herein. For example, PD-1 inhibitors prevent PD-1 on T cells from binding to its ligands PDL-1 and PDL-2 on tumor cells, thereby preventing the tumor cells from evading the T cell immune response. The FDA has approved seven monoclonal antibodies targeting PD-1 for cancer treatment. However, some individuals do not respond to this treatment.

PAG is phosphorylated after PD-1 is ligated by PD-L1 or PD-L2, such phosphorylation is associated with the inhibition of various T cell functions downstream of PD-1. See Nickolas T L, Schmidt-Ott K M, Canetta P, et al. Diagnostic and Prognostic Stratification in the Emergency Department Using Urinary Biomarkers of Nephron Damage: A Multicenter Prospective Cohort Study. Journal of the American College of Cardiology. 2012; 59(3):246-255. PAG localizes to the immune synapse between a T cell and APC. This localization is essential for its function in the PD-1 pathway. See Nickolas T L, Schmidt-Ott K M, Canetta P, et al. Diagnostic and Prognostic Stratification in the Emergency Department Using Urinary Biomarkers of Nephron Damage: A Multicenter Prospective Cohort Study.

PAG is phosphorylated in T cells downstream of PD-1 signaling and contributes to the resulting functional inhibition of multiple cellular processes. Furthermore, PAG expression is negatively correlated with survival in multiple human tumors and is a driver of murine tumor growth and immune evasion. Described herein are antibodies that targets the extracellular domain of human PAG, with cross-reactivity to murine PAG. These antibodies bind to extracellular PAG on intact cells and affects T cell activation. Administration of anti-PAG monoclonal antibody in combination with anti-PD-1 antibody to mice bearing MC38 tumors limited tumor growth and enhanced T cell infiltration to tumors.

Without intending to be bound by any particular theory, the design of antibodies for the prevention or treatment of cancer is improved by taking into account the co-localization of PAG with PD-1 in the context of the immune synapse. Without intending to be bound by any particular theory, in addition to blocking phosphorylation of PAG, t changing the co-location of PAG with PD-1 within the different compartments of the immune synapse could serve as an alternative, efficient approach to treating cancer patients.

### Anti-PAG Antibodies

Disclosed herein are novel monoclonal antibodies that, in some embodiments, are useful for preventing or treating disease (e.g., cancer). One approach to cancer treatment consists of using monoclonal antibodies to target PD-1 binding on T cells. However, a significant number of patients do not respond to this treatment. Described herein is the generation of anti-PAG antibodies that can bind to the extracellular portion of PAG. The antibodies removed the PAG protein from the center of the immune synapse. In comparison to the administration of anti-PD-1 antibodies alone, this technology shows superior induction of cytokine secretion and primary T cell killing of tumor cells in vitro. As such, this approach offers a more efficacious alternative to current immunotherapies for treating cancer patients.

Unless otherwise indicated, the practice of aspects of the present invention can employ conventional techniques of cell biology, cell culture, molecular biology, transgenic biology, microbiology, recombinant DNA, and immunology, which are within the skill of the art. Such techniques are explained fully in the literature. See, e.g., Molecular Cloning A Laboratory Manual, 3rd Ed., ed. by Sambrook (2001), Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription and Translation (B. D. Hames & S. J. Higgins eds. 1984); Culture Of Animal Cells (R. I. Freshney, Alan R. Liss, Inc., 1987); Immobilized Cells and Enzymes (IRL Press, 1986); B. Perbal, A Practical Guide To Molecular Cloning (1984); the series, Methods In Enzymology (Academic Press, Inc., N.Y.), specifically, Methods In Enzymology, Vols. 154 and 155 (Wu et al. eds.); Gene Transfer Vectors For Mammalian Cells (J. H. Miller and M. P. Calos eds., 1987, Cold Spring Harbor Laboratory); Immunochemical Methods In Cell And Molecular Biology (Caner and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986) and subsequent versions thereof. All patents, patent applications and references cited herein are incorporated by reference in their entireties.

One skilled in the art can obtain a protein in several ways, including, but not limited to isolating the protein via biochemical means or expressing a nucleotide sequence encoding the protein of interest by genetic engineering methods.

A protein is encoded by a nucleic acid (including, for example, genomic DNA, complementary DNA (cDNA), synthetic DNA, as well as any form of corresponding RNA). For example, it can be encoded by a recombinant nucleic acid of a gene. The proteins of the invention can be obtained from various sources and can be produced according to various techniques known in the art. For example, a nucleic acid that encodes a protein can be obtained by screening DNA libraries, or by amplification from a natural source. A protein can be a fragment or portion thereof. The nucleic acids encoding a protein can be produced via recombinant DNA technology and such recombinant nucleic acids can be prepared by conventional techniques, including chemical synthesis, genetic engineering, enzymatic techniques, or a combination thereof.

Protein variants can include amino acid sequence modifications. For example, amino acid sequence modifications fall into one or more of three classes: substitutional, insertional or deletional variants. Insertions can include amino and/or carboxyl terminal fusions as well as intrasequence insertions of single or multiple amino acid residues. Ordinarily, insertions will be smaller than those of amino or carboxyl terminal fusions, for example, on the order of one to four residues. Deletions are characterized by the removal of one or more amino acid residues from the protein sequence. These variants ordinarily are prepared by site-specific mutagenesis of nucleotides in the DNA encoding the protein, thereby producing DNA encoding the variant, and thereafter expressing the DNA in recombinant cell culture or using cell-free methods known in the art.

Nucleic acid sequences encoding a molecule can be synthesized, in whole or in part, using chemical methods known in the art. Alternatively, a protein can be produced using chemical methods to synthesize its amino acid sequence, such as by direct peptide synthesis using solid-phase techniques. Protein synthesis can either be performed using manual techniques or by automation. For example, automated synthesis can be achieved using Applied Biosystems 431A Peptide Synthesizer (Perkin Elmer).

Optionally, polypeptide fragments can be separately synthesized and combined using chemical methods to produce a full-length molecule. For example, these methods can synthesize a protein of the invention.

An anti-PAG antibody can be obtained by purification from a sample. Non-limiting purification methods include size exclusion chromatography, ammonium sulfate fractionation, ion exchange chromatography, affinity chromatography, and preparative gel electrophoresis.

Polypeptide modifications are well known to those of skill and have been described in detail in the scientific literature. Several common modifications, such as glycosylation, lipid attachment, sulfation, gamma-carboxylation of glutamic acid residues, hydroxylation and ADP-ribosylation, for instance, are described in most basic texts, such as, for instance Creighton, Protein Structure and Molecular Properties, 2^{nd} ed., W. H. Freeman and Company (1993). Many detailed reviews are available on this subject, such as, for example, those provided by Wold, in Johnson (ed.), Posttranslational Covalent Modification of Proteins, pgs. 1-12, Academic Press (1983); Seifter et al., Meth. Enzymol. 182:626-646 (1990) and Rattan et al., Ann. N.Y. Acad. Sci. 663:48-62 (1992).

In some embodiments, described herein are polypeptides or proteins that have been post-translationally modified. In one embodiment, polypeptides or proteins can be modified enzymatically or chemically, by addition or removal of a post-translational modification. For example, a polypeptide or protein can be glycosylated or deglycosylated enzymatically. Similarly, polypeptides can be phosphorylated using a purified kinase, such as a MAP kinase (e.g, p38, ERK, or JNK) or a tyrosine kinase (e.g., Src or erbB2). A polypeptide or protein can also be modified through synthetic chemistry. Alternatively, one can isolate the polypeptide or protein of interest from a cell or tissue that expresses the polypeptide with the desired post-translational modification. In another embodiment, a nucleic acid molecule encoding the polypeptide or protein of interest is introduced into a host cell that is capable of post-translationally modifying the encoded polypeptide(s) in the desired fashion. If the polypeptide or protein does not contain a motif for a desired post-translational modification, one can alter the post-translational modification by mutating the nucleic acid sequence of a nucleic acid molecule encoding the polypeptide(s) so that it contains a site for the desired post-translational modification. The nucleic acid molecule can also be introduced into a host cell that is capable of post-translationally modifying the encoded polypeptide(s). Similarly, one can delete sites that are post-translationally modified by mutating the nucleic acid sequence so that the encoded polypeptide(s) does not contain the post-translational modification motif, or by introducing the native nucleic acid molecule into a host cell that is not capable of post-translationally modifying the encoded polypeptide(s).

Useful post-synthetic (and post-translational) modifications include conjugation to detectable labels, such as fluorophores. A wide variety of amine-reactive and thiol-reactive fluorophore derivatives have been synthesized that react under nondenaturing conditions with N-terminal amino groups and epsilon amino groups of lysine residues, on the one hand, and with free thiol groups of cysteine residues, on the other. Other labels that usefully can be conjugated to polypeptides or proteins disclosed herein include radioactive labels, echosonographic contrast reagents, and MRI contrast agents. Kits are available commercially that permit conjugation of proteins to a variety of amine-reactive or thiol-reactive fluorophores: e.g., kits for conjugating proteins to Alexa Fluor 350, Alexa Fluor 430, Fluorescein-EX, Alexa Fluor 488, Oregon Green 488, Alexa Fluor 532, Alexa Fluor 546, Alexa Fluor 546, Alexa Fluor 568, Alexa Fluor 594, Texas Red-X, BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, and Texas Red.

There are five classes of human antibodies (i.e., IgA, IgD, IgE, IgG, and IgM) and each have various isotypes (e.g., IgG1, IgG2, IgG3, IgG4, IgA1, and IgA2). In some embodiments, the antibodies disclosed herein belong to the IgG class. IgG can be divided into four subclasses: IgG1, IgG2, IgG3, and IgG4. Each subclass has a unique profile with respect to antigen binding, immune complex formation, complement activation, triggering of effector cells, half-life, and placental transport. E.g., see Gestur Vidarsson, et al., IgG Subclasses and Allotypes: From Structure to Effector Functions, 5 Frontiers in Immunology 520 (2014), incorporated by reference herein in its entirety. The term “immunoglobulin” (Ig) is used interchangeably with “antibody” herein.

The IgG immunoglobulin molecule consists of four polypeptide chains, two identical light (L) chains and two identical heavy (H) chains. Disulfide bonds join the four chains in a “Y” configuration wherein the light chains bracket the heavy chains starting at the mouth of the “Y” and continuing through the variable region to the dual ends of the “Y”. Each L chain is linked to an H chain by one covalent disulfide bond, while the two H chains are linked to each other by one or more disulfide bonds depending on the H chain isotype. Each H and L chain also has regularly spaced intrachain disulfide bridges. Each heavy chain consists of an N-terminal variable domain (VH) and three constant domains (CH1, CH2, CH3), with an additional “hinge region” between CH1 and CH2. Similarly, the light chains consist of an N-terminal variable domain (VL) and a constant domain (CL). The variable domains of the heavy chain and light chain may be referred to as “VH” and “VL”, respectively. These domains are generally the most variable parts of the antibody (relative to other antibodies of the same class) and contain the antigen binding sites. The VL is aligned with the VH and the CL is aligned with the first constant domain of the heavy chain (CH1). The pairing of a VH and VL together forms a single antigen-binding site. The part of the antibody formed by the lower hinge region and the CH2/CH3 domains of the heavy chain is called “Fc” (“fragment crystalline”). See e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6, incorporated by reference herein in its entirety.

The variability in an antibody sequence is concentrated in three segments called complementarity determining regions (CDRs) (also called hypervariable regions (HVRs)) both in the light-chain and the heavy-chain variable domains. The more highly conserved portions of variable domains are called the framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies. See Kabat et al, Sequences of Immunological Interest, Fifth Edition, National Institute of Health, Bethesda, MD (1991), incorporated by reference in its entirety herein. The constant domains are not involved directly in the binding of antibodies to an antigen but exhibit various effector functions, such as the participation of the antibody in antibody-dependent cellular toxicity.

By way of example, CDRs may be defined using the nomenclature described by Kabat et al. (1991, NIH Publication 91-3242, National Technical Information Service, Springfield, Va.), incorporated by reference in its entirety herein. Specifically, residues 31-35 (CDR-H1), 50-65 (CDR-H2), and 95-102 (CDR-H3) in the heavy chain variable region and residues 24-34 (CDR-L1), 50-56 (CDR-L2), and 89-97 (CDR-L3) in the light chain variable region.

The antibodies disclosed herein (e.g., monoclonal antibodies) includes, but is not limited to, full-length antibodies and also includes other immunologically reactive/antigen-binding molecules. The antibodies of the various embodiments disclosed herein can include one or more of synthetic antibodies, monoclonal antibodies, oligoclonal or polyclonal antibodies, multiclonal antibodies, recombinantly produced antibodies, intrabodies, monospecific antibodies, monovalent antibodies, multispecific antibodies, multivalent antibodies, bispecific antibodies, bivalent antibodies, tetravalent antibodies, human antibodies, humanized antibodies, chimeric antibodies, CDR-grafted antibodies, primatized antibodies, Fab fragments, F(ab′) fragments, F(ab′)₂ fragments, Fv fragments, single-chain FvFcs (scFv-Fc), single-chain Fvs (scFv), Dabs, nanobodies, anti-idiotypic (anti-Id) antibodies, and any other immunologically-reactive/antigen-binding molecules. In some embodiments, the antibody is a monoclonal antibody.

In some embodiments, the monoclonal antibody comprises a first, second, third and fourth chain. In some embodiments, the first and third chains each comprise a VH domain and the second and fourth chains each comprise a VL domain. In some embodiments, the first and third chains each further comprise a CH1 domain, a hinge domain, and a Fc domain. In some embodiments, the second and fourth chains each further comprise a CL domain. The pairing of the VH and VL of the first and second chains together forms a single antigen-binding site specific for an epitope on PAG and the pairing of the VH and VL of the third and fourth chains together forms a single antigen-binding site specific for the same epitope. In some embodiments, the first and second chains are linked by one or more covalent disulfide bonds and the third and fourth chains are linked by one or more covalent disulfide bonds. In some embodiments, the first and third chains are linked by one or more disulfide bonds.

However, the antibodies disclosed herein are not limited to full-length IgG like antibodies. Other immunologically reactive/antigen-binding molecules are also contemplated herein and a person of skill in the art can readily synthesize such molecules using the sequences and identified domains of the heavy and light chains of the anti-PAG antibodies disclosed here. For example, in some embodiments, the monoclonal antibody comprises a first and second chain that associate together. In some embodiments, the first chain and second chain each comprises an scFv with specificity for an epitope on PAG. An scFv comprises a variable heavy domain and variable light chain domain separated by a linker. In some embodiments, the linker is a glycine-serine linker. In some embodiments, the Fc domain of the first chain comprises knob mutations and the Fc domain of the second chain comprise hole mutations, or vice versa.

For example, in some embodiments, the monoclonal antibody comprises a first and second chain that associate together. In some embodiments, the first chain and second chain each comprise two scFvs with specificity for an epitope on PAG and the first and second chains each further comprise a Fc domain. An scFv comprises a variable heavy domain and variable light chain domain separated by a linker. In some embodiments, the linker is a glycine-serine linker. In some embodiments, the Fc domain of the first chain comprises knob mutations and the Fc domain of the second chain comprises hole mutations, or vice versa. In some embodiments, the antibody is a scFv-Fc-scFv antibody comprising a first and second chain that associate together, each chain comprising a first variable heavy chain (VH) domain, a first linker, a first variable light chain (VL) domain, an Fc domain, a second variable heavy chain (VH) domain, a second linker, and a second variable light chain (VL) domain.

In some embodiments, the monoclonal antibodies disclosed herein contain various modifications, substitutions, additions, or deletions to the variable or binding regions of one or more arms of an anti-PAG antibody disclosed herein. In some embodiments, the monoclonal antibodies disclosed herein may contain substitutions or modifications of the constant region (i.e., the Fc domain). The antibodies disclosed herein may contain one or more additional amino acid residue substitutions, mutations and/or modifications, which result in a compound with preferred characteristics including, but not limited to: altered pharmacokinetics, increased serum half-life, increased binding affinity, reduced binding affinity, reduced immunogenicity, increased production, altered Fc ligand binding, enhanced or reduced ADCC or CDC activity, altered glycosylation and/or disulfide bonds and modified binding specificity.

### Amino Acid Sequences of Anti-PAG Antibodies

In some embodiments, the anti-PAG antibody comprises two polypeptide heavy chains each comprising a variable heavy chain (VH) domain, CH1 domain, a hinge domain, a Fc domain (CH2-CH3), and two polypeptide light chains comprising a variable light chain (VL) domain and a CL domain.

In some embodiments, the amino acid sequence of the anti-PAG antibody VH domain comprises SEQ ID NO: 5. Bolded amino acids represent the CDR-1 (GYTFTSYV (SEQ ID NO:6)), CDR-2 (TYPYNDGT (SEQ ID NO:7)), and CDR-3 (ARYKYGQGFAY (SEQ ID NO:8)), respectively. EVQLQQSGPELVKPGASVKMSCKASGYTFTSYVMHWVKQKPGQGLEWIGYIYPYN DGTKYNEKFKGKATLTSDKSSSTAYMELSSLTSEDSAVYYCARYKYGQGFAYWGQ GTRVTVSA (SEQ ID NO: 5). In some embodiments, the amino acid sequence of the anti-PAG antibody heavy chain comprises a signal peptide (for example, but not limited to, MGWSCIILFLVATATGVHS (SEQ ID NO:9)) immediately followed by SEQ ID NO: 5. In some embodiments, the signal peptide is cleaved during post-translational modifications that occur in vitro or in vivo. In some embodiments, a nucleic acid sequence encoding the amino acid sequence of the anti-PAG antibody heavy chain comprises a nucleic acid sequence encoding a signal peptide (for example, but not limited to, SEQ ID NO:9) immediately followed by a nucleic acid sequence encoding SEQ ID NO: 5.

In some embodiments, the anti-PAG antibody comprises a polypeptide heavy chain comprising a variable heavy chain (VH) domain comprising SEQ ID NO: 5 followed by a constant region comprising a CH1 domain, a hinge domain, a Fc domain (CH2-CH3). In some embodiments the constant region comprises a CH1 domain, a hinge domain, and a Fc domain (CH2-CH3) of a mouse IgG1, IgG2, IgG3, or IgG4 constant region. In some embodiments the constant region comprises a CH1 domain, a hinge domain, and a Fc domain (CH2-CH3) of a mouse IgG1 constant region. In some embodiments the constant region comprises a CH1 domain, a hinge domain, and a Fc domain (CH2-CH3) of a human IgG1, IgG2, IgG3, or IgG4 constant region. In some embodiments the constant region comprises a CH1 domain, a hinge domain, and a Fc domain (CH2-CH3) of a human IgG1 constant region.

In some embodiments, the amino acid sequence of the anti-PAG antibody heavy chain comprises SEQ ID NO: 10. Underlined amino acids represent the VH domain. Underlined and bolded amino acids represent CDR-1 (SEQ ID NO:6), CDR-2 (SEQ ID NO: 7), and CDR-3 (SEQ ID NO:8), respectively. Italicized and bold amino acids represent the CH1 domain. Italicized and underlined amino acids represent the hinge domain. Italicized amino acids represent the Fc domain. Double-underlined amino acids represent the additional Fc domain. The italicized amino acids can be replaced with any other constant domain known in the art.

(SEQ ID NO: 10)
EVQLQQSGPELVKPGASVKMSCKASGYTFTSYVMHWVKQKPGQGLEWIGY
IYPYNDGTKYNEKFKGKATLTSDKSSSTAYMELSSLTSEDSAVYYCARYK
YGQGFAYWGQGTRVTVSAAKTTPPSVYPLAPGSAAQTNSMVTLGCLVKGY
FPEPVTVTWNSGSLSSGVHTFPAVLQSDLYTLSSSVTVPSSTWPSETVTC
NVAHPASSTKVDKKIVPRDCGCKPCICTVPEVSSVFIFPPKPKDVLTITL
TPKVTCVVVDISKDDPEVQFSWFVDDVEVHTAQTQPREEQFNSTFRSVSE
LPIMHQDWLNGKEFKCRVNSAAFPAPIEKTISKTKGRPKAPQVYTIPPPK
EQMAKDKVSLTCMITDFFPEDITVEWQWNGQPAENYKNTQPIMDTDGSYF
VYSKLNVQKSNWEAGNTFTCSVLHEGLHNHHTEKSLSHSPGK.

In some embodiments, the amino acid sequence of the anti-PAG antibody heavy chain comprises SEQ ID NO: 10 without the terminal lysine residue. In some embodiments, the amino acid sequence of the anti-PAG antibody heavy chain comprises a signal peptide (for example, but not limited to (SEQ ID NO:9)) immediately followed by SEQ ID NO: 10 or SEQ ID NO: 10 without the terminal lysine residue. In some embodiments, the signal peptide is cleaved during post-translational modifications that occur in vitro or in vivo. In some embodiments, a nucleic acid sequence encoding the amino acid sequence of the anti-PAG antibody heavy chain comprises a nucleic acid sequence encoding a signal peptide (for example, but not limited to, SEQ ID NO:9) immediately followed by a nucleic acid sequence encoding SEQ ID NO: 10 or SEQ ID NO: 10 without the terminal lysine residue.

In some embodiments, the amino acid sequence of the anti-PAG antibody VL domain comprises SEQ ID NO: 11. Bolded amino acids represent the CDR-1 (ENIYSN (SEQ ID NO:12)), CDR-2 (AAT (SEQ ID NO:13), and CDR-3 (QHFWGTPWT (SEQ ID NO: 14), respectively. DIQMTQSPASLSVSVGETVTITCRASENIYSNLAWYQQKQGKSPQLLVYAATNLAD GVPSRFSGSGSGTQYSLKINSLQSEDFGSYYCQHFWGTPWTFGGGTKLEIK (SEQ ID NO: 11). In some embodiments, the amino acid sequence of the anti-PAG antibody light chain comprises a signal peptide (for example, but not limited to, SEQ ID NO:9) immediately followed by SEQ ID NO: 11. In some embodiments, the signal peptide is cleaved during post-translational modifications that occur in vitro or in vivo. In some embodiments, a nucleic acid sequence encoding the amino acid sequence of the anti-PAG antibody light chain comprises a nucleic acid sequence encoding a signal peptide (for example, but not limited to, SEQ ID NO:9) immediately followed by a nucleic acid sequence encoding SEQ ID NO: 11.

In some embodiments, the anti-PAG antibody comprises a polypeptide light chain comprising a variable light chain (VL) domain comprising SEQ ID NO: 11 followed by a constant region comprising a CL domain. In some embodiments the constant region comprises a CL domain of a mouse Ig kappa chain or Ig lambda chain. In some embodiments the constant region comprises a CL of a mouse Ig kappa chain. In some embodiments the constant region comprises a CL domain of a human Ig kappa chain or Ig lambda chain. In some embodiments the constant region comprises a CL of a human Ig kappa chain.

In some embodiments, the amino acid sequence of the anti-PAG antibody light chain comprises SEQ ID NO: 15. Underlined amino acids represent the VL domain. Underlined and bolded amino acids represent CDR-1 (SEQ ID NO:12), CDR-2 (SEQ ID NO: 13), and CDR-3 (SEQ ID NO:14), respectively. Italicized amino acids represent the CL domain. The italicized amino acids can be replaced with any another constant domain known in the art.

(SEQ ID NO: 15)
DIQMTQSPASLSVSVGETVTITCRASENIYSNLAWYQQKQGKSPQLLVYA
ATNLADGVPSRFSGSGSGTQYSLKINSLQSEDFGSYYCQHFWGTPWTFGG
GTKLEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKT
STSPIVKSFNRNEC.

In some embodiments, the amino acid sequence of the anti-PAG antibody heavy chain comprises a signal peptide (for example, but not limited to (SEQ ID NO:9)) immediately followed by SEQ ID NO: 15. In some embodiments, the signal peptide is cleaved during post-translational modifications that occur in vitro or in vivo. In some embodiments, a nucleic acid sequence encoding the amino acid sequence of the anti-PAG antibody light chain comprises a nucleic acid sequence encoding a signal peptide (for example, but not limited to, SEQ ID NO:9) immediately followed by a nucleic acid sequence encoding SEQ ID NO: 15.

In some embodiments, the monoclonal antibody comprises a first and third chain each comprising SEQ ID NO: 5 and a second and fourth chains each comprising SEQ ID NO: 11. In some embodiments, the monoclonal antibody comprises a first and third chain each comprising SEQ ID NO: 10 and a second and fourth chains each comprising SEQ ID NO: 15. In some embodiments, the monoclonal antibody comprises a first and third chain each comprising SEQ ID NO: 5 followed by a constant region comprising a CH1 domain, a hinge domain, a Fc domain (CH2-CH3) and a second and fourth chains each comprising SEQ ID NO: 11 followed by a constant region comprising a CL domain, wherein the constant region of the first and third chain comprises a CH1 domain, a hinge domain, and a Fc domain (CH2-CH3) of a mouse or human IgG1, IgG2, IgG3, or IgG4 constant region and the constant region of the second and fourth chain comprises a CL domain of a mouse or human Ig kappa chain or Ig lambda chain. In some embodiments, the first and second chains are linked by one or more covalent disulfide bonds and the third and fourth chains are linked by one or more covalent disulfide bonds. In some embodiments, the first and third chains are linked by one or more disulfide bonds.

In some embodiments, the anti-PAG antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 5. In some embodiments, the anti-PAG antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 11. In some embodiments, the anti-PAG antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 10. In some embodiments, the anti-PAG antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 15. In some embodiments, the anti-PAG antibody comprises an amino acid sequence comprising the CDRs of the variable heavy chain domain and the CDRs of the variable light chain domain of the anti-PAG antibody, wherein the CDR sequences are SEQ ID NO: 6 GYTFTSYV (CDR-H1), SEQ ID NO: 7 IYPYNDGT (CDR-H2), SEQ ID NO: 8 ARYKYGQGFAY (CDR-H3), SEQ ID NO: 12 ENIYSN (CDR-L1), SEQ ID NO: 13 AAT (CDR-L2), and SEQ ID NO: 14 QHFWGTPWT (CDR-L3). In some embodiments, the framework regions (FRs) of the variable heavy chain domain and the FRs of the variable light chain domain of the anti-PAG antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 5 and SEQ ID NO: 11, respectively.

In some embodiments, one or more amino acids of an amino acid sequence encoding one or more CDRs of SEQ ID NO: 5 or SEQ ID NO: 11 is substituted. In some embodiments, one or more amino acids of an amino acid sequence encoding one or more variable heavy chain CDRs of SEQ ID NO: 5 is substituted. In some embodiments, one or more amino acids of an amino acid sequence encoding one or more variable light chain CDRs of SEQ ID NO: 11 is substituted. In some embodiments, one or more amino acids of an amino acid sequence encoding one or more CDRs of SEQ ID NO: 10 or SEQ ID NO: 15 is substituted. In some embodiments, one or more amino acids of an amino acid sequence encoding one or more variable heavy chain CDRs of SEQ ID NO: 10 is substituted. In some embodiments, one or more amino acids of an amino acid sequence encoding one or more variable light chain CDRs of SEQ ID NO: 15 is substituted.

In some embodiments the monoclonal antibody is a scFv antibody comprising polypeptide chain comprising a variable heavy chain (VH) domain of SEQ ID NO: 5, a linker (e.g., but not limited to, GGGGSGGGGSGGGGS (SEQ ID NO:26)), and a variable light chain (VL) domain of SEQ ID NO: 11.

In some embodiments the monoclonal antibody is a scFv-Fc antibody comprising a first and second chain that associate together, each chain comprising a variable heavy chain (VH) domain of SEQ ID NO: 5, a linker (e.g., but not limited to SEQ ID NO: 26), a variable light chain (VL) domain of SEQ ID NO: 11, and an Fc domain. In some embodiments, the Fc domain comprises the Fc domain of SEQ ID NO: 10. In some embodiments, the Fc domain comprises the Fc domain of a mouse IgG1, IgG2, IgG3, or IgG4 constant region. In some embodiments, the Fc domain comprises the Fc domain of a mouse IgG1 constant region. In some embodiments, the Fc domain comprises the Fc domain of a human IgG1, IgG2, IgG3, or IgG4 constant region. In some embodiments, the Fc domain comprises the Fc domain of a human IgG1 constant region.

In some embodiments the monoclonal antibody is a scFv-Fc-scFv antibody comprising a first and second chain that associate together, each chain comprising a first variable heavy chain (VH) domain of SEQ ID NO: 5, a first linker (e.g., but not limited to SEQ ID NO: 26), a first variable light chain (VL) domain of SEQ ID NO: 11, an Fc domain, a second variable heavy chain (VH) domain of SEQ ID NO: 5, a second linker (e.g., but not limited to SEQ ID NO: 26), and a second variable light chain (VL) domain of SEQ ID NO: 11. In some embodiments, the Fc domain comprises the Fc domain of SEQ ID NO: 10. In some embodiments, the Fc domain comprises the Fc domain of a mouse IgG1, IgG2, IgG3, or IgG4 constant region. In some embodiments, the Fc domain comprises the Fc domain of a mouse IgG1 constant region. In some embodiments, the Fc domain comprises the Fc domain of a human IgG1, IgG2, IgG3, or IgG4 constant region. In some embodiments, the Fc domain comprises the Fc domain of a human IgG1 constant region.

The antibody comprising a dimer of the heterodimer comprising SEQ ID NO: 10 and SEQ ID NO: 15 is represented by antibody “7M16A” in embodiments described and depicted in this disclosure.

In some embodiments, the amino acid sequence of the anti-PAG antibody VH domain comprises SEQ ID NO: 16. Bolded amino acids represent the CDR-1 (GYIFTNYG (SEQ ID NO:17)), CDR-2 (INPYTGEA (SEQ ID NO:18)), and CDR-3 (AKTGTTY (SEQ ID NO: 19)), respectively. QIQLVQSGPELKKPGETVKISCKASGYIFTNYGMNWVKQAPGKGLKWMGWINPYT GEATYDDDFKGRFAFSLETSANTAYLQINNLKNEDTATYFCAKTGTTYWGQGTLV TVSA (SEQ ID NO: 16). In some embodiments, the amino acid sequence of the anti-PAG antibody heavy chain comprises a signal peptide (for example, but not limited to, SEQ ID NO: 9) immediately followed by SEQ ID NO: 16. In some embodiments, the signal peptide is cleaved during post-translational modifications that occur in vitro or in vivo. In some embodiments, a nucleic acid sequence encoding the amino acid sequence of the anti-PAG antibody heavy chain comprises a nucleic acid sequence encoding a signal peptide (for example, but not limited to, SEQ ID NO:9) immediately followed by a nucleic acid sequence encoding SEQ ID NO: 16.

In some embodiments, the anti-PAG antibody comprises a polypeptide heavy chain comprising a variable heavy chain (VH) domain comprising SEQ ID NO: 16 followed by a constant region comprising a CH1 domain, a hinge domain, a Fc domain (CH2-CH3). In some embodiments the constant region comprises a CH1 domain, a hinge domain, and a Fc domain (CH2-CH3) of a mouse IgG1, IgG2, IgG3, or IgG4 constant region. In some embodiments the constant region comprises a CH1 domain, a hinge domain, and a Fc domain (CH2-CH3) of a mouse IgG1 constant region. In some embodiments the constant region comprises a CH1 domain, a hinge domain, and a Fc domain (CH2-CH3) of a human IgG1, IgG2, IgG3, or IgG4 constant region. In some embodiments the constant region comprises a CH1 domain, a hinge domain, and a Fc domain (CH2-CH3) of a human IgG1 constant region.

In some embodiments, the amino acid sequence of the anti-PAG antibody heavy chain comprises SEQ ID NO: 20. Underlined amino acids represent the VH domain. Underlined and bolded amino acids represent CDR-1 (SEQ ID NO:17), CDR-2 (SEQ ID NO: 18), and CDR-3 (SEQ ID NO:19), respectively. Italicized and bold amino acids represent the CH1 domain. Italicized and underlined amino acids represent the hinge domain. Italicized amino acids represent the Fc domain. Double underlined amino acids represent the additional Fc domain. The italicized amino acids can be replaced with any another constant domain known in the art.

(SEQ ID NO: 20)
QIQLVQSGPELKKPGETVKISCKASGYIFTNYGMNWVKQAPGKGLKWMGW
INPYTGEATYDDDFKGRFAFSLETSANTAYLQINNLKNEDTATYFCAKTG
TTYWGQGTLVTVSAAKTTPPSVYPLAPGSAAQTNSMVTLGCLVKGYFPEP
VTVTWNSGSLSSGVHTFPAVLQSDLYTLSSSVTVPSSTWPSETVTCNVAH
PASSTKVDKKIVPRDCGCKPCICTVPEVSSVFIFPPKPKDVLTITLTPKV
TCVVVDISKDDPEVQFSWFVDDVEVHTAQTQPREEQFNSTFRSVSELPIM
HQDWLNGKEFKCRVNSAAFPAPIEKTISKTKGRPKAPQVYTIPPPKEQMA
KDKVSLTCMITDFFPEDITVEWQWNGQPAENYKNTQPIMDTDGSYFVYSK
LNVQKSNWEAGNTFTCSVLHEGLHNHHTEKSLSHSPGK.

In some embodiments, the amino acid sequence of the anti-PAG antibody heavy chain comprises SEQ ID NO: 20 without the terminal lysine residue. In some embodiments, the amino acid sequence of the anti-PAG antibody heavy chain comprises a signal peptide (for example, but not limited to (SEQ ID NO:9)) immediately followed by SEQ ID NO: 20 or SEQ ID NO: 20 without the terminal lysine residue. In some embodiments, the signal peptide is cleaved during post-translational modifications that occur in vitro or in vivo. In some embodiments, a nucleic acid sequence encoding the amino acid sequence of the anti-PAG antibody heavy chain comprises a nucleic acid sequence encoding a signal peptide (for example, but not limited to, SEQ ID NO:9) immediately followed by a nucleic acid sequence encoding SEQ ID NO: 20 or SEQ ID NO: 20 without the terminal lysine residue.

In some embodiments, the amino acid sequence of the anti-PAG antibody VL domain comprises SEQ ID NO: 21. Bolded amino acids represent the CDR-1 (SSVSY (SEQ ID NO: 22)), CDR-2 (DTS (SEQ ID NO:23), and CDR-3 (QQWSSNPLT (SEQ ID NO:24), respectively. QIVLTQSPAIMSASPGEKVTMTCSASSSVSYMYWYQQKSGTSPKRWIYDTSKLASGV PARESGSGSGTSYSLTINNMEAEDAATYYCQQWSSNPLTFGAGTKLELK (SEQ ID NO: 21). In some embodiments, the amino acid sequence of the anti-PAG antibody light chain comprises a signal peptide (for example, but not limited to, SEQ ID NO:9) immediately followed by SEQ ID NO: 21. In some embodiments, the signal peptide is cleaved during post-translational modifications that occur in vitro or in vivo. In some embodiments, a nucleic acid sequence encoding the amino acid sequence of the anti-PAG antibody light chain comprises a nucleic acid sequence encoding a signal peptide (for example, but not limited to, SEQ ID NO:9) immediately followed by a nucleic acid sequence encoding SEQ ID NO: 21.

In some embodiments, the anti-PAG antibody comprises a polypeptide light chain comprising a variable light chain (VL) domain comprising SEQ ID NO: 21 followed by a constant region comprising a CL domain. In some embodiments the constant region comprises a CL domain of a mouse Ig kappa chain or Ig lambda chain. In some embodiments the constant region comprises a CL of a mouse Ig kappa chain. In some embodiments the constant region comprises a CL domain of a human Ig kappa chain or Ig lambda chain. In some embodiments the constant region comprises a CL of a huma Ig kappa chain.

In some embodiments, the amino acid sequence of the anti-PAG antibody light chain comprises SEQ ID NO: 25. Underlined amino acids represent the VL domain. Underlined and bolded amino acids represent CDR-1 (SEQ ID NO:22), CDR-2 (SEQ ID NO: 23), and CDR-3 (SEQ ID NO:24), respectively. Italicized amino acids represent the CL domain. The italicized amino acids can be replaced with any another constant domain known in the art.

(SEQ ID NO: 25)
QIVLTQSPAIMSASPGEKVTMTCSASSSVSYMYWYQQKSGTSPKRWIYDT
SKLASGVPARFSGSGSGTSYSLTINNMEAEDAATYYCQQWSSNPLTFGAG
TKLELKRADAAPTVSIFPPSSEQLTSGGASVVCFLNNFYPKDINVKWKID
GSERQNGVLNSWTDQDSKDSTYSMSSTLTLTKDEYERHNSYTCEATHKTS
TSPIVKSFNRNEC.

In some embodiments, the amino acid sequence of the anti-PAG antibody heavy chain comprises a signal peptide (for example, but not limited to (SEQ ID NO:9)) immediately followed by SEQ ID NO: 25. In some embodiments, the signal peptide is cleaved during post-translational modifications that occur in vitro or in vivo. In some embodiments, a nucleic acid sequence encoding the amino acid sequence of the anti-PAG antibody light chain comprises a nucleic acid sequence encoding a signal peptide (for example, but not limited to, SEQ ID NO:9) immediately followed by a nucleic acid sequence encoding SEQ ID NO: 25. In some embodiments, the monoclonal antibody comprises a first and third chain each comprising SEQ ID NO: 16 and a second and fourth chains each comprising SEQ ID NO: 21. In some embodiments, the monoclonal antibody comprises a first and third chain each comprising SEQ ID NO: 20 and a second and fourth chains each comprising SEQ ID NO: 25. In some embodiments, the monoclonal antibody comprises a first and third chain each comprising SEQ ID NO: 16 followed by a constant region comprising a CH1 domain, a hinge domain, a Fc domain (CH2-CH3) and a second and fourth chains each comprising SEQ ID NO: 21 followed by a constant region comprising a CL domain, wherein the constant region of the first and third chain comprises a CH1 domain, a hinge domain, and a Fc domain (CH2-CH3) of a mouse or human IgG1, IgG2, IgG3, or IgG4 constant region and the constant region of the second and fourth chain comprises a CL domain of a mouse or human Ig kappa chain or Ig lambda chain. In some embodiments, the first and second chains are linked by one or more covalent disulfide bonds and the third and fourth chains are linked by one or more covalent disulfide bonds. In some embodiments, the first and third chains are linked by one or more disulfide bonds.

In some embodiments, the anti-PAG antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 16. In some embodiments, the anti-PAG antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 21. In some embodiments, the anti-PAG antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 20. In some embodiments, the anti-PAG antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 25. In some embodiments, the anti-PAG antibody comprises an amino acid sequence comprising the CDRs of the variable heavy chain domain and the CDRs of the variable light chain domain of the anti-PAG antibody, wherein the CDR sequences are SEQ ID NO: 17 (CDR-H1), SEQ ID NO: 18 (CDR-H2), SEQ ID NO: 19 (CDR-H3), SEQ ID NO: 22 (CDR-L1), SEQ ID NO: 23 (CDR-L2), and SEQ ID NO: 24 (CDR-L3). In some embodiments, the framework regions (FRs) of the variable heavy chain domain and the FRs of the variable light chain domain of the anti-PAG antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 16 and SEQ ID NO: 21, respectively.

In some embodiments, one or more amino acids of an amino acid sequence encoding one or more CDRs of SEQ ID NO: 16 or SEQ ID NO: 21 is substituted. In some embodiments, one or more amino acids of an amino acid sequence encoding one or more variable heavy chain CDRs of SEQ ID NO: 16 is substituted. In some embodiments, one or more amino acids of an amino acid sequence encoding one or more variable light chain CDRs of SEQ ID NO: 21 is substituted. In some embodiments, one or more amino acids of an amino acid sequence encoding one or more CDRs of SEQ ID NO: 20 or SEQ ID NO: 25 is substituted. In some embodiments, one or more amino acids of an amino acid sequence encoding one or more variable heavy chain CDRs of SEQ ID NO: 20 is substituted. In some embodiments, one or more amino acids of an amino acid sequence encoding one or more variable light chain CDRs of SEQ ID NO: 25 is substituted.

In some embodiments the monoclonal antibody is a scFv antibody comprising polypeptide chain comprising a variable heavy chain (VH) domain of SEQ ID NO: 16, a linker (e.g., but not limited to, SEQ ID NO: 26), and a variable light chain (VL) domain of SEQ ID NO: 21.

In some embodiments the monoclonal antibody is a scFv-Fc antibody comprising a first and second chain that associate together, each chain comprising a variable heavy chain (VH) domain of SEQ ID NO: 16, a linker (e.g., but not limited to SEQ ID NO: 26), a variable light chain (VL) domain of SEQ ID NO: 21, and an Fc domain. In some embodiments, the Fc domain comprises the Fc domain of SEQ ID NO: 20. In some embodiments, the Fc domain comprises the Fc domain of a mouse IgG1, IgG2, IgG3, or IgG4 constant region. In some embodiments, the Fc domain comprises the Fc domain of a mouse IgG1 constant region. In some embodiments, the Fc domain comprises the Fc domain of a human IgG1, IgG2, IgG3, or IgG4 constant region. In some embodiments, the Fc domain comprises the Fc domain of a human IgG1 constant region.

In some embodiments the monoclonal antibody is a scFv-Fc-scFv antibody comprising a first and second chain that associate together, each chain comprising a first variable heavy chain (VH) domain of SEQ ID NO: 16, a first linker (e.g., but not limited to SEQ ID NO: 26), a first variable light chain (VL) domain of SEQ ID NO: 21, an Fc domain, a second variable heavy chain (VH) domain of SEQ ID NO: 16, a second linker (e.g., but not limited to SEQ ID NO: 26), and a second variable light chain (VL) domain of SEQ ID NO: 21. In some embodiments, the Fc domain comprises the Fc domain of SEQ ID NO: 20. In some embodiments, the Fc domain comprises the Fc domain of a mouse IgG1, IgG2, IgG3, or IgG4 constant region. In some embodiments, the Fc domain comprises the Fc domain of a mouse IgG1 constant region. In some embodiments, the Fc domain comprises the Fc domain of a human IgG1, IgG2, IgG3, or IgG4 constant region. In some embodiments, the Fc domain comprises the Fc domain of a human IgG1 constant region.

The antibody comprising a dimer of the heterodimer comprising SEQ ID NO: 20 and SEQ ID NO: 25 is represented by antibody “8D04A” in embodiments described and depicted in this disclosure.

In some embodiments, the anti-PAG antibodies described herein are chimeric antibodies. As described above, a chimeric antibody can comprise the VH and VL domain of mouse antibodies described herein (e.g. SEQ ID NOs: 5, 11, 16, and 21) and constant domains from human antibodies.

In some embodiments, the anti-PAG antibodies described herein are humanized antibodies. In some embodiments, the amino acid sequence of the anti-PAG antibody heavy chain comprises a VH domain comprising a humanized version of SEQ ID NO: 5. In some embodiments, the amino acid sequence of the anti-PAG antibody light chain comprises a VH domain comprising a humanized version of SEQ ID NO: 11. In some embodiments, the amino acid sequence of the anti-PAG antibody heavy chain comprises a VH domain comprising a humanized version of SEQ ID NO: 16. In some embodiments, the amino acid sequence of the anti-PAG antibody light chain comprises a VH domain comprising a humanized version of SEQ ID NO: 21. Any method of humanizing an antibody known in the art can be used. For example, the CDRs of the VH and VL domains can be inserted into any desired human antibody scaffold or acceptor sequence.

In some embodiments, the amino acid sequence of the anti-PAG antibody VH domain comprises SEQ ID NO: 27. Bolded amino acids represent the CDR-1 (GYTFTSYV (SEQ ID NO:6)), CDR-2 (TYPYNDGT (SEQ ID NO:7)), and CDR-3 (ARYKYGQGFAY (SEQ ID NO:8)), respectively. QVQLVQSGAEVKKPGASVKVSCKASGYTFTSYVMHWVRQAPGQGLEWMGYIYPY NDGTKYNEKFKGRVTMTRDTSTSTVYMELSSLRSEDTAVYYCARYKYGQGFAYW GQGTTVTVSS (SEQ ID NO: 27). In some embodiments, the amino acid sequence of the anti-PAG antibody heavy chain comprises a signal peptide (for example, but not limited to, SEQ ID NO:9) immediately followed by SEQ ID NO: 27. In some embodiments, the signal peptide is cleaved during post-translational modifications that occur in vitro or in vivo. In some embodiments, a nucleic acid sequence encoding the amino acid sequence of the anti-PAG antibody heavy chain comprises a nucleic acid sequence encoding a signal peptide (for example, but not limited to, SEQ ID NO:9) immediately followed by a nucleic acid sequence encoding SEQ ID NO: 27. In some embodiments, the anti-PAG antibody comprises a polypeptide heavy chain comprising a variable heavy chain (VH) domain comprising SEQ ID NO: 27 wherein the CDRs indicated above (SEQ ID NOs: 6, 7, and 8) are replaced with CDR of SEQ ID NOs: 17, 18, and 19.

In some embodiments, the anti-PAG antibody comprises a polypeptide heavy chain comprising a variable heavy chain (VH) domain comprising SEQ ID NO: 27 followed by a constant region comprising a CH1 domain, a hinge domain, a Fc domain (CH2-CH3). In some embodiments the constant region comprises a CH1 domain, a hinge domain, and a Fc domain (CH2-CH3) of a human IgG1, IgG2, IgG3, or IgG4 constant region. In some embodiments the constant region comprises a CH1 domain, a hinge domain, and a Fc domain (CH2-CH3) of a human IgG1 constant region.

In some embodiments, the anti-PAG antibody comprises a polypeptide heavy chain comprising a variable heavy chain (VH) domain comprising SEQ ID NO: 27, wherein the CDRs indicated above (SEQ ID NOs: 6, 7, and 8) are replaced with CDR of SEQ ID NOs: 17, 18, and 19, followed by a constant region comprising a CH1 domain, a hinge domain, a Fc domain (CH2-CH3). In some embodiments the constant region comprises a CH1 domain, a hinge domain, and a Fc domain (CH2-CH3) of a human IgG1, IgG2, IgG3, or IgG4 constant region. In some embodiments the constant region comprises a CH1 domain, a hinge domain, and a Fc domain (CH2-CH3) of a human IgG1 constant region.

In some embodiments, the amino acid sequence of the anti-PAG antibody VL domain comprises SEQ ID NO: 28 or SEQ ID NO: 33. Bolded amino acids represent the CDR-1 (ENIYSN (SEQ ID NO:12)), CDR-2 (AAT (SEQ ID NO:13), and CDR-3 (QHFWGTPWT (SEQ ID NO:14), respectively. DIQMTQSPSSVSASVGDRVTITCRASENIYSNLAWYQQKPGKAPKLLIYAATNLADG VPSRFSGSGSGTDFTLTISSLOPEDFATYYCQHFWGTPWTFGGGTKLEIKRLEPKSC DKT (SEQ ID NO: 28). DIQMTQSPSSVSASVGDRVTITCRASENIYSNLAWYQQKPGKAPKLLIYAATNLADG VPSRFSGSGSGTDFTLTISSLQPEDFATYYCQHFWGTPWTFGGGTKLEIKR (SEQ ID NO: 33) In some embodiments, the amino acid sequence of the anti-PAG antibody light chain comprises a signal peptide (for example, but not limited to, SEQ ID NO:9) immediately followed by SEQ ID NO: 28. In some embodiments, the signal peptide is cleaved during post-translational modifications that occur in vitro or in vivo. In some embodiments, a nucleic acid sequence encoding the amino acid sequence of the anti-PAG antibody light chain comprises a nucleic acid sequence encoding a signal peptide (for example, but not limited to, SEQ ID NO:9) immediately followed by a nucleic acid sequence encoding SEQ ID NO: 28. In some embodiments, the anti-PAG antibody comprises a polypeptide light chain comprising a variable heavy chain (VL) domain comprising SEQ ID NO: 28 wherein the CDRs indicated above (SEQ ID NOs: 12, 13, and 14) are replaced with CDR of SEQ ID NOs: 22, 23, and 24.

In some embodiments, the amino acid sequence of the anti-PAG antibody light chain comprises a signal peptide (for example, but not limited to, SEQ ID NO:9) immediately followed by SEQ ID NO: 33. In some embodiments, the signal peptide is cleaved during post-translational modifications that occur in vitro or in vivo. In some embodiments, a nucleic acid sequence encoding the amino acid sequence of the anti-PAG antibody light chain comprises a nucleic acid sequence encoding a signal peptide (for example, but not limited to, SEQ ID NO:9) immediately followed by a nucleic acid sequence encoding SEQ ID NO: 33. In some embodiments, the anti-PAG antibody comprises a polypeptide light chain comprising a variable heavy chain (VL) domain comprising SEQ ID NO: 33 wherein the CDRs indicated above (SEQ ID NOs: 12, 13, and 14) are replaced with CDR of SEQ ID NOs: 22, 23, and 24.

In some embodiments, the anti-PAG antibody comprises a polypeptide light chain comprising a variable light chain (VL) domain comprising SEQ ID NO: 28 followed by a constant region comprising a CL domain. In some embodiments the constant region comprises a CL domain of a mouse Ig kappa chain or Ig lambda chain. In some embodiments the constant region comprises a CL of a mouse Ig kappa chain. In some embodiments the constant region comprises a CL domain of a human Ig kappa chain or Ig lambda chain. In some embodiments the constant region comprises a CL of a human Ig kappa chain.

In some embodiments, the anti-PAG antibody comprises a polypeptide light chain comprising a variable light chain (VL) domain comprising SEQ ID NO: 33 followed by a constant region comprising a CL domain. In some embodiments the constant region comprises a CL domain of a mouse Ig kappa chain or Ig lambda chain. In some embodiments the constant region comprises a CL of a mouse Ig kappa chain. In some embodiments the constant region comprises a CL domain of a human Ig kappa chain or Ig lambda chain. In some embodiments the constant region comprises a CL of a human Ig kappa chain.

In some embodiments, the anti-PAG antibody comprises a polypeptide light chain comprising a variable light chain (VL) domain comprising SEQ ID NO: 28, wherein the CDRs indicated above (SEQ ID NOs: 12, 13, and 14) are replaced with CDR of SEQ ID NOs: 22, 23, and 24, followed by a constant region comprising a CL domain. In some embodiments the constant region comprises a CL domain of a mouse Ig kappa chain or Ig lambda chain. In some embodiments the constant region comprises a CL of a mouse Ig kappa chain. In some embodiments the constant region comprises a CL domain of a human Ig kappa chain or Ig lambda chain. In some embodiments the constant region comprises a CL of a human Ig kappa chain.

In some embodiments, the anti-PAG antibody comprises a polypeptide light chain comprising a variable light chain (VL) domain comprising SEQ ID NO: 33, wherein the CDRs indicated above (SEQ ID NOs: 12, 13, and 14) are replaced with CDR of SEQ ID NOs: 22, 23, and 24, followed by a constant region comprising a CL domain. In some embodiments the constant region comprises a CL domain of a mouse Ig kappa chain or Ig lambda chain. In some embodiments the constant region comprises a CL of a mouse Ig kappa chain. In some embodiments the constant region comprises a CL domain of a human Ig kappa chain or Ig lambda chain. In some embodiments the constant region comprises a CL of a human Ig kappa chain.

In some embodiments, the monoclonal antibody comprises a first and third chain each comprising SEQ ID NO: 27 and a second and fourth chains each comprising SEQ ID NO: 28. In some embodiments, the monoclonal antibody comprises a first and third chain each comprising SEQ ID NO: 27 followed by a constant region comprising a CH1 domain, a hinge domain, a Fc domain (CH2-CH3) and a second and fourth chains each comprising SEQ ID NO: 28 followed by a constant region comprising a CL domain, wherein the constant region of the first and third chain comprises a CH1 domain, a hinge domain, and a Fc domain (CH2-CH3) of a human IgG1, IgG2, IgG3, or IgG4 constant region and the constant region of the second and fourth chain comprises a CL domain of a human Ig kappa chain or Ig lambda chain. In some embodiments, the first and second chains are linked by one or more covalent disulfide bonds and the third and fourth chains are linked by one or more covalent disulfide bonds. In some embodiments, the first and third chains are linked by one or more disulfide bonds.

In some embodiments, the monoclonal antibody comprises a first and third chain each comprising SEQ ID NO: 27 and a second and fourth chains each comprising SEQ ID NO: 33. In some embodiments, the monoclonal antibody comprises a first and third chain each comprising SEQ ID NO: 27 followed by a constant region comprising a CH1 domain, a hinge domain, a Fc domain (CH2-CH3) and a second and fourth chains each comprising SEQ ID NO: 33 followed by a constant region comprising a CL domain, wherein the constant region of the first and third chain comprises a CH1 domain, a hinge domain, and a Fc domain (CH2-CH3) of a human IgG1, IgG2, IgG3, or IgG4 constant region and the constant region of the second and fourth chain comprises a CL domain of a human Ig kappa chain or Ig lambda chain. In some embodiments, the first and second chains are linked by one or more covalent disulfide bonds and the third and fourth chains are linked by one or more covalent disulfide bonds. In some embodiments, the first and third chains are linked by one or more disulfide bonds.

In some embodiments, the anti-PAG antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 27. In some embodiments, the anti-PAG antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 28. In some embodiments, the anti-PAG antibody comprises an amino acid sequence comprising the CDRs of the variable heavy chain domain and the CDRs of the variable light chain domain of the anti-PAG antibody, wherein the CDR sequences are SEQ ID NO: 6 (CDR-H1), SEQ ID NO: 7 (CDR-H2), SEQ ID NO: 8 (CDR-H3), SEQ ID NO: 12 (CDR-L1), SEQ ID NO: 13 (CDR-L2), and SEQ ID NO: 14 (CDR-L3), or are SEQ ID NO: 17 (CDR-H1), SEQ ID NO: 18 (CDR-H2), SEQ ID NO: 19 (CDR-H3), SEQ ID NO: 22 (CDR-L1), SEQ ID NO: 23 (CDR-L2), and SEQ ID NO: 24 (CDR-L3) and wherein the framework regions (FRs) of the variable heavy chain domain and the FRs of the variable light chain domain of the anti-PAG antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 27 and SEQ ID NO: 28, respectively.

In some embodiments, the anti-PAG antibody heavy chain comprises an amino acid sequence 80, 81, 82, 83, 84, 85, 86, 87, 88, 89, 90, 91, 92, 93, 94, 95, 96, 97, 98 or 99% identical to SEQ ID NO: 27. In some embodiments, the anti-PAG antibody light chain comprises an amino acid sequence 80, 81, 82, 83, 84, 85, 86, 87, 88, 89, 90, 91, 92, 93, 94, 95, 96, 97, 98 or 99% identical to SEQ ID NO: 33. In some embodiments, the anti-PAG antibody comprises an amino acid sequence comprising the CDRs of the variable heavy chain domain and the CDRs of the variable light chain domain of the anti-PAG antibody, wherein the CDR sequences are SEQ ID NO: 6 (CDR-H1), SEQ ID NO: 7 (CDR-H2), SEQ ID NO: 8 (CDR-H3), SEQ ID NO: 12 (CDR-L1), SEQ ID NO: 13 (CDR-L2), and SEQ ID NO: 14 (CDR-L3), or arc SEQ ID NO: 17 (CDR-H1), SEQ ID NO: 18 (CDR-H2), SEQ ID NO: 19 (CDR-H3), SEQ ID NO: 22 (CDR-L1), SEQ ID NO: 23 (CDR-L2), and SEQ ID NO: 24 (CDR-L3) and wherein the framework regions (FRs) of the variable heavy chain domain and the FRs of the variable light chain domain of the anti-PAG antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 27 and SEQ ID NO: 33, respectively.

In some embodiments, one or more amino acids of an amino acid sequence encoding one or more CDRs of SEQ ID NO: 27 or SEQ ID NO: 28 is substituted. In some embodiments, one or more amino acids of an amino acid sequence encoding one or more variable heavy chain CDRs of SEQ ID NO: 27 is substituted. In some embodiments, one or more amino acids of an amino acid sequence encoding one or more variable light chain CDRs of SEQ ID NO: 28 is substituted.

In some embodiments, one or more amino acids of an amino acid sequence encoding one or more CDRs of SEQ ID NO: 27 or SEQ ID NO: 33 is substituted. In some embodiments, one or more amino acids of an amino acid sequence encoding one or more variable heavy chain CDRs of SEQ ID NO: 27 is substituted. In some embodiments, one or more amino acids of an amino acid sequence encoding one or more variable light chain CDRs of SEQ ID NO: 33 is substituted.

In some embodiments the monoclonal antibody is a scFv antibody comprising polypeptide chain comprising a variable heavy chain (VH) domain of SEQ ID NO: 27, a linker (e.g., but not limited to SEQ ID NO:26), and a variable light chain (VL) domain of SEQ ID NO: 28.

In some embodiments the monoclonal antibody is a scFv antibody comprising polypeptide chain comprising a variable heavy chain (VH) domain of SEQ ID NO: 27, a linker (e.g., but not limited to SEQ ID NO:26), and a variable light chain (VL) domain of SEQ ID NO: 33.

In some embodiments the monoclonal antibody is a scFv antibody comprising polypeptide chain comprising a variable heavy chain (VH) domain of SEQ ID NO: 27 wherein the CDRs indicated above (SEQ ID NOs: 6, 7, and 8) are replaced with CDR of SEQ ID NOs: 17, 18, and 19, a linker (e.g., but not limited to SEQ ID NO:26), and a variable light chain (VL) domain of SEQ ID NO: 28 wherein the CDRs indicated above (SEQ ID NOs: 12, 13, and 14) are replaced with CDR of SEQ ID NOs: 22, 23, and 24.

In some embodiments the monoclonal antibody is a scFv antibody comprising polypeptide chain comprising a variable heavy chain (VH) domain of SEQ ID NO: 27 wherein the CDRs indicated above (SEQ ID NOs: 6, 7, and 8) are replaced with CDR of SEQ ID NOs: 17, 18, and 19, a linker (e.g., but not limited to SEQ ID NO:26), and a variable light chain (VL) domain of SEQ ID NO: 33 wherein the CDRs indicated above (SEQ ID NOs: 12, 13, and 14) are replaced with CDR of SEQ ID NOs: 22, 23, and 24.

In some embodiments the monoclonal antibody is a scFv-Fc antibody comprising a first and second chain that associate together, each chain comprising a variable heavy chain (VH) domain of SEQ ID NO: 27, a linker (e.g., but not limited to SEQ ID NO: 26), a variable light chain (VL) domain of SEQ ID NO: 28, and an Fc domain. In some embodiments, the Fc domain comprises the Fc domain of a human IgG1, IgG2, IgG3, or IgG4 constant region. In some embodiments, the Fc domain comprises the Fc domain of a human IgG1 constant region.

In some embodiments the monoclonal antibody is a scFv-Fc antibody comprising a first and second chain that associate together, each chain comprising a variable heavy chain (VH) domain of SEQ ID NO: 27, a linker (e.g., but not limited to SEQ ID NO: 26), a variable light chain (VL) domain of SEQ ID NO: 33, and an Fc domain. In some embodiments, the Fc domain comprises the Fc domain of a human IgG1, IgG2, IgG3, or IgG4 constant region. In some embodiments, the Fc domain comprises the Fc domain of a human IgG1 constant region.

In some embodiments the monoclonal antibody is a scFv-Fc antibody comprising a first and second chain that associate together, each chain comprising a variable heavy chain (VH) domain of SEQ ID NO: 27 wherein the CDRs indicated above (SEQ ID NOs: 6, 7, and 8) are replaced with CDR of SEQ ID NOs: 17, 18, and 19, a linker (e.g., but not limited to SEQ ID NO: 26), a variable light chain (VL) domain of SEQ ID NO: 28 wherein the CDRs indicated above (SEQ ID NOs: 12, 13, and 14) are replaced with CDR of SEQ ID NOs: 22, 23, and 24, and an Fc domain. In some embodiments, the Fc domain comprises the Fc domain of a human IgG1, IgG2, IgG3, or IgG4 constant region. In some embodiments, the Fc domain comprises the Fc domain of a human IgG1 constant region.

In some embodiments the monoclonal antibody is a scFv-Fc-scFv antibody comprising a first and second chain that associate together, each chain comprising a first variable heavy chain (VH) domain of SEQ ID NO: 27, a first linker (e.g., but not limited to SEQ ID NO: 26), a first variable light chain (VL) domain of SEQ ID NO: 28, an Fc domain, a second variable heavy chain (VH) domain of SEQ ID NO: 27, a second linker (e.g., but not limited to SEQ ID NO: 26), and a second variable light chain (VL) domain of SEQ ID NO: 28. In some embodiments, the Fc domain comprises the Fc domain of a human IgG1, IgG2, IgG3, or IgG4 constant region. In some embodiments, the Fc domain comprises the Fc domain of a human IgG1 constant region.

In some embodiments the monoclonal antibody is a scFv-Fc-scFv antibody comprising a first and second chain that associate together, each chain comprising a first variable heavy chain (VH) domain of SEQ ID NO: 27, a first linker (e.g., but not limited to SEQ ID NO: 26), a first variable light chain (VL) domain of SEQ ID NO: 33, an Fc domain, a second variable heavy chain (VH) domain of SEQ ID NO: 27, a second linker (e.g., but not limited to SEQ ID NO: 26), and a second variable light chain (VL) domain of SEQ ID NO: 28. In some embodiments, the Fc domain comprises the Fc domain of a human IgG1, IgG2, IgG3, or IgG4 constant region. In some embodiments, the Fc domain comprises the Fc domain of a human IgG1 constant region.

In some embodiments the monoclonal antibody is a scFv-Fc-scFv antibody comprising a first and second chain that associate together, each chain comprising a first variable heavy chain (VH) domain of SEQ ID NO: 27 wherein the CDRs indicated above (SEQ ID NOs: 6, 7, and 8) are replaced with CDR of SEQ ID NOs: 17, 18, and 19, a first linker (e.g., but not limited to SEQ ID NO: 26), a first variable light chain (VL) domain of SEQ ID NO: 28 wherein the CDRs indicated above (SEQ ID NOs: 12, 13, and 14) are replaced with CDR of SEQ ID NOs: 22, 23, and 24, an Fc domain, a second variable heavy chain (VH) domain of SEQ ID NO: 27 wherein the CDRs indicated above (SEQ ID NOs: 6, 7, and 8) are replaced with CDR of SEQ ID NOs: 17, 18, and 19, a second linker (e.g., but not limited to SEQ ID NO: 26), and a second variable light chain (VL) domain of SEQ ID NO: 28 wherein the CDRs indicated above (SEQ ID NOs: 12, 13, and 14) are replaced with CDR of SEQ ID NOs: 22, 23, and 24. In some embodiments, the Fc domain comprises the Fc domain of a human IgG1, IgG2, IgG3, or IgG4 constant region. In some embodiments, the Fc domain comprises the Fc domain of a human IgG1 constant region.

In some embodiments the monoclonal antibody is a scFv-Fc-scFv antibody comprising a first and second chain that associate together, each chain comprising a first variable heavy chain (VH) domain of SEQ ID NO: 27 wherein the CDRs indicated above (SEQ ID NOs: 6, 7, and 8) are replaced with CDR of SEQ ID NOs: 17, 18, and 19, a first linker (e.g., but not limited to SEQ ID NO: 26), a first variable light chain (VL) domain of SEQ ID NO: 33 wherein the CDRs indicated above (SEQ ID NOs: 12, 13, and 14) are replaced with CDR of SEQ ID NOs: 22, 23, and 24, an Fc domain, a second variable heavy chain (VH) domain of SEQ ID NO: 27 wherein the CDRs indicated above (SEQ ID NOs: 6, 7, and 8) are replaced with CDR of SEQ ID NOs: 17, 18, and 19, a second linker (e.g., but not limited to SEQ ID NO: 26), and a second variable light chain (VL) domain of SEQ ID NO: 28 wherein the CDRs indicated above (SEQ ID NOs: 12, 13, and 14) are replaced with CDR of SEQ ID NOs: 22, 23, and 24. In some embodiments, the Fc domain comprises the Fc domain of a human IgG1, IgG2, IgG3, or IgG4 constant region. In some embodiments, the Fc domain comprises the Fc domain of a human IgG1 constant region.

In certain aspects, described herein is an antibody comprising a dimer of the heterodimer comprising SEQ ID NO: 27 and SEQ ID NO: 28. In certain aspects, described herein is an antibody comprising a dimer of the heterodimer comprising SEQ ID NO: 27 and SEQ ID NO: 33.

In some embodiments, the molecular three-dimensional structure of an anti-PAG antibody can be predicted based on X-ray crystallography, and/or cryo-EM, and/or using structure prediction algorithms (e.g., machine learning algorithms) known in the art, such as AlphaFold or RaptorX. In some embodiments, the structure prediction algorithm is a computational method that is used to predict three-dimensional (3D) antibody structures based on a given nucleic acid or amino acid sequence. In some embodiments, the structure prediction algorithm predicts the 3D coordinates of all heavy atoms for a given antibody using a nucleic acid or amino acid sequence and/or aligned sequences of homologues as inputs. In some embodiments, the structure of an anti-PAG antibody is predicted using a combination of methods, e.g., using a combination of AlphaFold (or any other structure prediction algorithm known in the art) and X-ray crystallography or cryo-EM. In some embodiments, the structure prediction is improved by combining the use of AlphaFold (or any other structure prediction algorithm known in the art) and X-ray crystallography or cryo-EM. In some embodiments, the structure of an anti-PAG antibody is predicted by using a computational structure prediction algorithm (e.g., AlphaFold or RaptorX) and the structure prediction of the anti-PAG or the anti-PAG antibody is then refined by using X-ray crystallography or cryo-EM. In some embodiments, the anti-PAG antibody comprises a three-dimensional structure that is similar to the three-dimensional structure of anti-PAG that comprises SEQ ID NOs: 5 and 11, 16 and 21, 27 and 28, or 27 and 33.

In some embodiments, the structure prediction algorithm can be used to model the structure of a first antibody (e.g., an anti-PAG antibody) (e.g., a reference an anti-PAG antibody comprising SEQ ID NOs: 5, 11, or a combination thereof, SEQ ID NOs: 16, 21, or a combination thereof, or SEQ ID NOs: 27, 28, 33, or a combination thereof) and compare a predicted structure of second antibody (e.g., an anti-PAG antibody) against the predicted structure of the first antibody such that the second antibody can be categorized in the same class as the first antibody based on its structural similarity to the first antibody. In some embodiments, a metric of structural similarity between two antibodies can be obtained based on the output of a structure prediction algorithm known in the art. In some embodiments, the metric of structural similarity between two antibodies is based on a similarity distance.

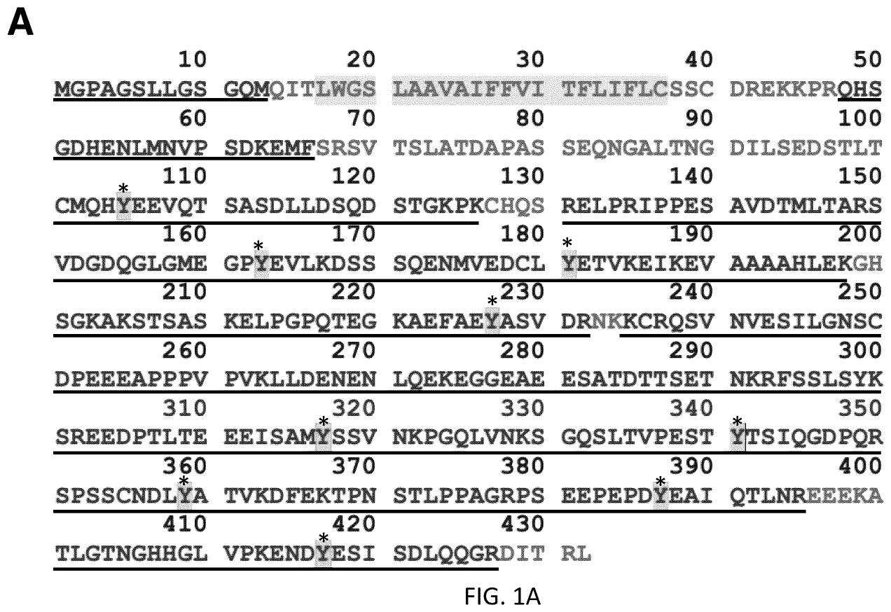

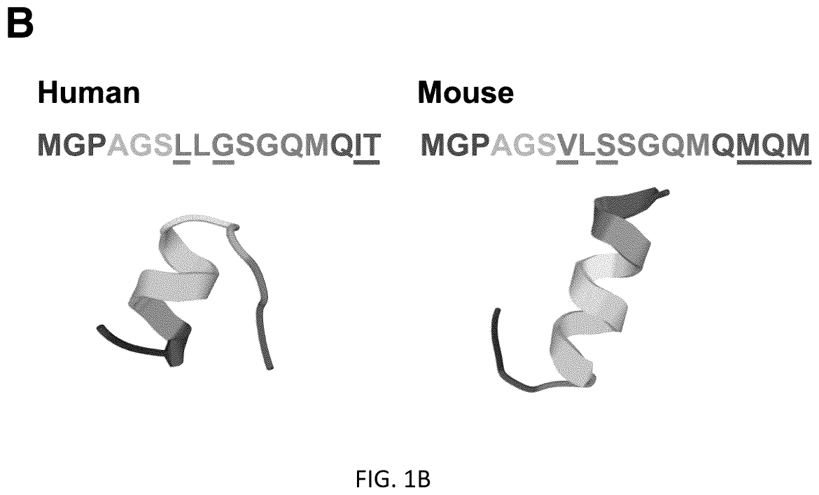

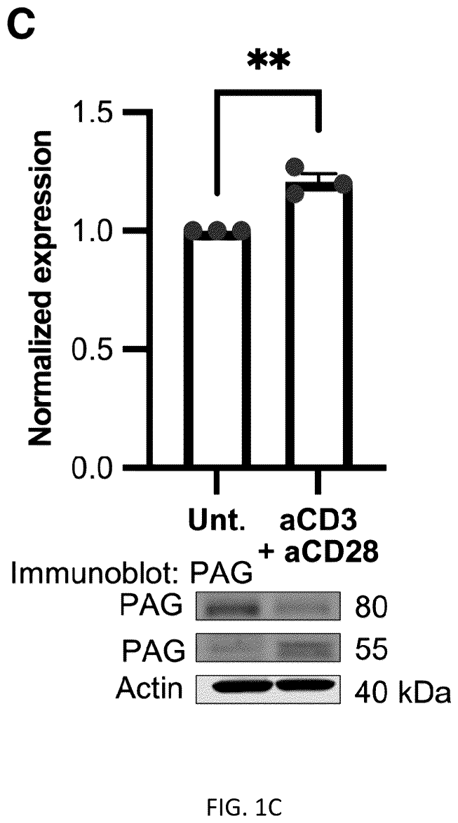

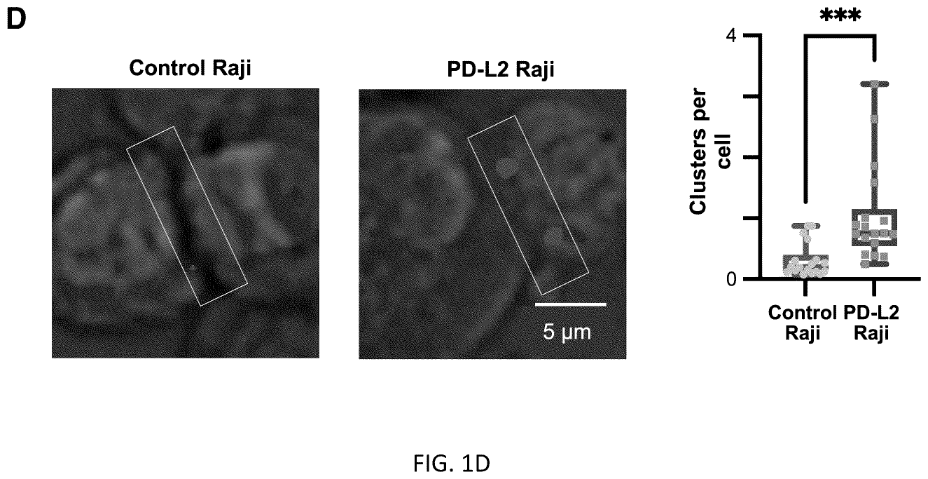

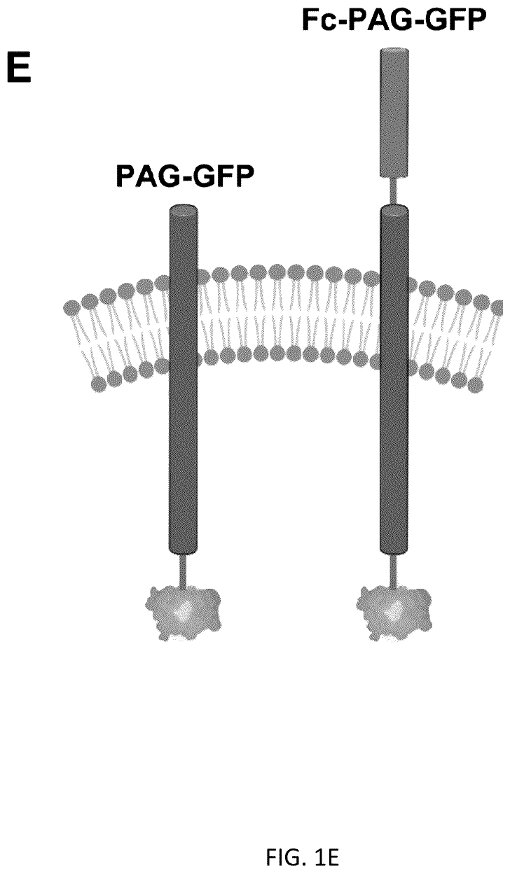

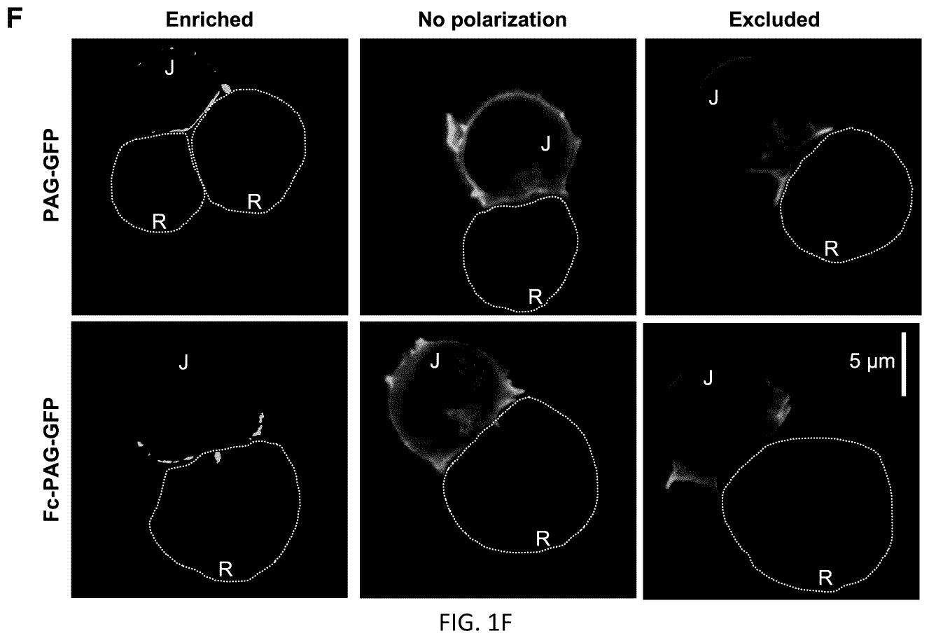

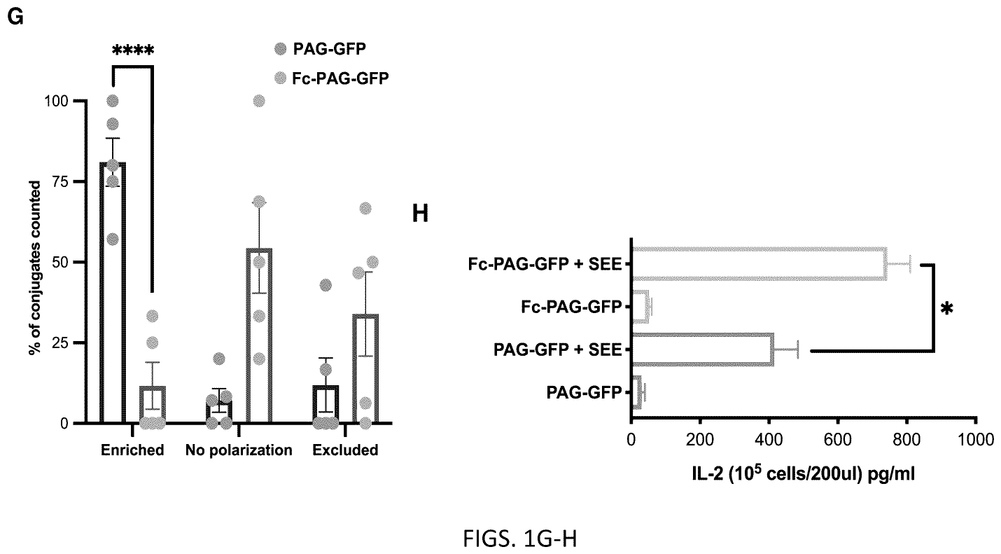

In some embodiments, the structure of the anti-PAG antibody allows the anti-PAG antibody to bind to PAG. In some embodiments, the structure of the anti-PAG antibody allows the anti-PAG antibody to bind to the extracellular portion of human PAG provided in FIG. 1B. In some embodiments, disclosed herein is a new class of anti-PAG antibodies which comprise structural similarity to one another such that the new class of anti-PAG antibodies are capable of binding to the extracellular portion of human PAG provided in FIG. 1B. In some embodiments, the anti-PAG antibody comprises means for binding the extracellular portion of human PAG provided in FIG. 1B. In some embodiments, means for binding PAG comprises an anti-PAG antibody that comprises SEQ ID NOs: 5 and 11, SEQ ID NOs: 16 and 21, SEQ ID NOS: 27 and 28, SEQ ID NOS: 27 and 33.

### Nucleic Acid Sequences of Anti-PAG Antibodies

A person skilled in the art can identify the nucleic acid sequences encoding the features identified in the corresponding amino acid sequences (e.g., CDRs, variable heavy domain, variable light domain, CH1 domain, hinge domain, Fc domain, and any additional Fc domain) by translating the nucleic acid sequences into amino acid sequences. In some embodiments, the nucleic acid sequences may be codon optimized.

In some embodiments, the nucleic acid sequence of the anti-PAG antibody comprises a nucleic acid sequence encoding a heavy chain amino acid sequence comprising SEQ ID NO: 5.

In some embodiments, the nucleic acid sequence encoding SEQ ID NO: 5 comprises SEQ ID NO: 29. GAGGTCCAGC TGCAGCAGTC TGGACCTGAA CTGGTAAAGC CTGGGGCTTC AGTGAAGATG TCCTGCAAGG CTTCTGGATA CACATTCACT AGCTATGTTA TGCACTGGGT GAAGCAGAAG CCTGGGCAGG GCCTTGAGTG GATTGGATAT ATTTATCCTT ACAATGATGG TACTAAGTAC AATGAGAAGT TCAAAGGCAA GGCCACACTG ACTTCAGACA AATCCTCCAG CACAGCCTAC ATGGAGCTCA GCAGCCTGAC CTCTGAGGAC TCTGCGGTCT ATTATTGTGC AAGATATAAG TATGGTCAGG GGTTTGCTTA CTGGGGCCAA GGGACTCGGG TCACTGTCTC TGCA (SEQ ID NO: 29). Nucleic acid sequences encoding the respective CDRs in SEQ ID NO:5 can be determined from the amino acid sequence of SEQ ID NO: 5 as identified herein by a person of skill in the art. In some embodiments, the nucleic acid sequence encoding the anti-PAG antibody heavy chain comprises a nucleic acid sequence encoding SEQ ID NO: 9 immediately followed by a nucleic acid sequence encoding SEQ ID NO: 5 (e.g., but not limited to SEQ ID NO:29). In some embodiments, the signal peptide is cleaved during post-translational modifications that occur in vitro or in vivo. In some embodiments, the nucleic acid sequence of the anti-PAG antibody comprises a nucleic acid sequence encoding a light chain amino acid sequence comprising SEQ ID NO: 11.

In some embodiments, the nucleic acid sequence encoding SEQ ID NO: 11 comprises SEQ ID NO: 30. GACATCCAGA TGACTCAGTC TCCAGCCTCC CTATCTGTAT CTGTGGGAGA AACTGTCACC ATCACATGTC GAGCAAGTGA GAATATTTAC AGTAATTTAG CATGGTATCA GCAGAAACAG GGAAAATCTC CTCAGCTCCT GGTCTATGCT GCAACAAACT TAGCAGATGG TGTGCCATCA AGGTTCAGTG GCAGTGGATC AGGCACACAG TATTCCCTCA AGATCAACAG CCTGCAGTCT GAAGATTTTG GGAGTTATTA CTGTCAACAT TTTTGGGGTA CTCCGTGGAC GTTCGGTGGA GGCACCAAGC TGGAAATCAA A (SEQ ID NO: 30). Nucleic acid sequences encoding the respective CDRs in SEQ ID NO: 11 can be determined from the amino acid sequence of SEQ ID NO: 11 as identified herein by a person of skill in the art. In some embodiments, the nucleic acid sequence encoding the anti-PAG antibody light chain comprises a nucleic acid sequence encoding SEQ ID NO: 9 immediately followed by a nucleic acid sequence encoding SEQ ID NO: 11 (e.g., but not limited to SEQ ID NO: 30). In some embodiments, the signal peptide is cleaved during post-translational modifications that occur in vitro or in vivo.

In some embodiments, the nucleic acid sequence encoding the anti-PAG antibody heavy chain comprises a nucleic acid sequence encoding SEQ ID NO: 10. In some embodiments, the nucleic acid sequence encoding the anti-PAG antibody heavy chain comprises a nucleic acid sequence encoding SEQ ID NO: 9 immediately followed by a nucleic acid sequence encoding SEQ ID NO: 10. In some embodiments, the signal peptide is cleaved during post-translational modifications that occur in vitro or in vivo. Nucleic acid sequences encoding the anti-PAG antibody heavy chain and respective CDRs, VH domain, CH1 domain, hinge domain, and Fc domain can be determined from the amino acid sequence of SEQ ID NO: 10 by a person of skill in the art.

In some embodiments, the nucleic acid sequence encoding the anti-PAG antibody light chain comprises a nucleic acid sequence encoding SEQ ID NO: 15. In some embodiments, the nucleic acid sequence encoding the anti-PAG antibody light chain comprises a nucleic acid sequence encoding SEQ ID NO: 9 immediately followed by a nucleic acid sequence encoding SEQ ID NO: 15. In some embodiments, the signal peptide is cleaved during post-translational modifications that occur in vitro or in vivo. Nucleic acid sequences encoding the anti-PAG antibody light chain and respective CDRs, VL domain, and CL domain can be determined from the amino acid sequence of SEQ ID NO: 15 by a person of skill in the art.

In some embodiments, the nucleic acid sequence encoding an anti-PAG heavy chain comprise a nucleic acid sequence 80, 81, 82, 83, 84, 85, 86, 87, 88, 89, 90, 91, 92, 93, 94, 95, 96, 97, 98 or 99% identical to SEQ ID NO: 29. In some embodiments, the nucleic acid sequence encoding an anti-PAG light chain comprise a nucleic acid sequence 80, 81, 82, 83, 84, 85, 86, 87, 88, 89, 90, 91, 92, 93, 94, 95, 96, 97, 98 or 99% identical to SEQ ID NO: 30. In some embodiments, the nucleic acid sequence encoding framework regions (FRs) of the variable heavy chain domain and the FRs of the variable light chain domain of the anti-PAG antibody comprise a nucleic acid sequence 80, 81, 82, 83, 84, 85, 86, 87, 88, 89, 90, 91, 92, 93, 94, 95, 96, 97, 98 or 99% identical to the sequence encoding the FRs of SEQ ID NO: 29 and SEQ ID NO: 30.

In some embodiments, one or more nucleic acids of a nucleic acid sequence encoding one or more CDRs of SEQ ID NO: 29 or SEQ ID NO: 30 is substituted. In some embodiments, one or more nucleic acids of a nucleic acid sequence encoding one or more variable heavy chain CDRs of SEQ ID NO: 29 is substituted. In some embodiments, one or more nucleic acids of a nucleic acid sequence encoding one or more variable light chain CDRs of SEQ ID NO: 30 is substituted.

In some embodiments, the nucleic acid sequence of the anti-PAG antibody comprises a nucleic acid sequence encoding a heavy chain amino acid sequence comprising SEQ ID NO: 16.

In some embodiments, the nucleic acid sequence encoding SEQ ID NO: 16 comprises SEQ ID NO: 31. CAGATCCAGTTGGTGCAGTCTGGACCTGAGCTGAAGAAGCCTGGAGAGACAGTC AAGATCTCCTGCAAGGCTTCTGGGTATATCTTCACAAACTATGGAATGAACTGGG TGAAGCAGGCTCCAGGAAAGGGTTTAAAGTGGATGGGCTGGATAAACCCCTACA CTGGAGAGGCAACATATGATGATGACTTCAAGGGACGGTTTGCCTTCTCTTTGGA AACCTCTGCCAACACTGCCTATTTGCAGATCAACAACCTCAAAAATGAGGACAC GGCTACATATTTCTGTGCAAAGACTGGGACGACTTACTGGGGCCAAGGGACTCT GGTCACTGTCTCTGCA (SEQ ID NO: 31). Nucleic acid sequences encoding the respective CDRs in SEQ ID NO: 16 can be determined from the amino acid sequence of SEQ ID NO: 16 as identified herein by a person of skill in the art. In some embodiments, the nucleic acid sequence encoding the anti-PAG antibody heavy chain comprises a nucleic acid sequence encoding SEQ ID NO: 9 immediately followed by a nucleic acid sequence encoding SEQ ID NO: 16 (e.g., but not limited to SEQ ID NO:31). In some embodiments, the signal peptide is cleaved during post-translational modifications that occur in vitro or in vivo.

In some embodiments, the nucleic acid sequence of the anti-PAG antibody comprises a nucleic acid sequence encoding a light chain amino acid sequence comprising SEQ ID NO: 21

In some embodiments, the nucleic acid sequence encoding SEQ ID NO: 21 comprises SEQ ID NO: 32. CAAATTGTTCTCACCCAGTCTCCAGCAATCATGTCTGCATCTCCAGGGGAGAAGG TCACCATGACCTGCAGTGCCAGCTCAAGTGTAAGTTACATGTACTGGTACCAGCA GAAGTCAGGCACCTCCCCCAAAAGATGGATTTATGACACATCCAAACTGGCTTCT GGAGTCCCTGCTCGCTTCAGTGGCAGTGGGTCTGGGACCTCTTACTCTCTCACAA TCAACAACATGGAGGCTGAAGATGCTGCCACTTATTACTGCCAGCAGTGGAGTA GTAACCCGCTCACGTTCGGTGCTGGGACCAAGCTGGAGCTGAAA (SEQ ID NO: 32). Nucleic acid sequences encoding the respective CDRs in SEQ ID NO: 21 can be determined from the amino acid sequence of SEQ ID NO: 21 as identified herein by a person of skill in the art. In some embodiments, the nucleic acid sequence encoding the anti-PAG antibody light chain comprises a nucleic acid sequence encoding SEQ ID NO: 9 immediately followed by a nucleic acid sequence encoding SEQ ID NO: 21 (e.g., but not limited to SEQ ID NO: 32). In some embodiments, the signal peptide is cleaved during post-translational modifications that occur in vitro or in vivo.

In some embodiments, the nucleic acid sequence encoding the anti-PAG antibody heavy chain comprises a nucleic acid sequence encoding SEQ ID NO: 20. In some embodiments, the nucleic acid sequence encoding the anti-PAG antibody heavy chain comprises a nucleic acid sequence encoding SEQ ID NO: 9 immediately followed by a nucleic acid sequence encoding SEQ ID NO: 20. In some embodiments, the signal peptide is cleaved during post-translational modifications that occur in vitro or in vivo. Nucleic acid sequences encoding the anti-PAG antibody heavy chain and respective CDRs, VH domain, CH1 domain, hinge domain, and Fc domain can be determined from the amino acid sequence of SEQ ID NO: 20 by a person of skill in the art.

In some embodiments, the nucleic acid sequence encoding the anti-PAG antibody light chain comprises a nucleic acid sequence encoding SEQ ID NO: 25. In some embodiments, the nucleic acid sequence encoding the anti-PAG antibody light chain comprises a nucleic acid sequence encoding SEQ ID NO: 9 immediately followed by a nucleic acid sequence encoding SEQ ID NO: 25. In some embodiments, the signal peptide is cleaved during post-translational modifications that occur in vitro or in vivo. Nucleic acid sequences encoding the anti-PAG antibody light chain and respective CDRs, VL domain, and CL domain can be determined from the amino acid sequence of SEQ ID NO: 25 by a person of skill in the art.

In some embodiments, the nucleic acid sequence encoding an anti-PAG heavy chain comprise a nucleic acid sequence 80, 81, 82, 83, 84, 85, 86, 87, 88, 89, 90, 91, 92, 93, 94, 95, 96, 97, 98 or 99% identical to SEQ ID NO: 31. In some embodiments, the nucleic acid sequence encoding an anti-PAG light chain comprise a nucleic acid sequence 80, 81, 82, 83, 84, 85, 86, 87, 88, 89, 90, 91, 92, 93, 94, 95, 96, 97, 98 or 99% identical to SEQ ID NO: 32. In some embodiments, the nucleic acid sequence encoding framework regions (FRs) of the variable heavy chain domain and the FRs of the variable light chain domain of the anti-PAG antibody comprise a nucleic acid sequence 80, 81, 82, 83, 84, 85, 86, 87, 88, 89, 90, 91, 92, 93, 94, 95, 96, 97, 98 or 99% identical to the sequence encoding the FRs of SEQ ID NO: 31 and SEQ ID NO: 32.

In some embodiments, one or more nucleic acids of a nucleic acid sequence encoding one or more CDRs of SEQ ID NO: 31 or SEQ ID NO: 32 is substituted. In some embodiments, one or more nucleic acids of a nucleic acid sequence encoding one or more variable heavy chain CDRs of SEQ ID NO: 31 is substituted. In some embodiments, one or more nucleic acids of a nucleic acid sequence encoding one or more variable light chain CDRs of SEQ ID NO: 32 is substituted.

In some embodiments, the nucleic acid sequence of the anti-PAG antibody comprises a nucleic acid sequence encoding a light chain amino acid sequence comprising SEQ ID NO: 27. Nucleic acid sequences encoding the respective CDRs in SEQ ID NO: 27 can be determined from the amino acid sequence of SEQ ID NO: 27 as identified herein by a person of skill in the art. In some embodiments, the nucleic acid sequence encoding the anti-PAG antibody heavy chain comprises a nucleic acid sequence encoding SEQ ID NO: 9 immediately followed by a nucleic acid sequence encoding SEQ ID NO: 27. In some embodiments, the signal peptide is cleaved during post-translational modifications that occur in vitro or in vivo.

In some embodiments, the nucleic acid sequence encoding the anti-PAG antibody light chain comprises a nucleic acid sequence encoding SEQ ID NO: 28. Nucleic acid sequences encoding the respective CDRs in SEQ ID NO: 28 can be determined from the amino acid sequence of SEQ ID NO: 28 as identified herein by a person of skill in the art. In some embodiments, the nucleic acid sequence encoding the anti-PAG antibody light chain comprises a nucleic acid sequence encoding SEQ ID NO: 9 immediately followed by a nucleic acid sequence encoding SEQ ID NO: 28. In some embodiments, the signal peptide is cleaved during post-translational modifications that occur in vitro or in vivo.

In some embodiments, the nucleic acid sequence encoding the anti-PAG antibody light chain comprises a nucleic acid sequence encoding SEQ ID NO: 33. Nucleic acid sequences encoding the respective CDRs in SEQ ID NO: 33 can be determined from the amino acid sequence of SEQ ID NO: 33 as identified herein by a person of skill in the art. In some embodiments, the nucleic acid sequence encoding the anti-PAG antibody light chain comprises a nucleic acid sequence encoding SEQ ID NO: 9 immediately followed by a nucleic acid sequence encoding SEQ ID NO: 33. In some embodiments, the signal peptide is cleaved during post-translational modifications that occur in vitro or in vivo.

In some embodiments, the nucleic acid sequence comprises a nucleic acid sequence encoding any of the scFv, scFv-Fc and scFv-Fc-scFv formats described herein.

### Amino Acid and Nucleic Acid Sequences of Anti-PD-1 Antibodies

In some embodiments, the anti-PAG antibody can be used in combination with an anti-PD-1 antibody including Nivolumab, Pembrolizumab, Cemiplimab, Retifanlimab, Dostarlimab, Zimberclimab, Tisclelizumab, Camrelizumab, Sintilimab, Penpulimab, or any other anti-PD-1 antibody known in the art or an anti-PD-1 antibody that comprises at least a portion of the amino acid sequence encoding the anti-PD-1 targeting portion of Nivolumab, Pembrolizumab, Cemiplimab, Retifanlimab, Dostarlimab, Zimberclimab, Tisclelizumab, Camrelizumab, Sintilimab, Penpulimab, or any other anti-PD-1 antibody known in the art. In some embodiments, the amino acid sequence of the anti-PD-1 antibody comprises at least a variable heavy and variable light chain portions of the amino acid sequence of Nivolumab, Pembrolizumab, Cemiplimab, Retifanlimab, Dostarlimab, Zimberclimab, Tiselelizumab, Camrelizumab, Sintilimab, Penpulimab, or any other anti-PD-1 antibody known in the art. In some embodiments, the amino acid sequence of the anti-PD-1 antibody comprises at least the CDRs of the variable heavy chain and the CDRs of the variable light chain portions of the amino acid sequence of Nivolumab, Pembrolizumab, Cemiplimab, Retifanlimab, Dostarlimab, Zimberclimab, Tisclelizumab, Camrelizumab, Sintilimab, Penpulimab, or any other anti-PD-1 antibody known in the art. In some embodiments, the nucleic acid sequence the anti-PD-1 antibody codes for an amino acid sequence that comprises at least a portion of the amino acid sequence of Nivolumab, Pembrolizumab, Cemiplimab, Retifanlimab, Dostarlimab, Zimberelimab, Tiselelizumab, Camrelizumab, Sintilimab, Penpulimab, or any other anti-PD-1 antibody known in the art. In some embodiments, the nucleic acid sequence of the anti-PD-1 antibody codes for an amino acid sequence that comprises at least a variable heavy and variable light chain portions of the amino acid sequence of Nivolumab, Pembrolizumab, Cemiplimab, Retifanlimab, Dostarlimab, Zimberclimab, Tiselelizumab, Camrelizumab, Sintilimab, Penpulimab, or any other anti-PD-1 antibody known in the art. In some embodiments, the nucleic acid sequence encoding the anti-PD-1 antibody codes for an amino acid sequence that comprises at least the CDRs of the variable heavy chain and the CDRs of the variable light chain portions of the amino acid sequence of Nivolumab, Pembrolizumab, Cemiplimab, Retifanlimab, Dostarlimab, Zimberclimab, Tiselelizumab, Camrelizumab, Sintilimab, Penpulimab, or any other anti-PD-1 antibody known in the art. The sequences of anti-PD-1 antibodies are described in the art and incorporated herein by reference as follows: Pembrolizumab (see U.S. Pat. Nos. 8,168,757, 8,354,509, 8,900,587, the contents of each of which is hereby incorporated by reference in its entirety), Cemiplimab (see U.S. Pat. No. 9,987,500 the contents of which is hereby incorporated by reference in its entirety), Retifanlimab (see US2019/0127467 the contents of which is hereby incorporated by reference in its entirety), Dostarlimab (see WO/2021/058711 the contents of which is hereby incorporated by reference in its entirety), Zimberelimab (see CN106432494 the contents of which is hereby incorporated by reference in its entirety), Tiselelizumab (see U.S. Pat. No. 8,735,553 the contents of which is hereby incorporated by reference in its entirety), Camrelizumab (see US2019/0309069 the contents of which is hereby incorporated by reference in its entirety), Sintilimab (see U.S. Pat. No. 10,316,089 the contents of which is hereby incorporated by reference in its entirety), Penpulimab (see US2019/0321466 the contents of which is hereby incorporated by reference in its entirety). The CDRs of certain anti-PD-1 antibodies are described in Jeong T J, Lee H T, Gu N, Jang Y J, Choi S B, Park U B, Lee S H, Heo Y S, The High-Resolution Structure Reveals Remarkable Similarity in PD-1 Binding of Cemiplimab and ostarlimab, the FDA-Approved Antibodies for Cancer Immunotherapy. Biomedicines, 2022 Dec. 6; 10 (12): 3154, the contents of which is hereby incorporated by reference in its entirety.

### Compositions

In some embodiments, a prophylactic or therapeutic composition of this disclosure comprises one or more antibodies (or one or more polynucleotides encoding one or more antibodies) and is administered in a pharmaceutical composition that includes a pharmaceutically acceptable carrier. In some embodiments, the prophylactic or therapeutic composition is comprised of one or more antibodies (or one or more polynucleotides encoding one or more antibodies) comprising SEQ ID NOs 5 and 11, SEQ ID NOs: 10 and 15, SEQ ID NOs: 16 and 21, SEQ ID NOs: 20 and 25, SEQ ID NOs: 27 and 28, or SEQ ID NOs: 27 and 33. In some embodiments, the pharmaceutical composition is in the form of a spray, aerosol, gel, solution, emulsion, nanoparticle (e.g., lipid nanoparticle), or suspension.

The composition is preferably administered to a subject with a pharmaceutically acceptable carrier. Typically, in some embodiments, an appropriate amount of a pharmaceutically acceptable salt is used in the formulation, which in some embodiments can render the formulation isotonic.

In certain embodiments, the one or more antibodies (or one or more polynucleotides encoding one or more antibodies) are provided as a composition comprising any one of the antibodies described herein (e.g., “anti-PAG” antibody) and a pharmaceutically acceptable carrier. In certain embodiments, the composition further comprises an adjuvant. In certain embodiments, the antibodies are conjugated with other molecules to increase their effectiveness as is known by those practiced in the art.

In some embodiments, the pharmaceutically acceptable carrier is selected from the group consisting of saline, Ringer's solution, dextrose solution, and a combination thereof. Other suitable pharmaceutically acceptable carriers known in the art are contemplated. Suitable carriers and their formulations are described in Remington's Pharmaceutical Sciences, 2005, Mack Publishing Co. The pH of the solution is preferably from about 5 to about 8, and more preferably from about 7 to about 7.5. The formulation may also comprise a lyophilized powder. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of antibodies being administered.

The phrase pharmaceutically acceptable carrier as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier is acceptable in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term carrier denotes an organic or inorganic ingredient, natural or synthetic, combined with the active ingredient to facilitate the application. The components of the pharmaceutical compositions also are capable of being comingled with the compounds of the present invention, and with each other, in a manner such that there is no interaction which would substantially impair the desired pharmaceutical efficiency. The composition may also include additional agents such as an isotonicity agent, a preservative, a surfactant, and, a divalent cation, preferably, zinc.

The composition can also include an excipient, or an agent for stabilization of an antibody composition, such as a buffer, a reducing agent, a bulk protein, amino acids (such as e.g., glycine or praline) or a carbohydrate. Typical carbohydrates useful in formulating compositions include but are not limited to sucrose, mannitol, lactose, trehalose, or glucose.

Surfactants may also be used to prevent soluble and insoluble aggregation and/or precipitation of antibodies included in the composition. Suitable surfactants include but are not limited to sorbitan trioleate, soya lecithin, and oleic acid. In certain cases, solution aerosols are preferred using solvents such as ethanol. Thus, formulations including antibodies can also include a surfactant that can reduce or prevent surface-induced aggregation of antibodies by atomization of the solution in forming an aerosol. Various conventional surfactants can be employed, such as polyoxyethylene fatty acid esters and alcohols, and polyoxyethylene sorbitol fatty acid esters. Amounts will generally range between 0.001% and 4% by weight of the formulation. In some embodiments, surfactants used with the present disclosure are polyoxyethylene sorbitan mono-oleate, polysorbate 80, polysorbate 20. Additional agents known in the art can also be included in the composition.

In some embodiments, the pharmaceutical compositions and dosage forms further comprise one or more compounds that reduce the rate by which an active ingredient will decay, or the composition will change in character. So called stabilizers or preservatives may include, but are not limited to, amino acids, antioxidants, pH buffers, or salt buffers. Nonlimiting examples of antioxidants include butylated hydroxy anisole (BHA), ascorbic acid and derivatives thereof, tocopherol and derivatives thereof, butylated hydroxy anisole and cysteine. Nonlimiting examples of preservatives include parabens, such as methyl or propyl p-hydroxybenzoate and benzalkonium chloride. Additional nonlimiting examples of amino acids include glycine or proline.

The present invention also teaches the stabilization (preventing or minimizing thermally or mechanically induced soluble or insoluble aggregation and/or precipitation of an inhibitor protein) of liquid solutions containing antibodies at neutral pH or less than neutral pH by the use of amino acids including proline or glycine, with or without divalent cations resulting in clear or nearly clear solutions that are stable at room temperature or preferred for pharmaceutical administration.

In one embodiment, the composition is a pharmaceutical composition of single unit or multiple unit dosage forms. Pharmaceutical compositions of single unit or multiple unit dosage forms of the invention comprise a prophylactically or therapeutically effective amount of one or more compositions (e.g., a compound of the invention, or other prophylactic or therapeutic agent), typically, one or more vehicles, carriers, or excipients, stabilizing agents, and/or preservatives. Preferably, the vehicles, carriers, excipients, stabilizing agents and preservatives are pharmaceutically acceptable.

In some embodiments, the pharmaceutical compositions and dosage forms comprise anhydrous pharmaceutical compositions and dosage forms. Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Suitable vehicles are well known to those skilled in the art of pharmacy, and non-limiting examples of suitable vehicles include glucose, sucrose, starch, lactose, gelatin, rice, silica gel, glycerol, talc, sodium chloride, dried skim milk, propylene glycol, water, sodium stearate, ethanol, and similar substances well known in the art. Saline solutions and aqueous dextrose and glycerol solutions can also be employed as liquid vehicles. Whether a particular vehicle is suitable for incorporation into a pharmaceutical composition or dosage form depends on a variety of factors well known in the art including, but not limited to, the way in which the dosage form will be administered to a patient and the specific active ingredients in the dosage form. Pharmaceutical vehicles can be sterile liquids, such as water and oils, including those of petroleum, animal, vegetable or synthetic origin, such as peanut oil, soybean oil, mineral oil, sesame oil and the like.

The invention also provides that a pharmaceutical composition can be packaged in a hermetically sealed container such as an ampoule or sachette indicating the quantity. In one embodiment, the pharmaceutical composition can be supplied as a dry sterilized lyophilized powder in a delivery device suitable for administration to the lower airways of a patient. If desired, the pharmaceutical compositions can be presented in a pack or dispenser device containing one or more unit dosage forms containing the active ingredient. The pack can for example comprise metal or plastic foil, such as a blister pack. Instructions for administration can accompany the pack or dispenser device.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for administration may be in the form of powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention (e.g., antibodies) as an active ingredient.

A liquid composition herein can be used as such with a delivery device, or they can be used for the preparation of pharmaceutically acceptable formulations comprising antibodies that are prepared for example by spray drying. The methods of spray freeze-drying proteins for pharmaceutical administration are disclosed in Maa et al., Curr. Pharm. Biotechnol., 2001, 1, 283-302, are incorporated herein. In another embodiment, the liquid solutions herein are freeze-spray-dried and the spray-dried product is collected as a dispersible peptide-containing powder that is therapeutically effective when administered to an individual.

The compounds and pharmaceutical compositions of the present invention can be employed in combination therapies, that is, the compounds and pharmaceutical compositions can be administered concurrently with, prior to, or subsequent to, one or more other desired therapeutics or medical procedures (e.g., antibodies can be used in combination treatment with another treatment such as antivirals or with a vaccine, and/or another treatment, including but not limited to an anti-PD-1 antibody). The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another therapeutic or prophylactic).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The current invention provides for dosage forms comprising peptides suitable for treating cancer or other diseases. The dosage forms can be formulated, e.g., as sprays, aerosols, nanoparticles, liposomes, or other forms known to one of skill in the art. See, e.g., Remington's Pharmaceutical Sciences; Remington: The Science and Practice of Pharmacy supra; Pharmaceutical Dosage Forms and Drug Delivery Systems by Howard C., Ansel et al., Lippincott Williams & Wilkins; 7^{th} edition (Oct. 1, 1999).

Generally, a dosage form used in the acute treatment of a disease may contain larger amounts of one or more of the active ingredients it comprises than a dosage form used in the chronic treatment of the same disease. In addition, the prophylactically and therapeutically effective dosage form may vary among different conditions. For example, a therapeutically effective dosage form may contain one or more antibodies that have an appropriate therapeutic action when intending to treat cancer or a viral infection such as HIV. On the other hand, a different effective dosage may contain one or more antibodies that have an appropriate prophylactic action when intending to prevent cancer or an infection caused by a virus (e.g, HIV). These and other ways in which specific dosage forms encompassed by this invention will vary from one another and will be readily apparent to those skilled in the art. See, e.g., Remington's Pharmaceutical Sciences, 2005, Mack Publishing Co.; Remington: The Science and Practice of Pharmacy by Gennaro, Lippincott Williams & Wilkins; 20^{th} edition (2003); Pharmaceutical Dosage Forms and Drug Delivery Systems by Howard C. Ansel et al., Lippincott Williams & Wilkins; 7^{th} edition (Oct. 1, 1999); and Encyclopedia of Pharmaceutical Technology, edited by Swarbrick, J. & J. C. Boylan, Marcel Dekker, Inc., New York, 1988, which are incorporated herein by reference in their entirety.

The pH of a pharmaceutical composition or dosage form may also be adjusted to improve delivery and/or stability of one or more active ingredients. Similarly, the polarity of a solvent carrier, its ionic strength, or tonicity can be adjusted to improve delivery. Compounds such as stearates can also be added to pharmaceutical compositions or dosage forms to alter advantageously the hydrophilicity or lipophilicity of one or more active ingredients to improve delivery. In this regard, stearates can also serve as a lipid vehicle for the formulation, as an emulsifying agent or surfactant, and as a delivery enhancing or penetration-enhancing agent. Different salts, hydrates, or solvates of the active ingredients can be used to adjust further the properties of the resulting composition.

Compositions can be formulated with appropriate carriers and adjuvants using techniques to yield compositions suitable for prophylaxis or treatment. The compositions can include an adjuvant, such as, for example but not limited to, alum, poly IC, MF-59, squalene-based adjuvants, or liposomal based adjuvants suitable for prophylaxis or treatment.

In some embodiments, the antibodies described herein are encoded by nucleic acids which are prepared in a mRNA-LNP or a DNA-LNP formulation for administration to a subject.

### Antibody Production

The antibodies disclosed herein can be produced by any method known in the art. In some embodiments, the antibodies disclosed herein are produced by culturing a cell transfected or transformed with a vector comprising nucleic acid sequences encoding an antibody described herein and isolating the antibody.

In some embodiments, antibodies are synthesized by the hybridoma culture method which results in antibodies that are not contaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques known in the art, including, for example, the hybridoma method (e.g., Kohler and Milstein., Nature, 256:495-97 (1975); Hongo et al, Hybridoma, 14 (3): 253-260 (1995), Harlow et al, Antibodies: A Laboratory Manual, (Cold Spring Harbor Laboratory Press, 2^{nd} cd. 1988); Hammerling et al, in: Monoclonal Antibodies and T-Cell Hybridomas 563-681 (Elsevier, N. Y., 1981)), recombinant DNA methods, phage-display technologies (see, e.g., Clackson et al, Nature, 352:624-628 (1991); Marks et al, J. Mol Biol. 222:581-597 (1992); Sidhu et al, J. Mol Biol. 338 (2): 299-310 (2004); Lee et al, J. Mol Biol. 340 (5): 1073-1093 (2004); Fellouse, Proc. Natl. Acad. ScL USA 101 (34): 12467-12472 (2004); and Lee et al, J. Immunol. Methods 284 (1-2): 119-132 (2004), and technologies for producing human or humanlike antibodies in animals that have parts or all of the human immunoglobulin loci or genes encoding human immunoglobulin sequences (see, e.g., Lonberg et al, Nature 368:856-859 (1994); Morrison, Nature 368:812-813 (1994); Fishwild et al, Nature Biotechnol 14:845-851 (1996); Neuberger, Nature Biotechnol. 14:826 (1996); and Lonberg and Huszar, Intern. Rev. Immunol. 13:65-93 (1995).

In some embodiments, expression of an antibody comprises expression vector(s) containing a polynucleotide that encodes an anti-PAG antibody. Methods that are well known to those skilled in the art can be used to construct expression vectors comprising antibody coding sequences and appropriate transcriptional and translational control signals. These methods include, for example, in vitro recombinant DNA techniques, synthetic techniques, and in vivo genetic recombination. Particular embodiments provide replicable vectors comprising a nucleotide sequence encoding an anti-PAG antibody disclosed herein operably linked to a promoter. In preferred embodiments, such vectors may include a nucleotide sequence encoding the heavy chain of an antibody molecule (or fragment thereof), a nucleotide sequence encoding the light chain of an antibody (or fragment thereof), or both the heavy and light chain.

The polynucleotide encoding the antibody may be modified, for example, by substituting the coding sequence for human heavy- and light-chain constant domains in place of the homologous murine sequences (U.S. Pat. No. 4,816,567; Morrison, et al, Proc. Natl Acad. ScL USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen. The monoclonal antibodies described herein may by monovalent, the preparation of which is well known in the art. For example, one method involves recombinant expression of an immunoglobulin light chain and a modified heavy chain. The heavy chain is generally truncated at any point in the Fc domain so as to prevent heavy chain crosslinking. Alternatively, the relevant cysteine residues may be substituted with another amino acid residue or are deleted so as to prevent crosslinking. In vitro methods are also suitable for preparing monovalent antibodies. Antibiotic ingestion to produce fragments, particularly Fab fragments, can be accomplished using routine techniques known in the art. Chimeric or hybrid antibodies also may be prepared in vitro using known methods in synthetic protein chemistry, including those involving crosslinking agents.

Various expression systems for producing antibodies are known in the art, and include, prokaryotic (e.g., bacteria), plant, insect, yeast, and mammalian expression systems. Suitable cell lines, can be transformed, transduced, or transfected with nucleic acids containing coding sequences for antibodies or portions of antibodies disclosed herein in order to produce the antibody of interest. Expression vectors containing such nucleic acid sequences, which can be linked to at least one regulatory sequence in a manner that allows expression of the nucleotide sequence in a host cell, can be introduced via methods known in the art. Practitioners in the art understand that designing an expression vector can depend on factors, such as the choice of host cell to be transfected and/or the type and/or amount of desired protein to be expressed. Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication. For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (e.g., antibodies and fragments thereof) is stably integrated into the genome of eukaryotic cells (for example mammalian cells), resulting in the stable expression of transfected genes. A gene that encodes a selectable marker (for example, resistance to antibiotics or drugs) can be introduced into host cells along with the gene of interest in order to identify and select clones that stably express a gene encoding a protein of interest. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired antibody molecule.

In some embodiments, the antibodies disclosed herein are encoded in a vector for expression in a cell line. In some embodiments, a vector comprises a polynucleotide sequence that encodes an anti-PAG antibody and the vector is transfected into one or more cell lines for expression. In some embodiments, one or more vectors comprise polynucleotide sequences encoding a light chain and a heavy chain of the antibody. For example, in some embodiments, a first vector may comprise a polynucleotide sequence encoding a light chain, a second vector may comprise a polynucleotide sequence encoding a heavy chain, of anti-PAG antibody. In some embodiments, both vectors are transfected into one or more cell lines for expression. A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of protein products may be important for the function of the antibody. Different host cell strains have characteristic and specific mechanisms for the post-translational processing and modification of proteins and gene products. As such, appropriate host systems or cell lines can be chosen to ensure the correct modification and processing of the foreign antibody expressed. Thus, eukaryotic host cells possessing the cellular machinery for proper processing of the primary transcript, glycosylation, and phosphorylation of the gene product may be used.

CAR T cells that co-express the CAR and immune modulating antibodies, including anti-PD-1, have remarkable anti-tumor capacity. Persistent T cell activation can lead to severe immune-related adverse events and cytokine release syndrome (CRS). By limiting the expression of anti-PD-1 antibody to IFN-γ secreting CAR T cells, the cells that expressed the antibody exhibited lower PD-1 expression, increased T cell activation, and greater anti-tumor activity against PD-L1 positive tumor cell lines. See Mortazavi A, Williams B A, McCuc K, Schaeffer L, Wold B. Mapping and quantifying mammalian transcriptomes by RNA-Seq. Nature methods. July 2008; 5 (7): 621-628. Likewise, in some embodiments, the anti-PAG antibodies described herein can be expressed in IFN-γ secreting chimeric antigen receptor (CAR) T cells.

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2^{nd} Ed., Rickwood, D. and Hames, B. D., eds. (Oxford University Press: New York, 1992)). Cell culturing conditions can vary according to the type of host cell selected. Commercially available media can be utilized.

Antibodies disclosed herein can be purified from any human or non-human cell that expresses the antibody, including those that have been transfected with expression constructs that express the antibody or fragments thereof. For antibody recovery, isolation and/or purification, the cell culture medium or cell lysate is centrifuged to remove particulate cells and cell debris. The desired antibody molecule is isolated or purified away from contaminating soluble proteins and polypeptides by suitable purification techniques. Non-limiting purification methods for proteins/antibodies include: size exclusion chromatography, affinity chromatography, ion exchange chromatography, ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

### Methods of Treatment

In one embodiment, the subject matter disclosed herein relates to a preventive medical treatment started after following diagnosis of a disease (e.g., cancer) in order to prevent the disease from worsening or curing the disease. In one embodiment, the subject matter disclosed herein relates to prophylaxis of subjects who are believed to be at risk for moderate or severe disease associated with cancer or have previously been diagnosed with another disease, such as cancer. In one embodiment, the subjects can be administered the pharmaceutical composition described herein. The invention contemplates using any of the antibodies produced by the systems and methods described herein. In one embodiment, the compositions described herein can be administered subcutaneously via syringe or any other suitable method know in the art.

The compound(s) or combination of compounds (e.g., anti-PAG antibody in combination with anti-PD-1 antibody) disclosed herein, or pharmaceutical compositions may be administered to a cell, mammal, or human by any suitable means. Non-limiting examples of methods of administration include, among others, (a) administration though oral pathways, which includes administration in capsule, tablet, granule, spray, syrup, or other such forms; (b) administration through non-oral pathways such as intraocular, intranasal, intraauricular, rectal, vaginal, intraurethral, transmucosal, buccal, or transdermal, which includes administration as an aqueous suspension, an oily preparation or the like or as a drip, spray, suppository, salve, ointment or the like; (c) administration via injection, including subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; (e) administration topically; as deemed appropriate by those of skill in the art for bringing the compound or combination of compounds disclosed herein into contact with living tissue; (f) administration via inhalation, including through aerosolized, nebulized, and powdered formulations; (g) administration through implantation; and administration via electroporation.

In some embodiments, one or more antibodies disclosed herein are prepared in a cocktail of DNA-encoding antibodies or mRNA-encoding antibodies and delivered by electroporation to a subject for in vivo expression of the encoded antibodies.

As will be readily apparent to one skilled in the art, the effective in vivo dose to be administered and the particular mode of administration will vary depending upon the age, weight and species treated, and the specific use for which the compound or combination of compounds disclosed herein are employed. The determination of effective dose levels, that is the dose levels necessary to achieve the desired result, can be accomplished by one skilled in the art using routine pharmacological methods. Typically, human clinical applications of products are commenced at lower dose levels, with dose level being increased until the desired effect is achieved. Alternatively, acceptable in vitro studies can be used to establish useful doses and routes of administration of the compositions identified by the present methods using established pharmacological methods. Effective animal doses from in vivo studies can be converted to appropriate human doses using conversion methods known in the art (e.g., see Nair A B, Jacob S. A simple practice guide for dose conversion between animals and human. Journal of basic and clinical pharmacy. 2016 March; 7(2):27.)

### Methods of Prevention

In some embodiments, the compositions prepared using methods of the invention can be used as a vaccine to promote an immune response against future disease (e.g., cancer). In some embodiments, the antibodies are neutralizing antibodies.

In some embodiments, the antibodies (or polynucleotides encoding antibodies) prepared using methods of the invention can be combined with additional pharmaceutical components.

### Dosage

A prophylactically effective or therapeutically effective amount is typically dependent on the subject's weight, the subject's physical condition, the extensiveness of the condition to be treated, and the subject's age. In general, an anti-PAG antibody, or polynucleotides encoding one or more antibodies, disclosed herein may be administered in an amount in the range of about 10 ng/kg body weight to about 100 mg/kg body weight per dose. In some embodiments, antibodies may be administered in an amount in the range of about 50 μg/kg body weight to about 5 mg/kg body weight per dose. In some embodiments, antibodies may be administered in an amount in the range of about 100 μg/kg body weight to about 10 mg/kg body weight per dose. In some embodiments, antibodies may be administered in an amount in the range of about 100 μg/kg body weight to about 20 mg/kg body weight per dose. In some embodiments, antibodies may be administered in an amount in the range of about 0.5 mg/kg body weight to about 20 mg/kg body weight per dose. In some embodiments, antibodies may be administered in an amount in the range of about 0.5 mg/kg body weight to about 10 mg/kg body weight per dose. In some embodiments, antibodies may be administered in an amount in the range of about 1 mg/kg body weight to about 5 mg/kg body weight per dose. In some embodiments, antibodies may be administered in an amount in the range of about 0.1 mg/kg body weight to about 0.5 mg/kg body weight per dose. In some embodiments, antibodies may be administered in a dose of at least about 100 μg/kg body weight, at least about 250 μg/kg body weight, at least about 500 μg/kg body weight, at least about 750 μg/kg body weight, at least about 3 mg/kg body weight, at least about 5 mg/kg body weight, or at least about 10 mg/kg body weight.

In some methods, the dosage is adjusted to achieve a plasma antibody concentration of about 1-1000 μg/mL or about 25-300 μg/mL. In some embodiments, the dosage is adjusted to achieve a plasma antibody concentration of about 0.001 μg/mL to about 10 μg/mL. In some embodiments, the dosage is adjusted to achieve a plasma antibody concentration of about 1 μg/mL to about 10 μg/mL. In some embodiments, the dosage is adjusted to achieve a plasma antibody concentration of about 0.01 μg/mL to about 1 μg/mL. In some embodiments, the dosage is adjusted to achieve a plasma antibody concentration of about 0.01 μg/mL to about 0.1 μg/mL.

### Kits of the Invention

In one embodiment, the subject matter disclosed herein relates to a kit for generating an anti-PAG antibody comprising an antibody composition of the present invention and instructions for use. In one embodiment, the subject matter disclosed herein relates to a kit for generating an anti-PAG antibody comprising one or more vectors comprising a polynucleotide sequence of any of the an anti-PAG antibodies described above targeting PAG. The kit can further include at least one additional reagent or one or more antibodies of the present invention. The kit usually has a label indicating the intended use of the kit contents. The term label includes all documents and is attached to the kit or with the kit, or otherwise attached to the kit.

### Non-Limiting Embodiments of the Subject Matter

In certain aspects, the present disclosure provides a monoclonal antibody or a fragment thereof, comprising: a first arm comprising a first variable heavy chain domain and a first variable light chain domain, wherein a portion of the first arm is capable of binding to an extracellular portion of human PAG; and a second arm comprising a second variable heavy chain domain and a second variable light chain domain, wherein a portion of the second arm is capable of binding to an extracellular portion of human PAG. In some embodiments, the first and second arms each further comprise a fragment, crystallizable (Fc) domain.

In some embodiments, the first and second arms of the monoclonal antibody each further comprise a CH1 domain, a hinge domain, and a CL domain.

In some embodiments, the first variable heavy chain domain of the first arm of the monoclonal antibody is encoded by a first polypeptide chain. In some embodiments, the first variable light chain domain of the first arm of the monoclonal antibody is encoded by a second polypeptide chain. In some embodiments, the second variable heavy chain domain of the second arm of the monoclonal antibody is encoded by a third polypeptide chain. In some embodiments, the second variable light chain domain of the second arm of the monoclonal antibody is encoded by a fourth polypeptide chain. In some embodiments, the first variable heavy chain domain and first variable light chain domain of the monoclonal antibody form a first PAG binding site. In some embodiments, the second variable heavy chain domain and second variable light chain domain of the monoclonal antibody form a second PAG binding site. In some embodiments, the first and second PAG binding sites are the same. In some embodiments, the first and third polypeptide chain each further encode a hinge domain, a CH1 domain, and the Fc domain, and wherein the second and fourth polypeptide chain each further encode a CL domain. In some embodiments, the first and third polypeptide chains comprise the same sequence and the second and fourth polypeptide chains comprise the same sequence. In some embodiments, the first and third polypeptide chain each comprises an amino acid sequence comprising SEQ ID NO: 5, 10, 16, 20, or 27 and the second and fourth polypeptide chain each comprises an amino acid sequence comprising SEQ ID NO: 11, 15, 21, 25, 28, or 33.

In some embodiments, the first variable heavy chain domain comprises an amino acid sequence comprising SEQ ID NO: 5, 16, or 27, and the first variable light chain domain comprises an amino acid sequence comprising SEQ ID NO: 11, 21, 28, or 33.

In some embodiments, the first arm comprises an amino acid sequence comprising SEQ ID NO: 5 and the second arm comprises an amino acid sequence comprising SEQ ID NO: 11, the first arm comprises an amino acid sequence comprising SEQ ID NO: 10 and the second arm comprises an amino acid sequence comprising SEQ ID NO: 15, the first arm comprises an amino acid sequence comprising SEQ ID NO: 16 and the second arm comprises an amino acid sequence comprising SEQ ID NO: 21, the first arm comprises an amino acid sequence comprising SEQ ID NO: 20 and the second arm comprises an amino acid sequence comprising SEQ ID NO: 25, the first arm comprises an amino acid sequence comprising SEQ ID NO: 27 and the second arm comprises an amino acid sequence comprising SEQ ID NO: 28, or the first arm comprises an amino acid sequence comprising SEQ ID NO: 27 and the second arm comprises an amino acid sequence comprising SEQ ID NO: 33.

In some embodiments, the first and second variable heavy chain domains each comprises a CDR-H1, CDR-H2, and CDR-H3 domain, and the first and second variable light chain domains each comprises a CDR-L1, CDR-L2, and CDR-L3 domain. In some embodiments, the CDR-H1, CDR-H2, and CDR-H3 of the first heavy chain domains and the CDR-H1, CDR-H2, and CDR-H3 of the second heavy chain domains comprises an amino acid sequence comprising SEQ ID NO: 6, 7, and 8, respectively, and wherein the CDR-L1, CDR-L2, and CDR-L3 of the first light chain domains and the CDR-L1, CDR-L2, and CDR-L3 of the second light chain domains comprises an amino acid sequence comprising SEQ ID NO: 12, 13, and 14, respectively.

In some embodiments, the first and second polypeptide chains are linked by one or more covalent disulfide bonds and the third and fourth polypeptide chains are linked by one or more covalent disulfide bonds. In some embodiments, the first and third polypeptide chains are linked by one or more covalent disulfide bonds.

In certain aspects, the present disclosure provides a scFv comprising a polypeptide comprising a variable heavy chain domain and a variable light chain domain. In some embodiments, the variable heavy chain domain and variable light chain domain form a binding site to an extracellular portion of human PAG.

In some embodiments, the scFv further comprises a linker between the variable heavy chain domain and a variable light chain domain. In some embodiments, the linker is a glycine serine linker. In some embodiments, the linker is a glycine serine linker about 15 amino acids in length. In some embodiments, the linker comprises SEQ ID NO: 26. In some embodiments, the polypeptide further comprises an Fc domain. In some embodiments, the variable heavy chain domain comprises an amino acid sequence comprising SEQ ID NO: 5, 16, or 27, and the variable light chain domain comprises an amino acid sequence comprising SEQ ID NO: 11, 21, 28, or 33. In some embodiments, the variable heavy chain domain comprises an amino acid sequence comprising SEQ ID NO: 5 and the variable light chain domain comprises an amino acid sequence comprising SEQ ID NO: 11, the variable heavy chain domain comprises an amino acid sequence comprising SEQ ID NO: 16 and the variable light chain domain comprises an amino acid sequence comprising SEQ ID NO: 21, the variable heavy chain domain comprises an amino acid sequence comprising SEQ ID NO: 27 and the variable light chain domain comprises an amino acid sequence comprising SEQ ID NO: 28, or the variable heavy chain domain comprises an amino acid sequence comprising SEQ ID NO: 27 and the variable light chain domain comprises an amino acid sequence comprising SEQ ID NO: 33.

In some embodiments, the variable heavy chain domain comprises a CDR-H1, CDR-H2, and CDR-H3 domain, and the variable light chain domain comprises a CDR-L1, CDR-L2, and CDR-L3 domain. In some embodiments, the CDR-H1, CDR-H2, and CDR-H3 of the heavy chain domain comprises an amino acid sequence comprising SEQ ID NO: 6, 7, and 8, respectively, and wherein the CDR-L1, CDR-L2, and CDR-L3 of the light chain domain comprises an amino acid sequence comprising SEQ ID NO: 12, 13, and 14, respectively.

In some embodiments, the polypeptide further comprises a second variable heavy chain domain and a second variable light chain domain. In some embodiments, the second variable heavy chain domain and second variable light chain domain form a second binding site to an extracellular portion of human PAG. In some embodiments, the scFv further comprises a linker between the second variable heavy chain domain and the second variable light chain domain. In some embodiments, the linker is a glycine serine linker. In some embodiments, the linker is a glycine serine linker about 15 amino acids in length. In some embodiments, the linker comprises SEQ ID NO: 26. In some embodiments, the first and second PAG binding sites are the same. In some embodiments, the first and second variable heavy chain domains comprise the same sequence and the first and second variable light chain domains comprise the same sequence. In some embodiments, the first and second variable heavy chain domains each comprises an amino acid sequence comprising SEQ ID NO: 5, 16, or 27, and wherein the first and second variable light chain domains each comprise an amino acid sequence comprising SEQ ID NO: 11, 21, 28, or 33. In some embodiments, the first and second variable heavy chain domains each comprise an amino acid sequence comprising SEQ ID NO: 5 and the first and second variable light chain domains each comprise an amino acid sequence comprising SEQ ID NO: 11, the first and second variable heavy chain domains each comprise an amino acid sequence comprising SEQ ID NO: 16 and the first and second variable light chain domains each comprise an amino acid sequence comprising SEQ ID NO: 21, the first and second variable heavy chain domains each comprise an amino acid sequence comprising SEQ ID NO: 27 and the first and second variable light chain domains each comprise an amino acid sequence comprising SEQ ID NO: 28, or the first and second variable heavy chain domains each comprise an amino acid sequence comprising SEQ ID NO: 27 and the first and second variable light chain domains each comprise an amino acid sequence comprising SEQ ID NO: 33.

In some embodiments, the first and second variable heavy chain domains each comprises a CDR-H1, CDR-H2, and CDR-H3 domain, and the first and second variable light chain domains each comprises a CDR-L1, CDR-L2, and CDR-L3 domain. In some embodiments, the CDR-H1, CDR-H2, and CDR-H3 of the first heavy chain domains and the CDR-H1, CDR-H2, and CDR-H3 of the second heavy chain domains comprises an amino acid sequence comprising SEQ ID NO: 6, 7, and 8, respectively. In some embodiments, the CDR-L1, CDR-L2, and CDR-L3 of the first light chain domains and the CDR-L1, CDR-L2, and CDR-L3 of the second light chain domains comprises an amino acid sequence comprising SEQ ID NO: 12, 13, and 14, respectively.

In some embodiments, the Fc domain of the polypeptide associates with the Fc domain of a second polypeptide, wherein the second polypeptide is identical to the first polypeptide. In some embodiments, the Fc region of the first polypeptide comprises knob mutations and the Fc region of the second polypeptide comprise hole mutations, or vice versa.

In some embodiments, the monoclonal antibody or scFv is capable of localizing a PAG protein away from an immune synapse.

In some embodiments, the monoclonal antibody or scFv is capable of localizing a PD-1 protein away from an immune synapse.

In some embodiments, the monoclonal antibody or scFv is capable of disrupting downstream signaling of a PD-1 mediated response in a T cell.

In some embodiments, the monoclonal antibody or scFv is capable of disrupting downstream signaling of a PAG mediated response in a T cell.

In some embodiments, the monoclonal antibody or scFv is capable of enhancing T cell function.

In some embodiments, the PAG protein is located on a T cell, and the monoclonal antibody or scFv is capable of preventing the phosphorylation of PAG protein downstream of PD-1 signaling.

In some embodiments, the monoclonal antibody or scFv is capable of inducing a cytokine secretion in a T cell. In some embodiments, the cytokine secretion is a secretion of IL-2.

In certain aspects, the present disclosure provides a pharmaceutical composition comprising: the monoclonal antibody or the scFv described herein; and a pharmaceutically acceptable carrier.

In certain aspects, the present disclosure provides a method of preventing or treating cancer in a subject comprising administering to the subject an effective amount of any of the pharmaceutical compositions described herein, for example, a pharmaceutical composition comprising: the monoclonal antibody or the scFv described herein; and a pharmaceutically acceptable carrier.

In some embodiments, the cancer is selected from colorectal cancer, lung cancer, bladder cancer, breast cancer, cervical cancer, kidney cancer, leukemia, Hodgkin lymphoma, non-Hodgkin lymphoma, prostate cancer, skin cancer (e.g., melanoma), head and neck cancer, endometrial cancer, colon cancer, rectal cancer, liver cancer, thyroids cancer, esophageal cancer, renal cell cancer, testicular cancers, and a combination thereof.

In some embodiments, the cancer is selected from colorectal cancer, colon adenocarcinoma, renal cell carcinoma, melanoma, acute myeloid leukemia, invasive breast cancer, cervical squamous cancer, and testicular cancer. In some embodiments, the cancer is colorectal cancer. In some embodiments, the cancer is melanoma.

In some embodiments, the administration of the pharmaceutical composition is capable of inhibiting tumor growth. In some embodiments, the administration of the pharmaceutical composition is capable of increasing T cell infiltration in the tumor.

In some embodiments, the pharmaceutical composition is administered in combination with an immune checkpoint therapy. In some embodiments, the immune checkpoint therapy is an anti-PD-1 antibody. In some embodiments, the administration of the pharmaceutical composition in combination with the anti-PD-1 antibody enhances the anti-PD-1 response.

In certain aspects, the present disclosure provides a kit for generating a monoclonal antibody or fragment thereof or an scFv, the kit comprising one or more vectors comprising a polynucleotide sequence encoding any of the monoclonal antibodies or any of the scFvs described herein.

In certain aspects, the present disclosure provides a kit for generating a monoclonal antibody or fragment thereof, the kit comprising: a first vector comprising a polynucleotide sequence encoding the first polypeptide chain of any of the monoclonal antibody described herein; and a second vector comprising a polynucleotide sequence encoding the second polypeptide chain of any of the monoclonal antibody described herein.

In some embodiments, the first vector and the second vector are the same vector. In some embodiments, the first vector and the second vector are two different vectors.

In certain aspects, the present disclosure provides one or more host cells comprising: one or more vectors comprising a polynucleotide sequence encoding any of the monoclonal antibodies or any of the scFvs described herein.

In certain aspects, the present disclosure provides one or more host cells comprising: a first vector comprising a polynucleotide sequence encoding the first polypeptide chain of any of the monoclonal antibody described above; and a second vector comprising a polynucleotide sequence encoding the second polypeptide chain of any of the monoclonal antibody described above.

In some embodiments, the first vector and the second vector are the same vector. In some embodiments, the first vector and the second vector are two different vectors.

In certain aspects, the present disclosure provides a method of making a monoclonal antibody or fragment thereof or scFv comprising: culturing the one or more host cells described herein under conditions suitable for an expression of the one or more vectors; and recovering the monoclonal antibody or fragment thereof or scFv.

In certain aspects, the present disclosure provides a method of making a monoclonal antibody or fragment thereof comprising: culturing the one or more host cells described herein under conditions suitable for an expression of the first vector and the second vector; and recovering the monoclonal antibody or fragment thereof.

In certain aspects, the present disclosure provides a composition comprising: one or more vectors comprising a polynucleotide sequence encoding any of the monoclonal antibodies or any of the scFvs described above.

In certain aspects, the present disclosure provides a composition comprising: a first vector comprising a polynucleotide sequence encoding the first polypeptide chain of any of the monoclonal antibody described herein; and a second vector comprising a polynucleotide sequence encoding the second polypeptide chain of any of the monoclonal antibody. In some embodiments, the first vector and the second vector are the same vector. In some embodiments, the first vector and the second vector are two different vectors.

In certain aspects, the present disclosure provides a means for binding an extracellular portion of a human PAG protein. In some embodiments, the means for binding an extracellular portion of a human PAG protein comprises: a first arm comprising a first variable heavy chain domain and a first variable light chain domain, wherein a portion of the first arm is capable of binding to the extracellular portion of the human PAG protein; and a second arm comprising a second variable heavy chain domain and a second variable light chain domain, wherein a portion of the second arm is capable of binding to the extracellular portion of the human PAG protein, wherein the first and second arms each further comprise a fragment, crystallizable (Fc) domain. In some embodiments, the first and second arms each further comprise a CH1 domain, a hinge domain, and a CL domain.

In some embodiments, the first variable heavy chain domain of the first arm is encoded by a first polypeptide chain; the first variable light chain domain of the first arm is encoded by a second polypeptide chain; the second variable heavy chain domain of the second arm is encoded by a third polypeptide chain; the second variable light chain domain of the second arm is encoded by a fourth polypeptide chain; and the first variable heavy chain domain and first variable light chain domain form a first PAG binding site and the second variable heavy chain domain and second variable light chain domain form a second PAG binding site. In some embodiments, the first and second PAG binding sites are the same. In some embodiments, the first and third polypeptide chain each further encode a hinge domain, a CH1 domain, and the Fc domain, and the second and fourth polypeptide chain each further encode a CL domain. In some embodiments, the first and third polypeptide chains comprise the same sequence and the second and fourth polypeptide chains comprise the same sequence.

In some embodiments, the first variable heavy chain domain comprises an amino acid sequence of the variable heavy chain portion of SEQ ID NO: 5, 16, or 27, wherein the first variable light chain domain comprises an amino acid sequence of the variable light chain portion of SEQ ID NO: 11, 21, 28, or 33.

In some embodiments, the first arm comprises an amino acid sequence comprising SEQ ID NO: 5 and the second arm comprises an amino acid sequence comprising SEQ ID NO: 11, the first arm comprises an amino acid sequence comprising SEQ ID NO: 10 and the second arm comprises an amino acid sequence comprising SEQ ID NO: 15, the first arm comprises an amino acid sequence comprising SEQ ID NO: 16 and the second arm comprises an amino acid sequence comprising SEQ ID NO: 21, the first arm comprises an amino acid sequence comprising SEQ ID NO: 20 and the second arm comprises an amino acid sequence comprising SEQ ID NO: 25, the first arm comprises an amino acid sequence comprising SEQ ID NO: 27 and the second arm comprises an amino acid sequence comprising SEQ ID NO: 28, or the first arm comprises an amino acid sequence comprising SEQ ID NO: 27 and the second arm comprises an amino acid sequence comprising SEQ ID NO: 33.

In some embodiments, the means comprises any one of the scFvs described herein.

In some embodiments, the means is capable of localizing a PD-1 protein away from an immune synapse. In some embodiments, the means is capable of localizing a PAG protein away from an immune synapse.

In some embodiments, the means is capable of disrupting downstream signaling of a PD-1 mediated response in a T cell.

In some embodiments, the means is capable of disrupting downstream signaling of a PAG mediated response in a T cell. In some embodiments, the means is capable of enhancing T cell function.

In some embodiments, the PAG protein is located on a T cell, and the means is capable of preventing the phosphorylation of PAG protein downstream of PD-1 signaling. In some embodiments, the means is capable of inducing a cytokine secretion in a T cell. In some embodiments, the cytokine secretion is a secretion of IL-2.

In some embodiments, in the methods disclosed herein the subject is a human subject.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Neutralization of the Adaptor Protein PAG by Monoclonal Antibody Limits Murine Tumor Growth

Immune checkpoint therapy is a relatively new modality in the treatment of cancer. Specifically, PD-1 and PD-L1 targeting antibodies release the breaks on a patient's T cells, allowing a more robust anti-tumor immune response. It is well established that enhanced T cell infiltration into tumors and activation correlate with better prognosis. It is through increased T cell activation that PD-1 blockade leads to tumor identification and subsequent clearance. Despite great promise for success, the average response rate to PD-1 blockade for most tumors is 23%, (see Refs. 1-3), leaving open the opportunity for improvement.

It was recently shown that phosphoprotein associated with glycosphingolipid-enriched microdomains 1 (PAG), a member of the transmembrane adaptor protein (TRAP) family, is phosphorylated after PD-1 is ligated by PD-L1 or PD-L2, and that PAG phosphorylation is associated with inhibition of various T cell functions downstream of PD-1. See Ref. 3. Furthermore, it was shown that two murine tumors, colon adenocarcinoma MC38 and melanoma B16, exhibited limited growth in PAG knockout (KO) mice, with enhanced sensitivity to PD-1 blockade. See Ref. 3. Through T cell adoptive transfer experiments of PAG KO T cells into wild-type, tumor-bearing mice, it was shown that this function of PAG in the context of the tumor immune response is T cell intrinsic. See Ref. 3.

Through confocal live imaging, it was also established that PAG localizes to the point of contact or immune synapse between a T cell and antigen-presenting cell (APC), and that this localization is essential for its function in the PD-1 pathway. See Ref. 3. Accordingly, described herein is testing of whether targeting PAG in vivo through antibody binding could neutralize its inhibitory function. In this way, combined antibody administration of anti-PAG and anti-PD-1 antibodies to cancer patients can enhance the anti-tumor immune response and overall patient survival. To test this hypothesis, antibodies targeting human PAG in mice were generated and the efficacy of these antibodies assayed with respect to binding and neutralizing PAG function in vivo.

### The Extracellular Portion of PAG is a Feasible Antibody Target

PAG has a short, 16 amino acid extracellular portion or ectodomain that is targeted through the disclosed immunization strategy. Notably, PAG does not have a signaling peptide that aides in its localization at the plasma membrane leaving open the possibility that the extracellular domain of PAG may be cleaved during protein processing. To first demonstrate that the 16 amino acid extracellular domain is intact on full-length PAG, PAG-GFP was stably expressed in A549 lung epithelial cells (that do not express endogenous PAG), enriched for the protein in the cell lysate through immunoprecipitation with anti-GFP-antibody conjugated beads, and mass spectrometry was performed. The extracellular domain of PAG is not cleaved in protein processing and is present on full-length PAG (FIG. 1A). De novo peptide structure prediction (see Ref. 4) was then used on the extracellular portion of human and mouse PAG to demonstrate that it has a tertiary structure (FIG. 1B). Notably, the predicted structures of human and mouse PAG extracellular portions are highly similar and have a low hydropathy index, indicative of low hydrophobicity. See Ref. 5. To support the role of PAG in activated T-cells It was shown that upon stimulation with anti-CD3 and anti-CD28 antibodies, PAG expression is increased and less phosphorylated (FIG. 1C). See Ref. 6.

As previously stated, PAG functions downstream of PD-1 in T cells following stimulation. See Ref. 3. Additionally, when a T-cell and APC interact, the immune synapse is formed between the two cells. PAG and PD-1 both polarize to the immune synapse. See Ref. 3. To determine if PAG and PD-1 are co-localized within close proximity following PD-1 ligation, the proximity ligation assay (PLA) was utilized in combination with the PD-L2 overexpressing Raji B cell-Jurkat T-cell co-culture system in the presence of SEE. PLA utilizes primary antibodies from different host species directed against two endogenously expressed proteins followed by secondary antibodies termed PLA probes which are tagged with a short sequence-specific DNA tag. If the two proteins are within a proximity of 40 nm or less, the PLA probes are close enough to interact and the DNA strands can participate in rolling circle DNA synthesis. Fluorescent-labeled complementary oligonucleotide probes then bind the resulting amplified DNA circle, and these fluorescent clusters are indicative of proteins within proximity when viewed by fluorescent microscopy. Endogenous PAG and PD-1 are shown to be within 40 nm of each other following PD-1 ligation when PAG and PD-1 polarize to the immune synapse (FIG. 1D).

To test the exclusion of PAG from the immune synapse to disrupt co-localization with PD-1, full-length PAG-GFP was expressed or full-length PAG conjugated to human IgG Fc (200 amino acids) at its N terminus and expressed extracellularly (Fc-PAG-GFP) in Jurkat T-cells, to mimic antibody binding and the potential resulting steric hindrance (FIG. 1E). These transfected Jurkat T-cells were then co-cultured with Raji B cells and imaged the cells for PAG localization. PAG-GFP was more enriched at the contact site than Fc-PAG-GFP (FIGS. 1F and 1G). Further, Fc-PAG-GFP was excluded from the immune synapse more often than PAG-GFP (FIGS. 1F and 1G). To correlate PAG exclusion from the synapse to T cell function, IL-2 secretion from T cell-Raji cell conjugates that express either PAG-GFP, or Fc-PAG-GFP was measured and revealed impaired ability of PD-1 to inhibit cytokine secretion (FIG. 1H). These observations suggest that antibody bound to PAG could lead to mislocalization and PAG dysfunction in the immune synapse context.

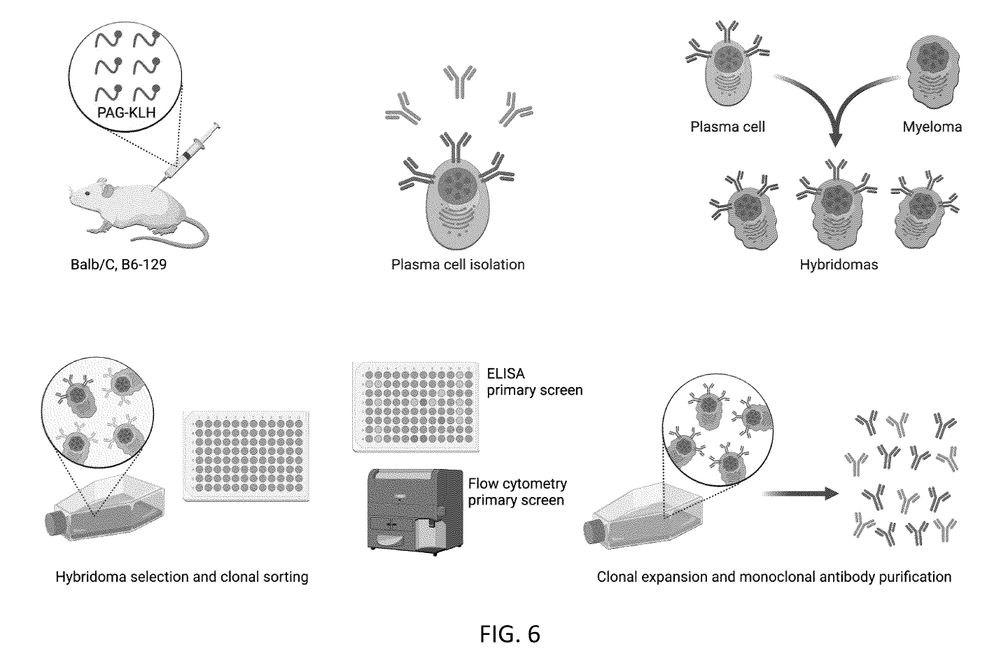

To develop antibodies to the extracellular portion of PAG, Balb/C and B6-129 mice were immunized with amino acids 1-16 of human PAG in combination with keyhole limpet haemocyanin (KLH). This large, copper-containing protein is a highly immunogenic T-cell antigen. Two boosters of bovine serum albumin (BSA)-PAG were administered. ELISA did the primary serum screen. Hybridoma libraries were made by fusing myeloma cells with B cells derived from the spleens of positive serum mice. The resulting immortalized B cells (hybridomas) produce monoclonal antibodies. Hybridoma supernatants were first screened by ELISA, and then by flow cytometry binding to intact cells (FIG. 6).

### Pag Antibody Clone 7M16A Binds to Human Pag

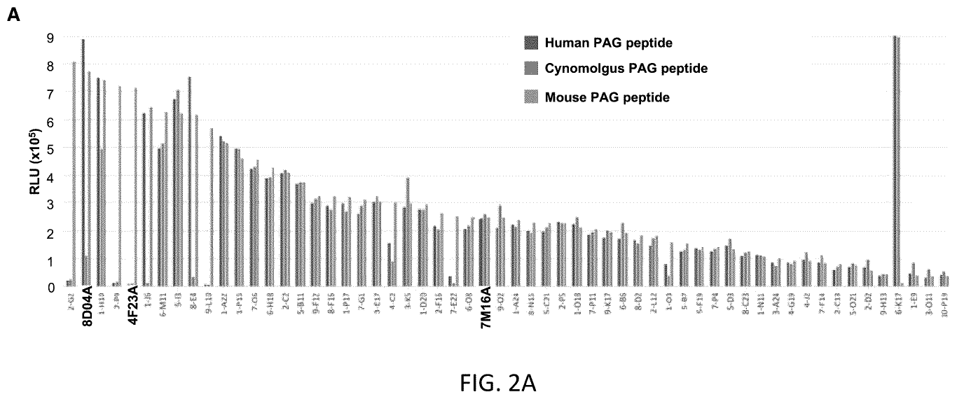

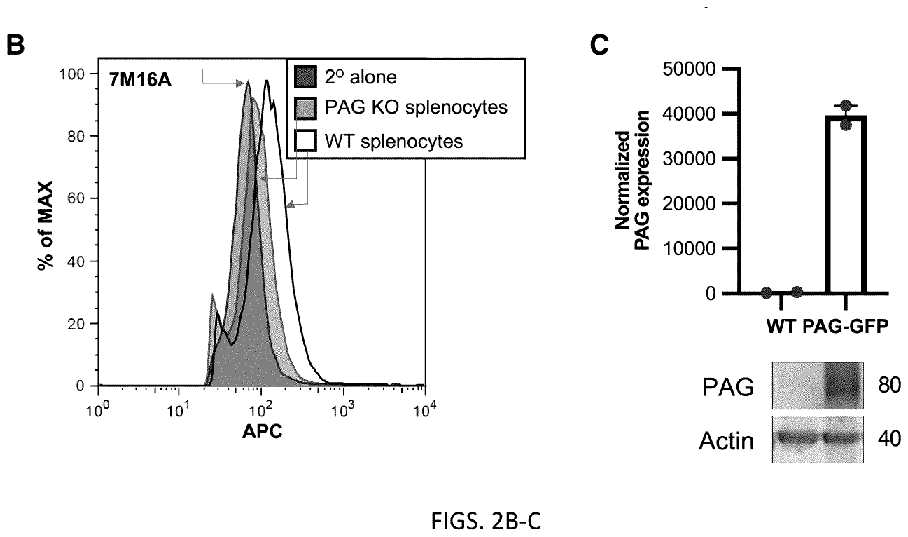

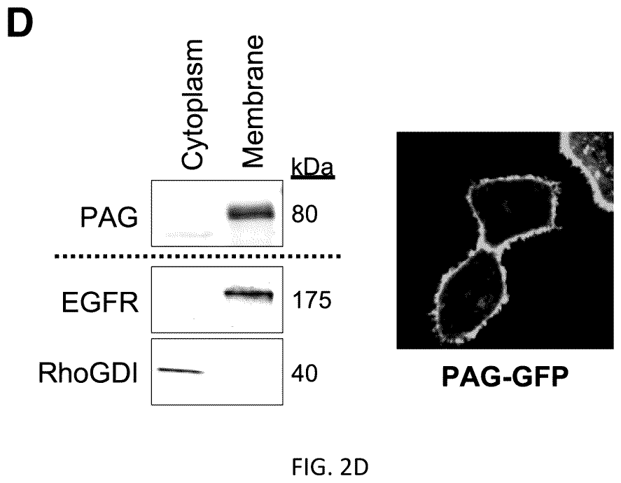

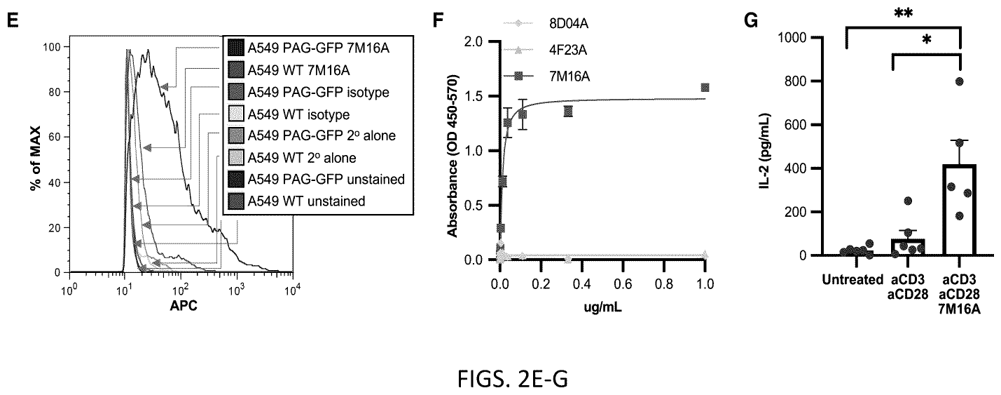

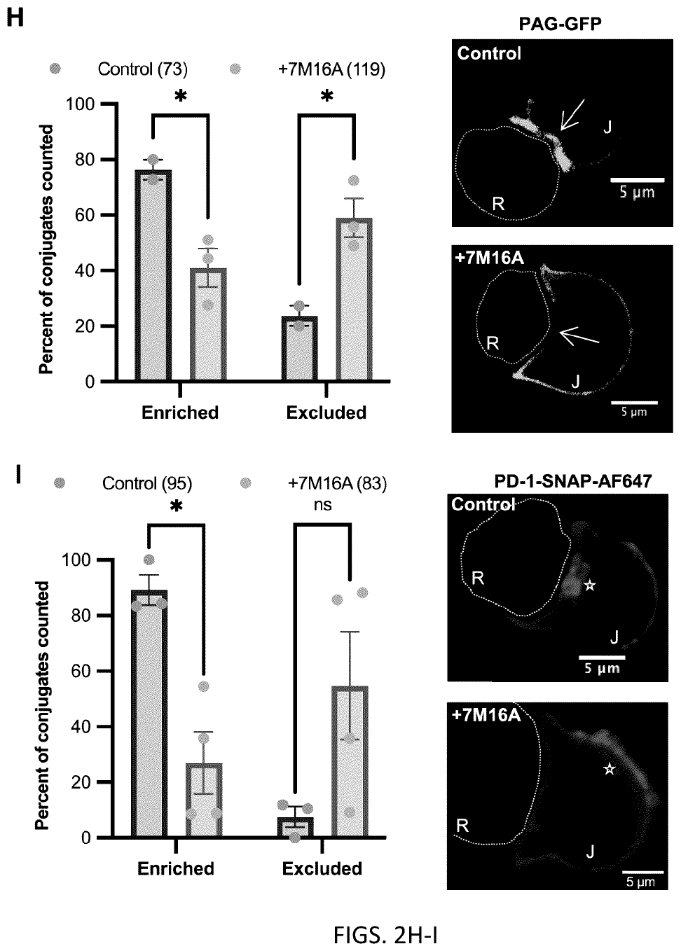

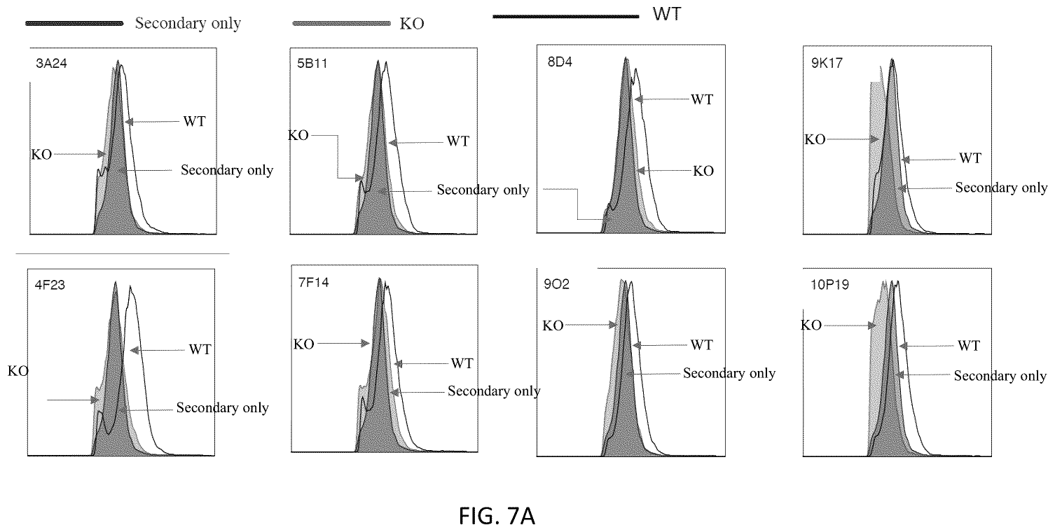

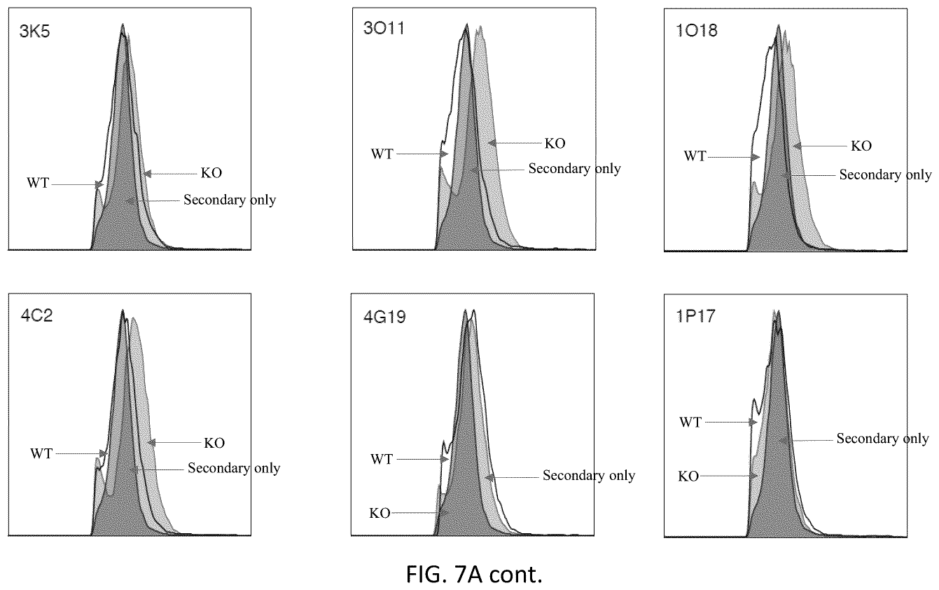

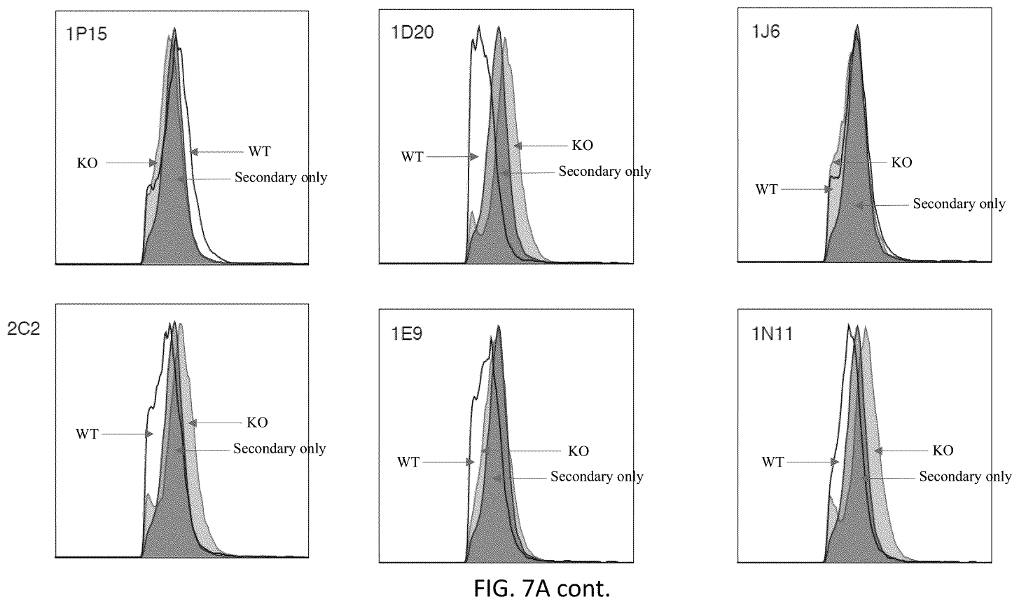

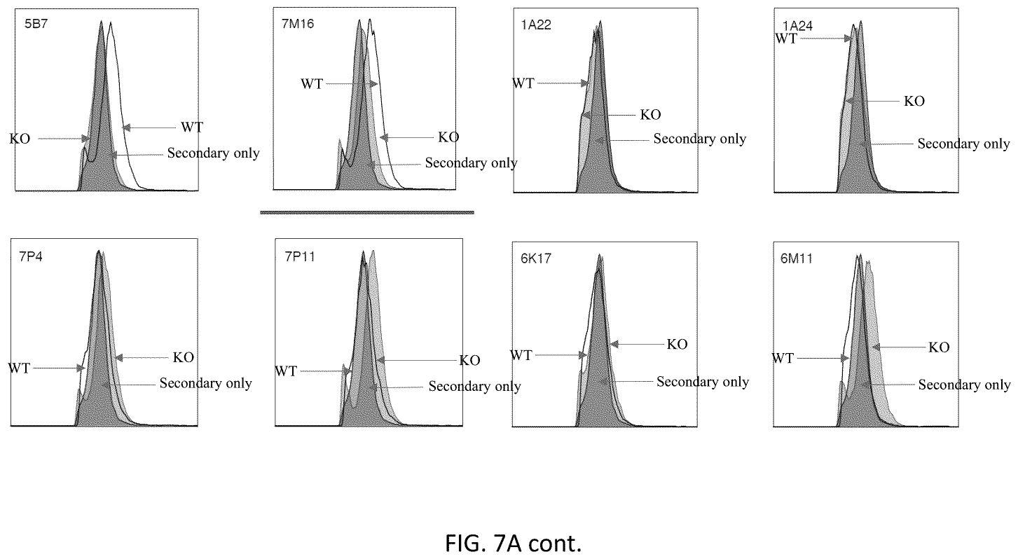

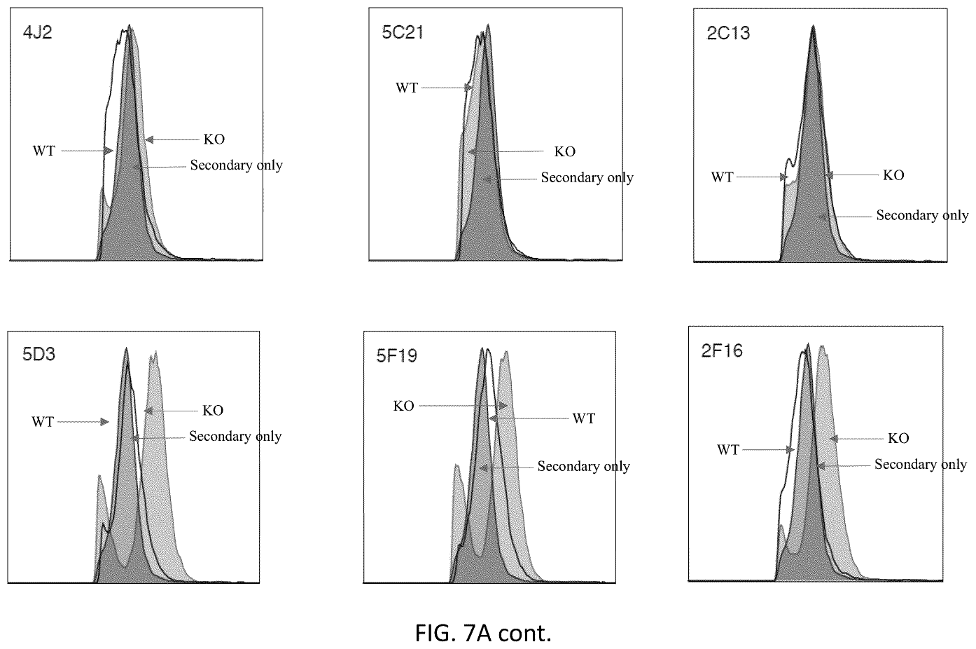

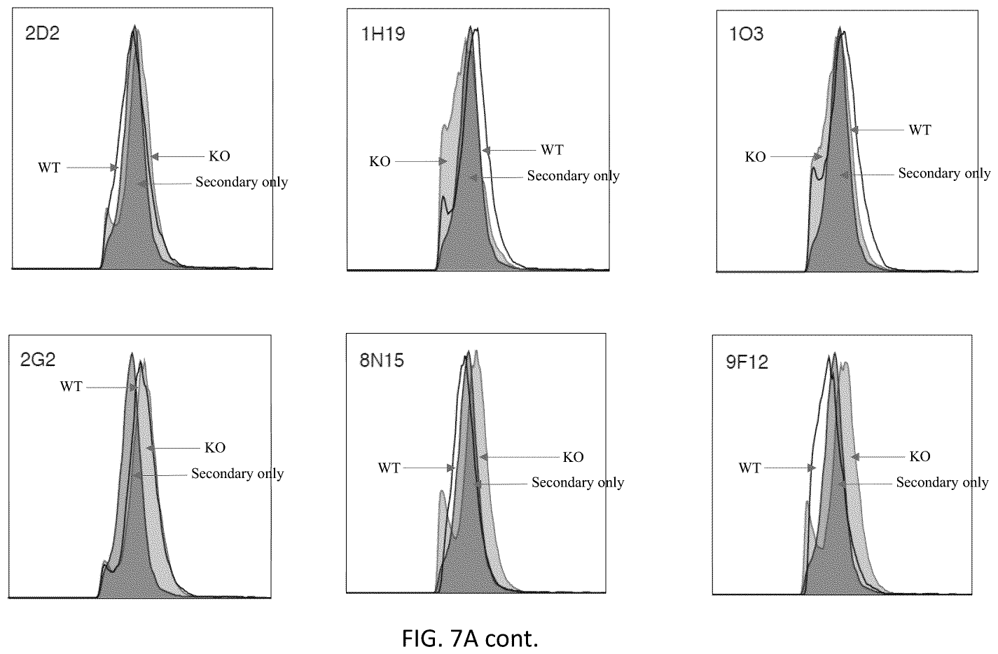

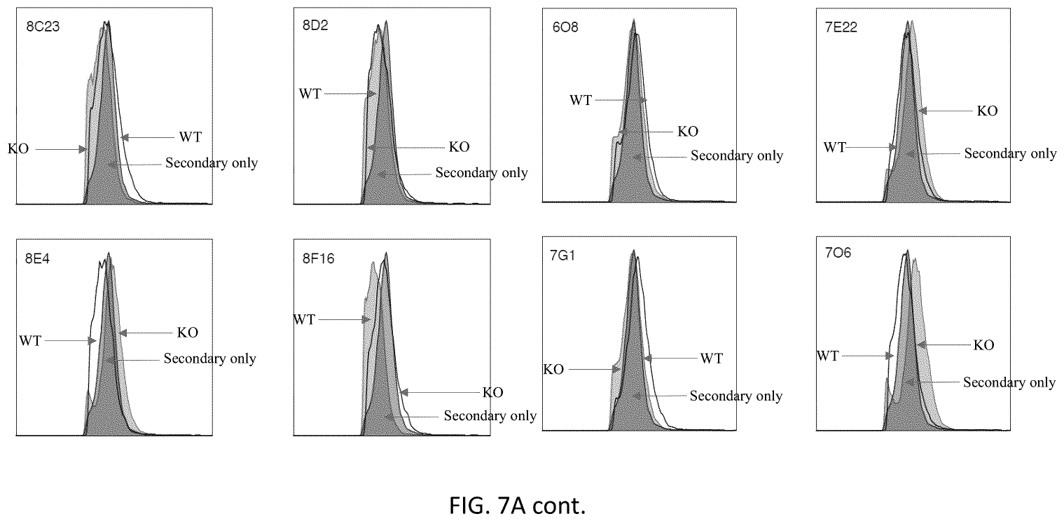

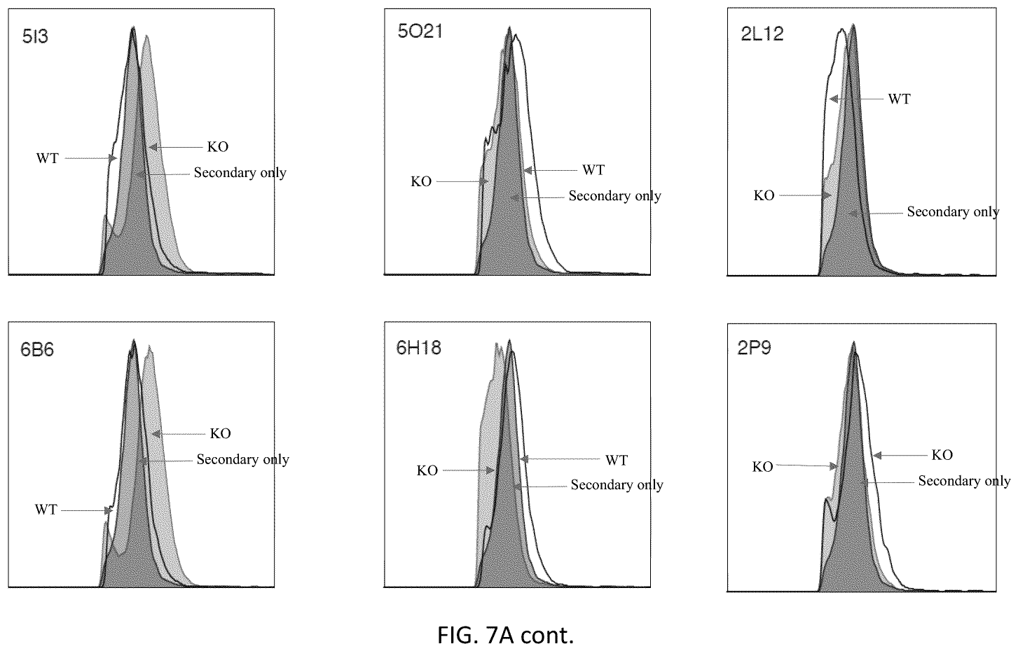

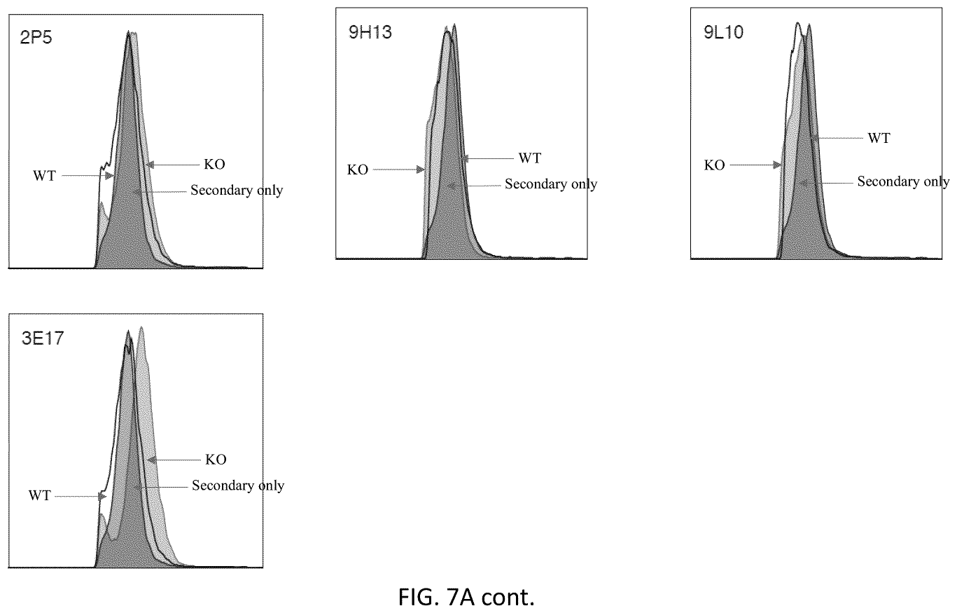

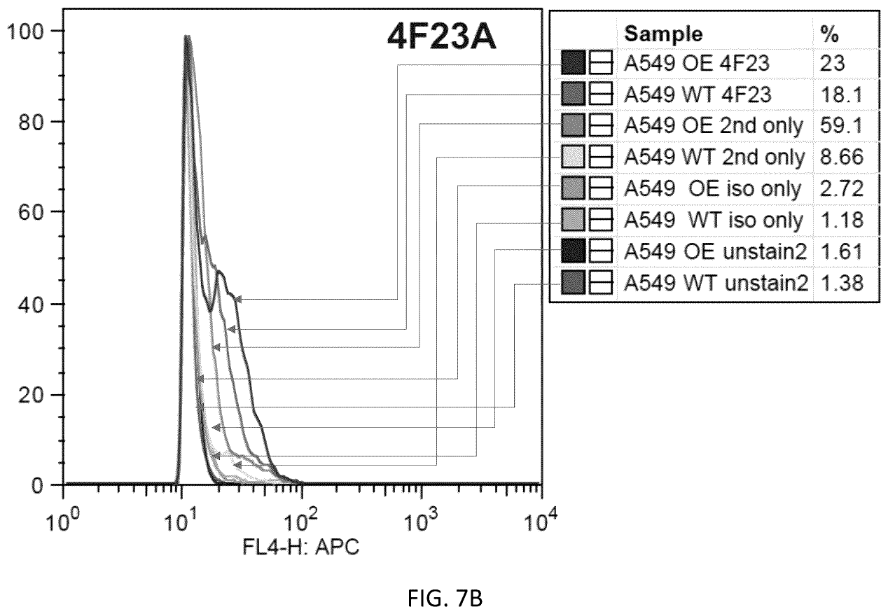

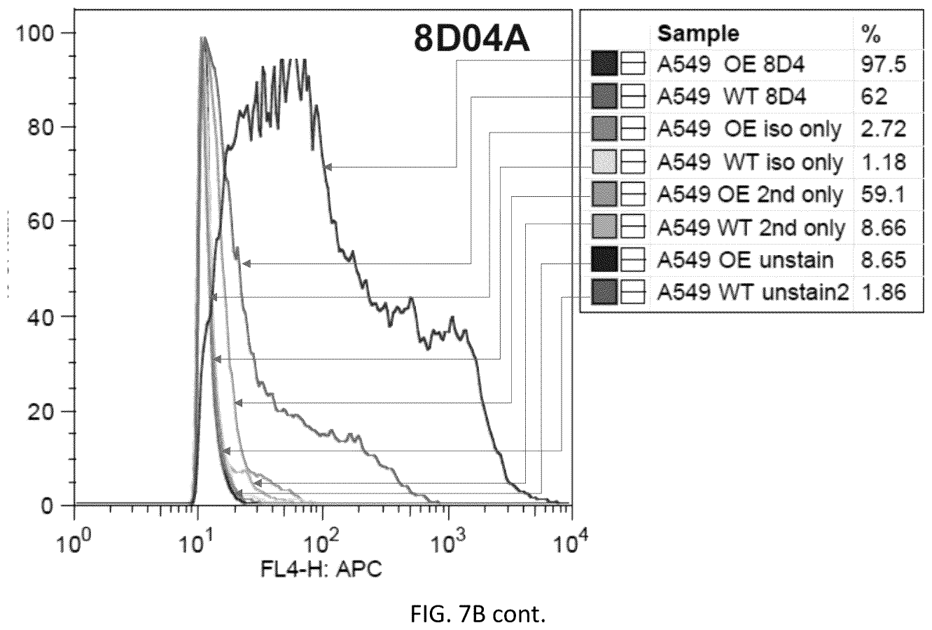

Following clonal expansion of the hybridoma library hybridoma supernatants were screened by ELISA against human, mouse, and cynomolgus PAG peptides, and narrowed in on 2 human binding clones and 1 human non-binding clone (FIG. 2A). To test the ability of the hybridoma supernatants to bind PAG on intact cells, splenocytes were stained from WT or PAG KO mice (FIG. 7A). 7M16A stained WT but not PAG KO splenocytes (FIG. 2B), suggesting specificity for PAG and the ability to bind endogenously presented PAG protein. To determine if clone 7M16A binds to the extracellular domain of human PAG on intact cells, A549 cells were generated that stably express PAG-GFP for this secondary screen. The level of PAG-GFP expression was confirmed by western blot (FIG. 2C), and that it is expressed at the plasma membrane (FIG. 2D). Then non-permeabilized A549 cells were stained with clones 7M16A, 4F23A, and 8D04A. PAG-specific surface was stained with 7M16A by flow cytometry (FIG. 7B; FIG. 2E). Protein binding was then confirmed by a dose-response ELISA, and found that clone 7M16A binds to human PAG while clones 8D04A and 4F23A were found not to bind (FIG. 2F).

To test the functional impact of 7M16A, primary human CD3 T cells were stimulated with anti-CD3 antibody and anti-CD28 antibody alone or in combination with 7M16A anti-PAG antibody and quantified IL-2 secretion over 48 hours. Given that PAG is inhibitory to T-cell activation, the neutralization of PAG is expected to lead to enhanced T-cell activation, and accordingly, the addition of 7M16A increases IL-2 secretion beyond anti-CD3 and anti-CD28 antibodies alone (FIG. 2G). To determine how 7M16A binding impacts the enrichment of PAG and PD-1 at the immune synapse both PAG-GFP and PD-1-SNAP+SNAP-AF647 were stably expressed in Jurkat T-cells (PD-1-SNAP-AF647) to visualize the localization of these proteins. These cells were pre-treated with 7M16A prior to co-culture with Raji B cells. PAG-GFP was enriched at the immune synapse in the majority of cells. However, when 7M16A is introduced PAG-GFP is excluded from the center of the immune synapse in the majority of cells (FIG. 2H). When both proteins were co-expressed, PD-1-SNAP-AF647 was also enriched outside of the immune synapse in the majority of cells (FIG. 2I).

These experiments show that clone 7M16A (Tables 1-3) binds to PAG, interfering with its localization and inhibitory function in vitro.

### Binding PAG and PD-1 Limits Tumor Growth and Enhances T-Cell Infiltration

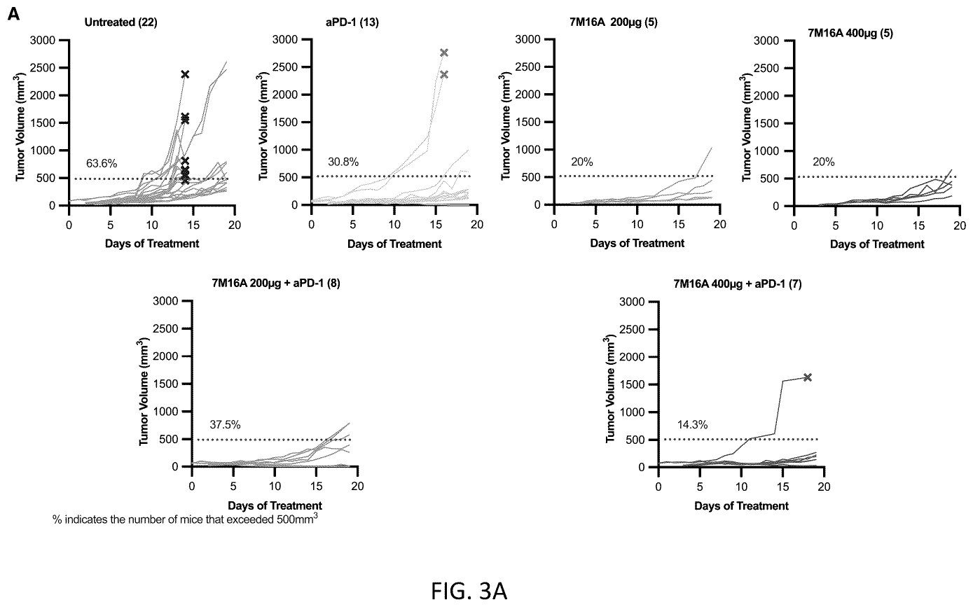

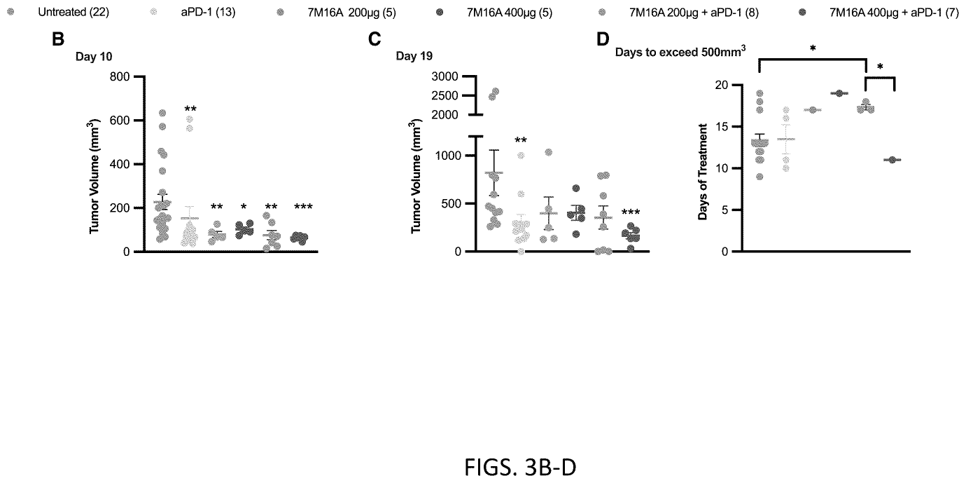

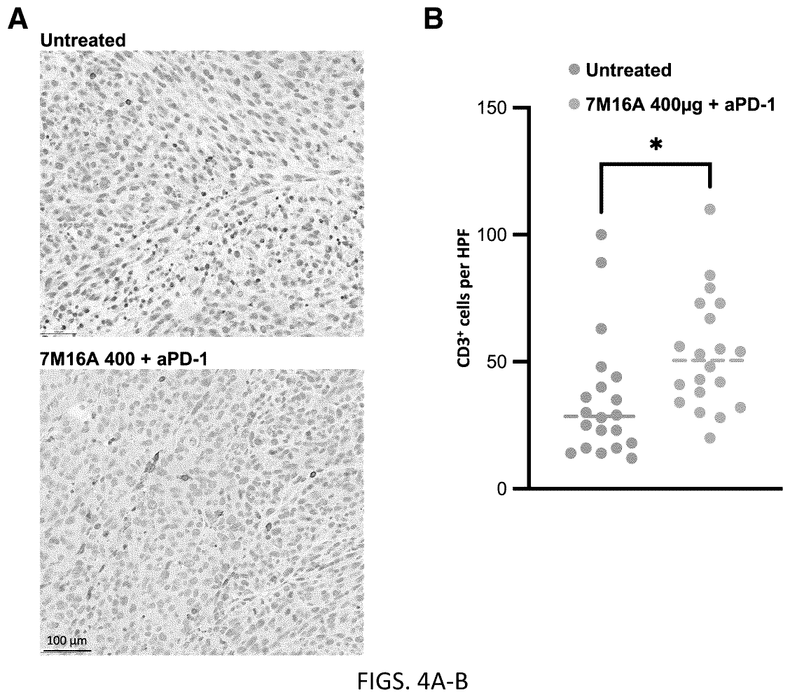

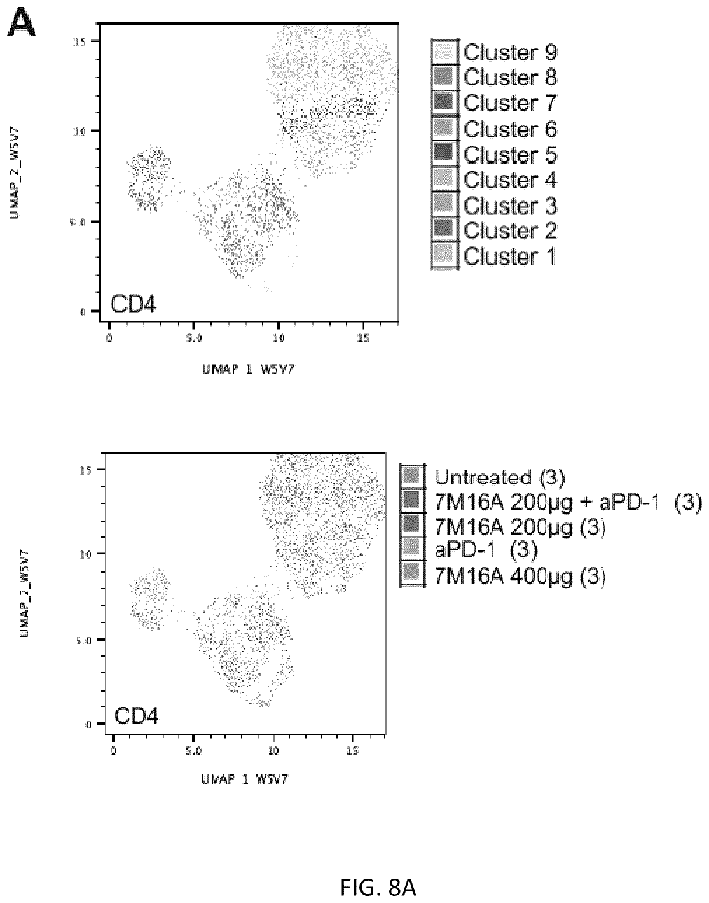

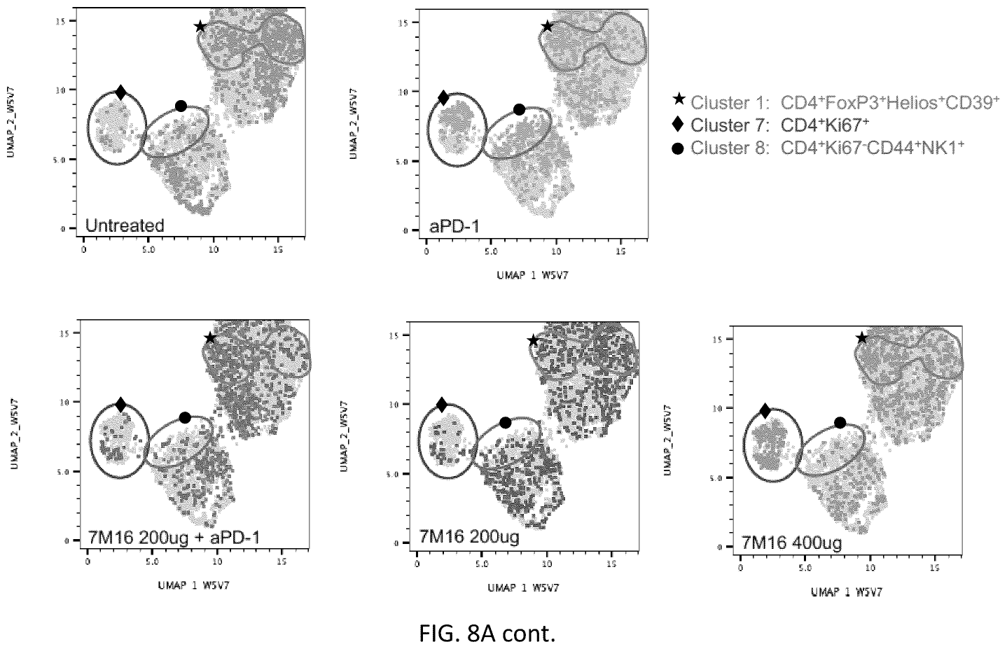

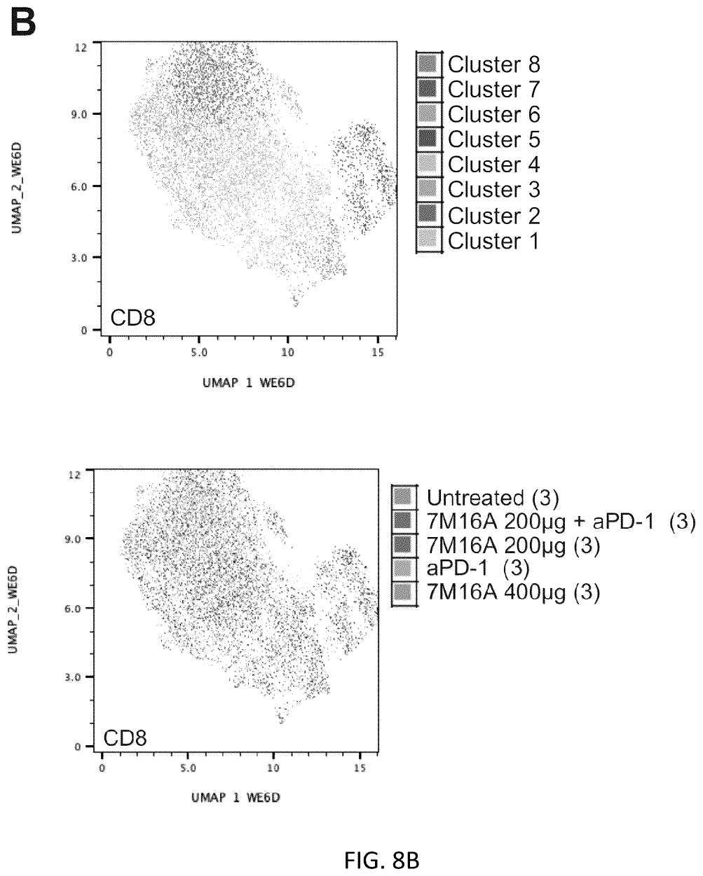

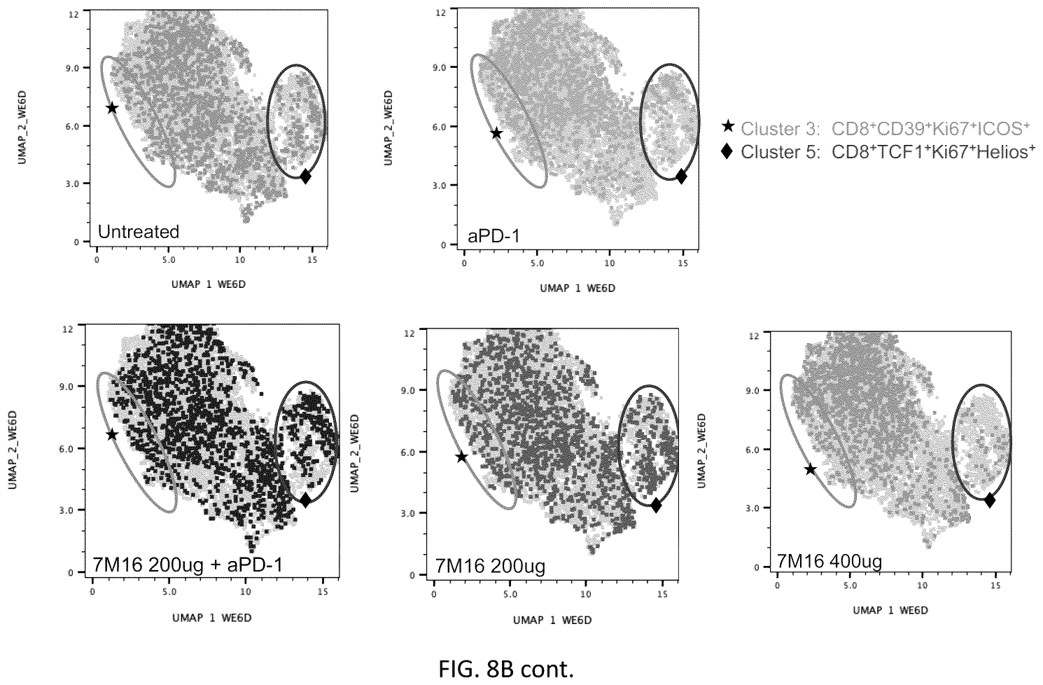

Two murine tumor models (MC38 and B16F10) were previously shown to exhibit limited growth and enhanced T-cell infiltration in the absence of PAG, suggesting that PAG is contributing to limited T-cell immune response. See Ref. 3. To determine if systemic administration of anti-PAG antibody alone or in combination with anti-PD-1 antibody impacts tumor growth the MC38 murine tumor model were utilized. First, MC38 murine colon adenocarcinoma cells were implanted subcutaneously and tumor growth were monitored daily until tumors reached 60-75 mm³. At this point, antibodies were delivered through intraperitoneal injection every four days for a total of four doses. The combined administration of anti-PAG clone 7M16A and anti-PD-1 antibodies resulted in inhibited tumor growth relative to untreated and anti-PD-1 alone (FIG. 3A). Additionally, tumors from mice treated with 7M16A alone or in combination demonstrated delayed growth kinetics (FIGS. 3B and 3C), with a longer growth time for the few tumors that do exceed 500 mm² (FIG. 3D). Immunohistochemistry of these tumors reveals increased CD3⁺ T cell infiltrates in the tumors from mice treated with anti-PAG clone 7M16A and anti-PD-1 compared with untreated mice (FIGS. 4A and 4B). More indepth immune phenotyping of the tumor infiltrating lymphocytes suggests additional differences in CD4⁺Ki67-CD44⁺NK1⁺ and CD8⁺TCF1⁺Ki67⁺Helios⁺ T cell subset distribution secondary to 7M16A and anti-PD-1 treatment (FIGS. 8A-B).

PAG expression within tumors has been previously identified to correlate negatively with patient outcome in colon adenocarcinoma, renal cell carcinoma, melanoma, acute myeloid leukemia, invasive breast, cervical squamous, and testicular cancers. See Refs 3, 7-11. Additionally, it was demonstrated that PAG is phosphorylated downstream of PD-1 engagement, and that genetic deletion of PAG in mice limits tumor growth. See Ref. 3. During T cell-APC interaction, PAG is enriched at the immune synapse. The exclusion of molecules from the immune synapse by size has been previously reported. See Refs. 12, 13. In one detailed study using a range of sizes of dextran molecules, it was established that the movement of dextran molecules ≤4 nm in and out of the immune synapse was unrestricted, but the movement of 10-13 nm dextran molecules was greatly reduced, and dextran molecules above 32 nm were nearly completely excluded. See Ref. 14. This study went further to show that monoclonal antibodies are excluded from the immune synapse. See Ref. 14. Hence, a strategy to target PAG localization to alter PD-1 signaling and T cell function was designed.

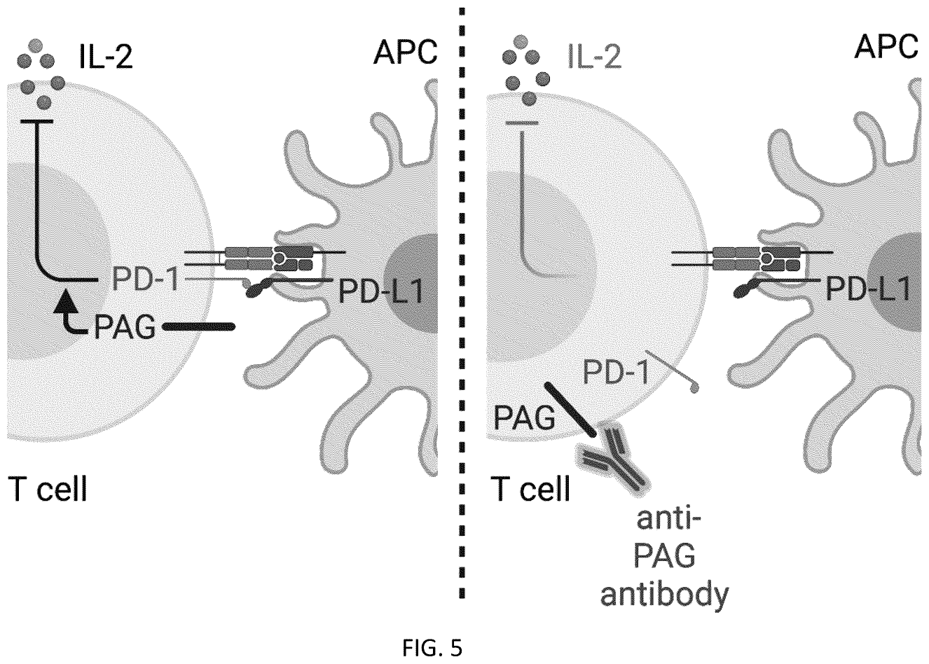

Described herein is the application of the principle of size exclusion to describe a novel strategy of using a monoclonal antibody to change the localization of a T cell surface protein in the context of the immune synapse (FIG. 5). Antibodies have been previously shown to alter the localization of a protein on the cell surface, and in fact the synaptic autoimmune disorder anti-N-methyl-D-aspartate receptor (NMDAR) encephalitis is characterized by auto-antibodies that target NMDAR in the brain and cause removal of the receptor from the neurological synapse. See Ref. 15. When a T cell and APC come together, forming the immune synapse, the precise localization of each protein contributes to overall signaling and function. Overall, the immune synapse architecture can be broken down into circular regions referred to as the central, peripheral, and distal supramolecular activation clusters (SMACs), and the relative proximity between individual proteins within these regions is critical to cellular function. See Ref. 16. Leveraging the strict organization of the immune synapse for immunotherapeutic intervention is currently of high interest in the field, and numerous approaches are being explored. See Ref. 17.

Through this study, an antibody that binds human and mouse PAG and alters murine tumor growth in combination with anti-PD-1 was successfully generated and purified. Of note, this study of murine tumor growth was designed to establish the ability of this anti-PAG antibody to alter tumor growth and enhance anti-PD-1 response in a situation of poor response to anti-PD-1 antibody alone. For this reason, we initiated anti-PAG antibody administration prior to the previously published (see Ref. 3) three-dose, 200 mg regimen of anti-PD-1 antibody, therefore increasing the tumor size at initiation of anti-PD-1 antibody and somewhat diminishing the MC38 response to anti-PD-1 antibody.

This PAG antibody has been humanized. The humanized antibody is tested for function in a humanized mouse model along with human anti-PD-1 to test efficacy as well as safety.

### Materials and Methods

### General Reagents

Roswell Park Memorial Institute (RPMI; Cat. No. SLM-140) 1640 medium, Dulbecco's modified Eagle's medium (DMEM; Cat. No. SLM-020), Dulbecco's phosphate-buffered saline (DPBS), penicillin/streptomycin (Cat. No. P4333), and fetal bovine serum (FBS; Cat. No. F4135) were purchased from Life Technologies. Lymphoprep was purchased from Stemcell Technologies (Cat. No. 07811). Superantigen Staphylococcal enterotoxins (SEE) was purchased from Toxin Technology (Cat. No. ET404).

### DNA Constructs

peGFP-N1-PAG and peGFP-N1-FC-PAG constructs were generated by PCR sub-cloning and site-directed mutagenesis (Agilent, Cat. No. 210518). pCDH-PAG-GFP was generated by PCR cloning. PHR-PD-1-SNAP was generously provided by Enfu Hui (University of California, San Diego).

### Antibodies

Antibodies used include those against RhoGDI (polyclonal, Abcam, Cat. No. ab175243), EGFR (clone EP38Y, Abcam, Cat. No. ab52894), PD-1 (clone D4W2J, Cell Signaling, Cat. No. 86163T), and PAG (clone MEM-255, Origene, Cat. No. SM3069P).

### Cell Culture and Transfection

Primary murine splenocytes were isolated by mechanical disruption of spleens from 10-12-week-old mice to generate a single cell suspension. A549 human lung epithelial carcinoma cell line and HEK 293T human embryonic kidney epithelial cells (ATCC, Cat. Nos. CRM-CCL-185; CRL-3216) were maintained in 5% CO₂ at 37° C. in DMEM supplemented with 10% FBS and 1% penicillin/streptomycin. Jurkat and Raji cells were obtained from the American Type Culture Collection (ATCC, Cat. Nos. TIB-152; CCL-86). These cell lines were maintained in 5% CO₂ at 37° C. in RPMI 1640 supplemented with 10% FBS and 1% penicillin/streptomycin. Raji and Jurkat cells were used in co-culture assays stimulated with SEE (2 μg/mL). For localization experiments, live cell co-cultures were imaged for 30 min by confocal microscopy (Zeiss LSM 900). To generate stable PAG-GFP expressing Jurkat T cells and A549 cells, first HEK 293T cells were transfected with lentiviral envelope and packaging plasmids pMD2G and psPAX2, respectively, along with pCDH-PAG-GFP to generate PAG containing lentiviral particles. The collected lentiviral particles were then used to transduce Jurkat T cells or A549 cells, and puromycin (2 μg/mL) resistance was used for selection. Stable PAG-GFP expressing Jurkat cells were then used to generate cells co-expressing PAG-GFP and PD-1-SNAP using the same process.

### Immunoprecipitation and Mass Spectrometry

A549 cells stably expressing PAG-GFP were lysed in cold RIPA lysis buffer, containing complete Mini, EDTA-free protease inhibitors (Roche). The cells were placed on a rotator and lysis was carried at 4° C. for 30 min. The lysates were centrifuged for 10 min at 12,000 g and 4° C. Lysates were then used for immunoprecipitation of PAG-GFP using anti-GFP antibody conjugated agarose beads (MBL Cat. No. D153-8) according to the manufacturer's protocol. The PAG-GFP enriched protein lysates were then separated by Tris-glycine PAGE, stained with Coomassie Brilliant blue, and the band at the correct size was cut. The gel was treated with chymotrypsin and the enriched proteins were identified by Fusion Tribrid mass spectrometer and analyzed with Scaffold 4.0 software.

### Western Blotting

If stimulated, Jurkat T cells were stimulated with plate-bound 5 μg/mL anti-CD3 antibody (clone UCHT1) and 1 μg/mL anti-CD28 antibody (clone CD28.2) for 18 hours. Cells were then collected and placed on ice, resuspended in ice cold PBS and centrifuged for 5 min at 400 g and 4° C. The cell pellets were resuspended in cold RIPA lysis buffer, containing complete Mini, EDTA-free protease inhibitors (Roche). The cells were placed on a rotator and lysis was carried at 4° C. for 30 min. The lysates were centrifuged for 10 min at 12,000 g and 4° C. Where fractionation is noted, clarified cell lysates were fractionated by ultracentrifugation at 100,000×g. The resulting supernatant (S100) contains soluble, cytosolic proteins and the pellet (P100) contains cell membrane associated proteins. Samples were prepared with 2× Laemmli buffer, boiled at 95° C. for 10 min and run on SDS-PAGE. Following protein transfer for 30 min at 25V, the nitrocellulose membrane was blocked with 5% bovine serum albumin (BSA) in PBS containing 0.05% Tween-20 (PBST) and blotted overnight with primary antibody prepared in PBST containing 2.5% BSA. The membrane was developed with IRDye secondary fluorescent antibody and acquired on Odyssey CLx Imaging system.

### Proximity Ligation Assay

The proximity ligation assay (PLA) kit, Duolink In Situ Red was obtained from Sigma-Aldrich (Cat. No. DUO92101-1KT). WT Jurkat T cells were treated with 2 μg/mL PHA overnight to induce PD-1 expression. The next day, PD-1 expression was confirmed by flow cytometry, and cells were resuspended in OPTI-MEM. Coverslips were washed and coated with 0.01% poly-L-lysine for 5 min at room temperature. Jurkat cells were added to the slide and allowed to adhere for 30 min, while Raji B cells were pre-loaded with 5 μg/mL SEE in OPTI-MEM for 30 m, both at 5% CO₂ at 37° C. Without washing, Raji cells were added to the coverslip at 1:1 ratio. Cells were incubated for 30 min at 5% CO₂ at 37° C. to allow immune synapses to form. Cells were then fixed with 4% paraformaldehyde for 10 min and permeabilized with 0.2% Triton-X100 for 10 min. Finally, cells were subjected to the manufacturer's protocol for Duolink PLA. Images were acquired using a Zeiss LSM 900 and analyzed using ImageJ (NIH).

### Immunization Strategy

Balb/C and B6-129 mice were immunized with 1-16 AA human PAG-KLH to stimulate antibody production. Antibody-secreting plasma cells were then isolated from mice with positive serum binding to peptide in the first ELISA screen, and fused with myeloma cells to generate hybridomas. Hybridomas were selected in HAT medium and sorted into clones. Clones were selected based on ELISA (primary) and flow cytometry (secondary) screening for positive binders. Selected hybridoma clones were then expanded to produce purified monoclonal antibodies.

### IL-2 ELISA

To determine the concentration of secreted IL-2 following 48 hour antibody stimulation, the human IL-2 ELISA kit (BioLegend, Cat. No. 570409) was used according to the manufacturer protocol. Primary human CD3⁺ T cells were purified from peripheral blood by RosetteSep CD3⁺ Negative Selection (Stemcell, Cat. No. 15021) followed by Lymphoprep separation. T cells were stimulated with plate-bound anti-CD3 (clone UCHT1) and anti-CD28 (clone CD28.2) antibodies (10 μg/mL) and anti-PAG antibody clone 7M16A (50 μg/mL) for 48 hours prior to supernatant collection and ELISA.

### PAG ELISA

Primary ELISA was done for screening binding of hybridoma supernatants to human, murine, and cynomolgus PAG (2 μg/mL). Second ELISA for confirmation of binding of purified monoclonal antibodies to human PAG, 96-well half area high binding ELISA plates (Corning 3690) were coated 1 μg/mL of human PAG protein in 1×TBS at room temperature for 2 hours. The plate was washed with TBS containing 0.05% Tween 20 and blocked with 2% BSA in TBS for 1 hour at room temperature. After 1 hour, the plate was washed and diluted PAG antibodies in 1×TBS were dispensed to respective wells and incubated for 1 hour at room temperature. The plate was washed and incubated with peroxidase-Affinipure F(ab′)2 goat anti-mouse IgG (Jackson Immuno Research, Cat. No. 115-006-003) at 1:5000 dilution for 1 hour at room temperature. After the plate was washed, 3, 3′, 5, 5′ tetramethyl benzidine (TMB) substrate (Biolegend, Cat. No. 421101) was added to each well. The reaction was stopped by adding 2N H2SO4. Absorbance was measured on plate reader (Biotek Synergy H1) and results calculated by A=A450 nm-A570 nm.

### Flow Cytometry

Non-permeabilized cells were stained with the primary antibody clones in FACS Buffer [HBSS without Ca²⁺/Mg²⁺, FBS (3%), NaN3 (0.02%), and CaCl2 (2.5 mM)], then washed and stained with fluorescently conjugated secondary antibody (goat anti-mouse, Jackson ImmunoResearch, Cat. No. 115-006-003). Stained cells were then fixed in 1% paraformaldehyde. Splenocytes were pre-treated with Fc receptor block (Biolegend, Cat. No. 422301). PAG-GFP expressing cells were fixed in 1% paraformaldehyde. Events were recorded using FACSCanto (BD), and analyzed using FlowJo software (Ver. 10.8.1).

### Raji-Jurkat Conjugate Microscopy

Raji cells were stained with Tag-It Violet (BioLegend) according to the manufacturer protocol then pre-incubated with SEE (100 ng/ml) for 1 hour at 5% CO₂ at 37° C. When PD-1-SNAP expressing Jurkat cells were included in the assay, the cells were pre-stained with SNAP-AF-647 (NEB) according to manufacturer protocol. When 7M16A was included, Jurkat T cells expressing PAG-GFP or PAG-GFP and PD-1-SNAP were pre-incubated with 7M16A (1 μg/mL) for 1 hour at 5% CO₂ at 37° C. Both Raji and Jurkat cells were then individually diluted to 4E6 cells/mL in serum free media and combined in a 1:1 ratio immediately before imaging on poly-L-lysine coated image plates (Mattek). Conjugates were then imaged continuously for 30 min. Images were acquired using a Zeiss LSM 900 and analyzed using ImageJ (NIH).

### Mice and Tumor Cell Lines

The Columbia University institutional Animal Care approved animal studies and use committee (IACUC). Balb/C, B6-129 mice used for immunizations were sourced by LakePharma. Male, 6-12-week-old C57BL/6 (B6) wild-type (WT) or PAG knock-out (PAG KO) 8 mice were used in all animal studies. All studies' WT and PAG KO mice were littermates, assuring a homogeneous genetic background. The murine colon adenocarcinoma (MC38) colon carcinoma cells were a gift from Ben Neel of New York University. Prior to use, MC38 cells were authenticated by simple sequence length polymorphism (SSLP). The MC38 cells were maintained in DMEM supplemented with heat-inactivated fetal bovine serum (FBS; 10%) and penicillin-streptomycin (P/S; 1% 10,000 U/ml stock) and grown at 37° C. with 5% CO₂. Cells were passaged prior to storage and thawed and passaged twice prior to implantation for all described tumor experiments. All cell lines were determined to be free of Mycoplasma (Lonza, Cat. No. LT07-318).

### Tumor Models

MC38 (1×10⁶) cells were implanted subcutaneously in the right hind flank of 6-10-week-old mice. Tumor growth was monitored using electronic calipers and calculated according to the formula: V=length×width²×0.52. When tumor volume reached 60-70 mm³ the first dose of 7M16A was administered by IP injection (treatment day 0). 7M16A was then administered every 4^{th} day for a total of 4 doses. Anti-PD-1 (200 μg; BioXcell clone RPM1-14) was initiated on treatment day 4 and administered by IP injection every 4^{th} day for a total of 3 doses. For immunohistochemistry, tumors were fixed in 10% neutral buffered formalin then paraffin embedded and cut into 5 μm sections. Slices were stained with anti-CD3 (Clone SP7, Abcam, Cat. No. ab16669), and bound antibody was detected with peroxidase-based staining.

### Tumor-Infiltrating Lymphocyte Flow Cytometry

MC38 (1×10⁶) cells were implanted subcutaneously in the right hind flank of 6-10-week-old mice. Tumor growth was monitored using electronic calipers and calculated according to the formula: V=length×width²×0.52. When tumor volume reached 40-50 mm³ the first dose of 7M16A was administered by IP injection (treatment day 0). 7M16A was then administered every 4^{th} day for a total of 3 doses. Anti-PD-1 (200 μg; BioXcell clone RPM1-14) was initiated on treatment day 4 and administered by IP injection every 4^{th} day for a total of 2 doses. Tumors were harvested on day 14 for single cell isolation. Harvested tumors were diced into small pieces and incubated in digestion media containing Collagenase H (Sigma, C8051) and Dnase (Roche, 10104159001) in RPMI at 37 degrees for 30 min. Following digestion, tumors were pressed through 70 μm mesh strainers to generate a single-cell suspension. Cells were then overlaid on Lymphoprep (Stemcell) and centrifuged at 800 g to 10 min without break to remove any dead cells. Cells were then collected from the cell interface and stained for flow cytometry. Following FC receptor block, cells were stained for surface proteins with fluorescently conjugated antibodies (TCR-B, CD103, CD44, PD-1, Nrp1, CD4, CD39, IA-IE, ST2, CD8, CD62L, CD11c, ICOS, CXCR3, KLRG1, PD-L1, CD45, Sca-1, Ly6C, CD206, NK1.1, B220, CD69, CD11b, F4/80, CD38) then fixed and permeabilized (Biolegend, True-Nuclear Transcription Factor Buffer Set) for intracellular staining (Ki67, iNOS, TOX, Ly6G, Helios, FoxP3, TCF-1). Events were recorded using Cytek Aurora 5L, and analyzed using FlowJo software (Ver. 10.8.1).

### Statistics

Values are reported as mean±SEM. Statistical analyses were performed using Mann-Whitney test. All statistical analyses were performed using GraphPad Prism 9.

### REFERENCES FOR EXAMPLE 1

1. Carretero-González A., Lora D., Ghanem I., Zugazagoitia J., Castellano D., Sepúlveda J. M., López-Martin J. A., Paz-Ares L., de Velasco G. Analysis of response rate with ANTI PD1/PD-L1 monoclonal antibodies in advanced solid tumors: a meta-analysis of randomized clinical trials. Oncotarget. 2018; 9:8706-8715. doi: 10.18632/oncotarget.24283.
2. Yarchoan M., Hopkins A., Jaffee E. M. Tumor mutational burden and response rate to PD-1 inhibition. N. Engl. J. Med. 2017; 377:2500-2501. doi: 10.1056/NEJMc1713444.
3. Strazza M., Azoulay-Alfaguter I., Peled M., Adam K., Mor A. Transmembrane adaptor protein PAG is a mediator of PD-1 inhibitory signaling in human T cells. Commun. Biol. 2021; 4:672. doi: 10.1038/s42003-021-02225-8.
4. Néron B., Mënager H., Maufrais C., Joly N., Maupetit J., Letort S., Carrere S., Tuffery P., Letondal C. Mobyle: a new full web bioinformatics framework. Bioinformatics. 2009; 25:3005-3011. doi: 10.1093/bioinformatics/btp493.
5. Kyte J., Doolittle R. F. A simple method for displaying the hydropathic character of a protein. J. Mol. Biol. 1982; 157:105-132. doi: 10.1016/0022-2836(82)90515-0. [PubMed] [CrossRef] [Google Scholar]
6. Hrdinka M., Horejsi V. PAG—a multipurpose transmembrane adaptor protein. Oncogene. 2014; 33:4881-4892. doi: 10.1038/onc.2013.485. [PubMed] [CrossRef][Google Scholar]
7. Ashburner M., Ball C. A., Blake J. A., Botstein D., Butler H., Cherry J. M., Davis A. P., Dolinski K., Dwight S. S., Eppig J. T., et al. Gene ontology: tool for the unification of biology. The Gene Ontology Consortium. Nat. Genet. 2000; 25:25-29. doi: 10.1038/75556.
8. Dobenecker M. W., Schmedt C., Okada M., Tarakhovsky A. The ubiquitously expressed Csk adaptor protein Cbp is dispensable for embryogenesis and T-cell development and function. Mol. Cell Biol. 2005; 25:10533-10542. doi: 10.1128/MCB.25.23.10533-10542.2005.
9. Draberova L., Bugajev V., Potuckova L., Halova I., Bambouskova M., Polakovicova I., Xavier R. J., Seed B., Draber P. Transmembrane adaptor protein PAG/CBP is involved in both positive and negative regulation of mast cell signaling. Mol. Cell Biol. 2014; 34:4285-4300. doi: 10.1128/MCB.00983-14.
10. Hayward N. K., Wilmott J. S., Waddell N., Johansson P. A., Field M. A., Nones K., Patch A. M., Kakavand H., Alexandrov L. B., Burke H., et al. Whole-genome landscapes of major melanoma subtypes. Nature. 2017; 545:175-180. doi: 10.1038/nature22071. [PubMed] [CrossRef] [Google Scholar]
11. Tedoldi S., Paterson J. C., Hansmann M. L., Natkunam Y., Rüdiger T., Angelisova P., Du M. Q., Roberton H., Roncador G., Sanchez L., et al. Transmembrane adaptor molecules: a new category of lymphoid-cell markers. Blood. 2006; 107:213-221. doi: 10.1182/blood-20-06-2273. [PubMed] [CrossRef] [Google Scholar]
12. Hartman N. C., Nye J. A., Groves J. T. Cluster size regulates protein sorting in the immunological synapse. Proc. Natl. Acad. Sci. USA. 2009; 106:12729-12734. doi: 10.1073/pnas.0902621106.
13. Alakoskela J. M., Koner A. L., Rudnicka D., Köhler K., Howarth M., Davis D. M. Mechanisms for size-dependent protein segregation at immune synapses assessed with molecular rulers. Biophys. J. 2011; 100:2865-2874. doi: 10.1016/j.bpj.2011.05.013.
14. Cartwright A. N. R., Griggs J., Davis D. M. The immune synapse clears and excludes molecules above a size threshold. Nat. Commun. 2014; 5:5479. doi: 10.1038/ncomms6479.
15. Kayser M. S., Dalmau J. Anti-NMDA receptor encephalitis, autoimmunity, and psychosis. Schizophr. Res. 2016; 176:36-40. doi: 10.1016/j.schres.2014.10.007.
16. Dustin M. L. The immunological synapse. Cancer Immunol. Res. 2014; 2:1023-1033. doi: 10.1158/2326-6066. CIR-14-0161.
17. Finetti F., Baldari C. T. The immunological synapse as a pharmacological target. Pharmacol. Res. 2018; 134:118-133. doi: 10.1016/j.phrs.2018.06.009. [PubMed] [CrossRef] [Google Scholar]
18. Dragovich M. A., Adam K., Strazza M., Tocheva A. S., Peled M., Mor A. SLAMF6 clustering is required to augment T cell activation. PLOS One. 2019; 14: e0218109. doi: 10.1371/journal.pone.0218109.
19. Felce J. H., Sezgin E., Wane M., Brouwer H., Dustin M. L., Eggeling C., Davis S. J. CD45 exclusion- and cross-linking-based receptor signaling together broaden FcepsilonRI reactivity. Sci. Signal. 2018; 11: eaat0756. doi: 10.1126/scisignal.aat0756.
20. Fernandes R. A., Su L., Nishiga Y., Ren J., Bhuiyan A. M., Cheng N., Kuo C. J., Picton L. K., Ohtsuki S., Majzner R. G., et al. Immune receptor inhibition through enforced phosphatase recruitment. Nature. 2020; 586:779-784. doi: 10.1038/s41586-020-2851-2.
21. Dimitriou F., Matter A. V., Mangana J., Urosevic-Maiwald M., Micaletto S., Braun R. P., French L. E., Dummer R. Cytokine release syndrome during sequential treatment with immune checkpoint inhibitors and kinase inhibitors for metastatic melanoma. J. Immunother. 2019; 42:29-32. doi: 10.1097/CJI.0000000000000236. [PubMed] [CrossRef][Google Scholar]
22. Lee D. W., Gardner R., Porter D. L., Louis C. U., Ahmed N., Jensen M., Grupp S. A., Mackall C. L. Current concepts in the diagnosis and management of cytokine release syndrome. Blood. 2014; 124:188-195. doi: 10.1182/blood-2014-05-552729.
23. Gao C., Xu J., Han C., Wang L., Zhou W., Yu Q. An esophageal cancer case of cytokine release syndrome with multiple-organ injury induced by an anti-PD-1 drug: a case report. Ann. Palliat. Med. 2020; 9:2393-2399. doi: 10.21037/apm-20-1310. [PubMed] [CrossRef] [Google Scholar]
24. Poussin M., Sereno A., Wu X., Huang F., Manro J., Cao S., Carpenito C., Glasebrook A., Powell D. J., Jr., Demarest S. Dichotomous impact of affinity on the function of T cell engaging bispecific antibodies. J. Immunother. Cancer. 2021; 9: e002444. doi: 10.1136/jitc-2021-002444.
25. Fang Y., Zhang Y., Guo C., Chen C., Gao H., Zhou X., Liu T., Qian Q. Safety and efficacy of an immune cell-specific chimeric promoter in regulating anti-PD-1 antibody expression in CAR T cells. Mol. Ther. Methods Clin. Dev. 2020; 19:14-23. doi: 10.1016/j.omtm.2020.08.008.
26. Wang C., Shi F., Liu Y., Zhang Y., Dong L., Li X., Tong C., Wang Y., Su L., Nie J., Han W. Anti-PD-1 antibodies as a salvage therapy for patients with diffuse large B cell lymphoma who progressed/relapsed after CART19/20 therapy. J. Hematol. Oncol. 2021; 14:106. doi: 10.1186/s13045-021-01120-3.

### Example 2

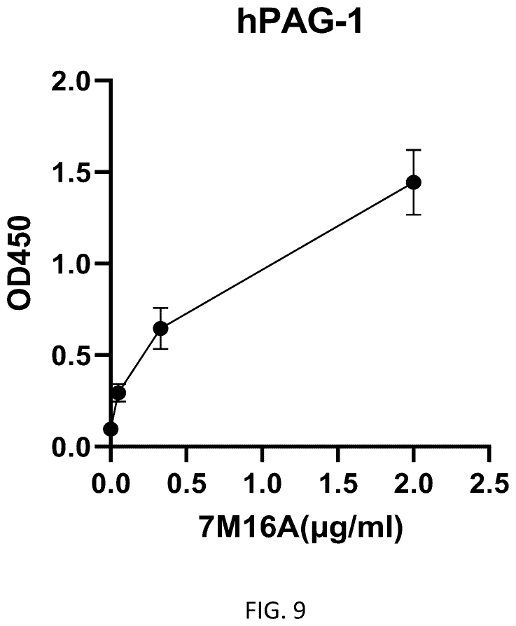

ELISA binding assays of the 7M16A clone to the human PAG-1 peptide were performed (FIG. 9).

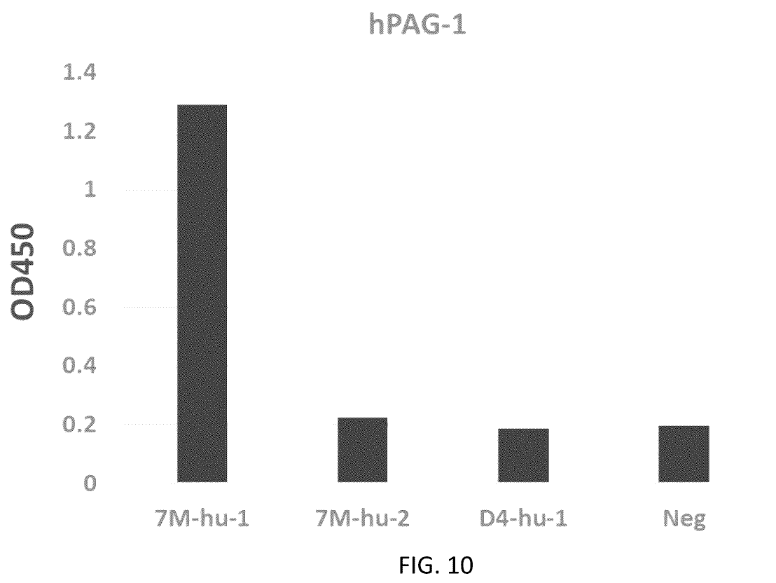

Humanized versions of the 7M16A were synthesized, denoted as 7M-hu-1 and 7M-hu-2. 7M-hu-1 comprise a VH domain comprising SEQ ID NO:27 and a VL domain comprising SEQ ID NO: 33. ELISA binding shows higher binding of human PAG-1 peptide to 7M-hu-1 compared to human PAG-1 binding to 7M-hu-2 (FIG. 10). D4-hu-1 and negative control show minimal binding to human PAG-1 peptide.

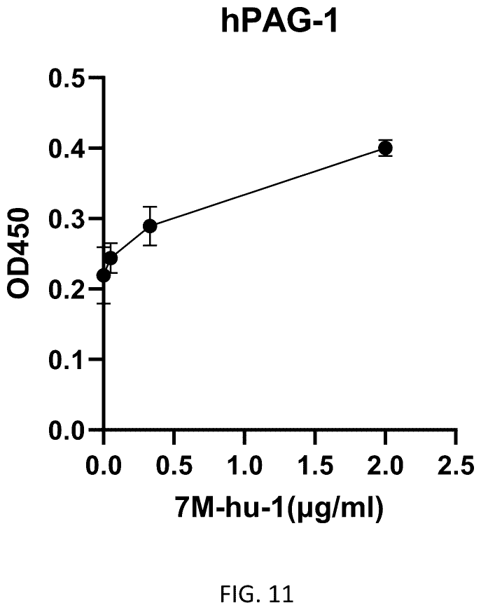

ELISA binding assays of the 7M-hu-1 to the human PAG-1 peptide were performed (FIG. 11).

What is claimed is:

1. A monoclonal antibody or a fragment thereof, comprising:
a first arm comprising a first variable heavy chain domain and a first variable light chain domain, wherein a portion of the first arm is capable of binding to an extracellular portion of human PAG; and
a second arm comprising a second variable heavy chain domain and a second variable light chain domain, wherein a portion of the second arm is capable of binding to an extracellular portion of human PAG wherein the first and second arms each further comprise a fragment, crystallizable (Fc) domain.

2. The monoclonal antibody of claim 1, wherein the first and second arms each further comprise a CH1 domain, a hinge domain, and a CL domain.

3. The monoclonal antibody of claims 1-2, wherein:
the first variable heavy chain domain of the first arm is encoded by a first polypeptide chain;
the first variable light chain domain of the first arm is encoded by a second polypeptide chain;
the second variable heavy chain domain of the second arm is encoded by a third polypeptide chain;
the second variable light chain domain of the second arm is encoded by a fourth polypeptide chain; and
the first variable heavy chain domain and first variable light chain domain form a first PAG binding site and wherein the second variable heavy chain domain and second variable light chain domain form a second PAG binding site.

4. The monoclonal antibody of claim 3, wherein the first and second PAG binding sites are the same.

5. The monoclonal antibody of claim 3, wherein the first and third polypeptide chain each further encode a hinge domain, a CH1 domain, and the Fc domain, and wherein the second and fourth polypeptide chain each further encode a CL domain.

6. The monoclonal antibody of claim 5, wherein the first and third polypeptide chains comprise the same sequence and the second and fourth polypeptide chains comprise the same sequence.

7. The monoclonal antibody of claims 1-6, wherein the first variable heavy chain domain comprises an amino acid sequence comprising SEQ ID NO: 5, 16, or 27, and wherein the first variable light chain domain comprises an amino acid sequence comprising SEQ ID NO: 11, 21, or 28.

8. The monoclonal antibody of claims 1-6, wherein the first arm comprises an amino acid sequence comprising SEQ ID NO: 5 and the second arm comprises an amino acid sequence comprising SEQ ID NO: 11, the first arm comprises an amino acid sequence comprising SEQ ID NO: 10 and the second arm comprises an amino acid sequence comprising SEQ ID NO: 15, the first arm comprises an amino acid sequence comprising SEQ ID NO: 16 and the second arm comprises an amino acid sequence comprising SEQ ID NO: 21, the first arm comprises an amino acid sequence comprising SEQ ID NO: 20 and the second arm comprises an amino acid sequence comprising SEQ ID NO: 25, or the first arm comprises an amino acid sequence comprising SEQ ID NO: 27 and the second arm comprises an amino acid sequence comprising SEQ ID NO: 28.

9. The monoclonal antibody of claim 3, wherein the first and third polypeptide chain each comprises an amino acid sequence comprising SEQ ID NO: 5, 10, 16, 20, or 27 and the second and fourth polypeptide chain each comprises an amino acid sequence comprising SEQ ID NO: 11, 15, 21, 25, or 28.

10. The monoclonal antibody of claims 1-8, wherein the first and second variable heavy chain domains each comprises a CDR-H1, CDR-H2, and CDR-H3 domain, and the first and second variable light chain domains each comprises a CDR-L1, CDR-L2, and CDR-L3 domain.

11. The monoclonal antibody of 10, wherein the CDR-H1, CDR-H2, and CDR-H3 of the first heavy chain domains and the CDR-H1, CDR-H2, and CDR-H3 of the second heavy chain domains comprises an amino acid sequence comprising SEQ ID NO: 6, 7, and 8, respectively, and wherein the CDR-L1, CDR-L2, and CDR-L3 of the first light chain domains and the CDR-L1, CDR-L2, and CDR-L3 of the second light chain domains comprises an amino acid sequence comprising SEQ ID NO: 12, 13, and 14, respectively.

12. The monoclonal antibody of claims 3-11, wherein the first and second polypeptide chains are linked by one or more covalent disulfide bonds and the third and fourth polypeptide chains are linked by one or more covalent disulfide bonds.

13. The monoclonal antibody of claims 3-12, wherein the first and third polypeptide chains are linked by one or more covalent disulfide bonds.

14. A scFv comprising a polypeptide comprising a variable heavy chain domain and a variable light chain domain, wherein the variable heavy chain domain and variable light chain domain form a binding site to an extracellular portion of human PAG.

15. The scFv of claim 14, further comprising a linker between the variable heavy chain domain and a variable light chain domain.

16. The scFv of claim 15, where the linker is a glycine serine linker.

17. The scFv of claim 15, where the linker is a glycine serine linker about 15 amino acids in length.

18. The scFv of claim 15, where the linker comprises SEQ ID NO: 26.

19. The scFv claims 14-18, wherein the polypeptide further comprises an Fc domain.

20. The scFv of claims 14-19, wherein the variable heavy chain domain comprises an amino acid sequence comprising SEQ ID NO: 5, 16, or 27, and wherein the variable light chain domain comprises an amino acid sequence comprising SEQ ID NO: 11, 21, or 28.

21. The scFv of claims 14-19, wherein the variable heavy chain domain comprises an amino acid sequence comprising SEQ ID NO: 5 and the variable light chain domain comprises an amino acid sequence comprising SEQ ID NO: 11, the variable heavy chain domain comprises an amino acid sequence comprising SEQ ID NO: 16 and the variable light chain domain comprises an amino acid sequence comprising SEQ ID NO: 21, or the variable heavy chain domain comprises an amino acid sequence comprising SEQ ID NO: 27 and the variable light chain domain comprises an amino acid sequence comprising SEQ ID NO: 28.

22. The scFv of claims 14-19, wherein the variable heavy chain domain comprises a CDR-H1, CDR-H2, and CDR-H3 domain, and the variable light chain domain comprises a CDR-L1, CDR-L2, and CDR-L3 domain.

23. The scFv of claim 22, wherein the CDR-H1, CDR-H2, and CDR-H3 of the heavy chain domain comprises an amino acid sequence comprising SEQ ID NO: 6, 7, and 8, respectively, and wherein the CDR-L1, CDR-L2, and CDR-L3 of the light chain domain comprises an amino acid sequence comprising SEQ ID NO: 12, 13, and 14, respectively.

24. The scFv of claim 19, wherein the polypeptide further comprises a second variable heavy chain domain and a second variable light chain domain, wherein the second variable heavy chain domain and second variable light chain domain form a second binding site to an extracellular portion of human PAG.

25. The scFv of claim 24, further comprising a linker between the second variable heavy chain domain and the second variable light chain domain.

26. The scFv of claim 25, where the linker is a glycine serine linker.

27. The scFv of claim 25, where the linker is a glycine serine linker about 15 amino acids in length.

28. The scFv of claim 25, where the linker comprises SEQ ID NO: 26.

29. The scFv of claims 24-28, wherein the first and second PAG binding sites are the same.

30. The scFv of claims 24-28, wherein the first and second variable heavy chain domains comprise the same sequence and the first and second variable light chain domains comprise the same sequence.

31. The scFv of claims 24-28, wherein the first and second variable heavy chain domains each comprise an amino acid sequence comprising SEQ ID NO: 5, 16, or 27, and wherein the first and second variable light chain domains each comprise an amino acid sequence comprising SEQ ID NO: 11, 21, or 28.

32. The scFv of claims 24-28, wherein the first and second variable heavy chain domains each comprise an amino acid sequence comprising SEQ ID NO: 5 and the first and second variable light chain domains each comprise an amino acid sequence comprising SEQ ID NO: 11, the first and second variable heavy chain domains each comprise an amino acid sequence comprising SEQ ID NO: 16 and the first and second variable light chain domains each comprise an amino acid sequence comprising SEQ ID NO: 21, or the first and second variable heavy chain domains each comprise an amino acid sequence comprising SEQ ID NO: 27 and the first and second variable light chain domains each comprise an amino acid sequence comprising SEQ ID NO: 28.

33. The scFv of claims 24-28, wherein the first and second variable heavy chain domains each comprises a CDR-H1, CDR-H2, and CDR-H3 domain, and the first and second variable light chain domains each comprises a CDR-L1, CDR-L2, and CDR-L3 domain.

34. The scFv of claim 33, wherein the CDR-H1, CDR-H2, and CDR-H3 of the first heavy chain domains and the CDR-H1, CDR-H2, and CDR-H3 of the second heavy chain domains comprises an amino acid sequence comprising SEQ ID NO: 6, 7, and 8, respectively, and wherein the CDR-L1, CDR-L2, and CDR-L3 of the first light chain domains and the CDR-L1, CDR-L2, and CDR-L3 of the second light chain domains comprises an amino acid sequence comprising SEQ ID NO: 12, 13, and 14, respectively.

35. The scFv of claims 19-34, wherein the Fc domain of the polypeptide associates with the Fc domain of a second polypeptide, wherein the second polypeptide is identical to the first polypeptide.

36. The scFv of claim 35, wherein the Fc region of the first polypeptide comprises knob mutations and the Fc region of the second polypeptide comprise hole mutations, or vice versa.

37. The monoclonal antibody of claims 1-13 or scFv of claims 14-36, wherein the monoclonal antibody or scFv is capable of localizing a PAG protein away from an immune synapse.

38. The monoclonal antibody of claims 1-13 or scFv of claims 14-36, wherein the monoclonal antibody or scFv is capable of localizing a PD-1 protein away from an immune synapse.

39. The monoclonal antibody of claims 1-13 or scFv of claims 14-36, wherein the monoclonal antibody or scFv is capable of disrupting downstream signaling of a PD-1 mediated response in a T cell.

40. The monoclonal antibody of claims 1-13 or scFv of claims 14-36, wherein the monoclonal antibody or scFv is capable of disrupting downstream signaling of a PAG mediated response in a T cell.

41. The monoclonal antibody of claims 1-13 or scFv of claims 14-36, wherein the monoclonal antibody or scFv is capable of enhancing T cell function.

42. The monoclonal antibody of claims 1-13 or scFv of claims 14-36, wherein the PAG protein is located on a T cell, and wherein the monoclonal antibody or scFv is capable of preventing the phosphorylation of PAG protein downstream of PD-1 signaling.

43. The monoclonal antibody of claims 1-13 or scFv of claims 14-36, wherein the monoclonal antibody or scFv is capable of inducing a cytokine secretion in a T cell.

44. The monoclonal antibody or scFv of claim 20, wherein the cytokine secretion is a secretion of IL-2.

45. A pharmaceutical composition comprising: the monoclonal antibody of any of claims 1-21 or the scFv of claims 14-36; and a pharmaceutically acceptable carrier.

46. A method of preventing or treating cancer in a subject comprising administering to the subject an effective amount of the pharmaceutical composition of claim 45.

47. The method of claim 46, wherein the cancer is selected from colorectal cancer, lung cancer, bladder cancer, breast cancer, cervical cancer, kidney cancer, leukemia, Hodgkin lymphoma, non-Hodgkin lymphoma, prostate cancer, skin cancer (e.g., melanoma), head and neck cancer, endometrial cancer, colon cancer, rectal cancer, liver cancer, thyroids cancer, esophageal cancer, renal cell cancer, testicular cancers, and a combination thereof.

48. The method of claim 46, wherein the cancer is selected from colorectal cancer, colon adenocarcinoma, renal cell carcinoma, melanoma, acute myeloid leukemia, invasive breast cancer, cervical squamous cancer, and testicular cancer.

49. The method of claim 46, wherein the cancer is colorectal cancer.

50. The method of claim 46, wherein the cancer is melanoma.

51. The method of claim 46, wherein the administration of the pharmaceutical composition is capable of inhibiting tumor growth.

52. The method of claim 46, wherein the administration of the pharmaceutical composition is capable of increasing T cell infiltration in the tumor.

53. The method of claim 46, wherein the pharmaceutical composition is administered in combination with an immune checkpoint therapy.

54. The method of claim 53, wherein the immune checkpoint therapy is an anti-PD-1 antibody.

55. The method of claim 54, wherein the administration of the pharmaceutical composition in combination with the anti-PD-1 antibody enhances the anti-PD-1 response.

56. A kit for generating a monoclonal antibody or fragment thereof or an scFv, the kit comprising one or more vectors comprising a polynucleotide sequence encoding any of the monoclonal antibodies of any of claims 1-21 or any of the scFvs of claims 14-36.

57. A kit for generating a monoclonal antibody or fragment thereof, the kit comprising:
a first vector comprising a polynucleotide sequence encoding the first polypeptide chain of the monoclonal antibody of claims 3-11; and
a second vector comprising a polynucleotide sequence encoding the second polypeptide chain of the monoclonal antibody of claims 3-11.

58. The kit of claim 57, wherein the first vector and the second vector are the same vector.

59. The kit of claim 57, wherein the first vector and the second vector are two different vectors.

60. One or more host cells comprising: one or more vectors comprising a polynucleotide sequence encoding any of the monoclonal antibodies of any of claims 1-21 or any of the scFvs of any of claims 14-36.

61. One or more host cells comprising:
a first vector comprising a polynucleotide sequence encoding the first polypeptide chain of the monoclonal antibody of claims 3-11; and
a second vector comprising a polynucleotide sequence encoding the second polypeptide chain of the monoclonal antibody of claims 3-11.

62. The one or more host cells of claim 61, wherein the first vector and the second vector are the same vector.

63. The one or more host cells of claim 61, wherein the first vector and the second vector are two different vectors.

64. A method of making a monoclonal antibody or fragment thereof or scFv comprising:
culturing the one or more host cells of claim 60 under conditions suitable for an expression of the one or more vectors; and
recovering the monoclonal antibody or fragment thereof or scFv.

65. A method of making a monoclonal antibody or fragment thereof comprising:
culturing the one or more host cells of any of claims 61-63 under conditions suitable for an expression of the first vector and the second vector; and
recovering the monoclonal antibody or fragment thereof.

66. A composition comprising: one or more vectors comprising a polynucleotide sequence encoding any of the monoclonal antibodies of any of claims 1-21 or any of the scFvs of any one of claims 14-36.

67. A composition comprising:
a first vector comprising a polynucleotide sequence encoding the first polypeptide chain of the monoclonal antibody of claims 3-11; and
a second vector comprising a polynucleotide sequence encoding the second polypeptide chain of the monoclonal antibody of claims 3-11.

68. The composition of claim 67, wherein the first vector and the second vector are the same vector.

69. The composition of claim 67, wherein the first vector and the second vector are two different vectors.

70. A means for binding an extracellular portion of a human PAG protein.

71. The means of claim 47, wherein the means for binding an extracellular portion of a human PAG protein comprises:
a first arm comprising a first variable heavy chain domain and a first variable light chain domain, wherein a portion of the first arm is capable of binding to the extracellular portion of the human PAG protein; and
a second arm comprising a second variable heavy chain domain and a second variable light chain domain, wherein a portion of the second arm is capable of binding to the extracellular portion of the human PAG protein,
wherein the first and second arms each further comprise a fragment, crystallizable (Fc) domain.

72. The means of claim 71, wherein the first and second arms each further comprise a CH1 domain, a hinge domain, and a CL domain.

73. The means of claims 71-72, wherein:
the first variable heavy chain domain of the first arm is encoded by a first polypeptide chain;
the first variable light chain domain of the first arm is encoded by a second polypeptide chain;
the second variable heavy chain domain of the second arm is encoded by a third polypeptide chain;
the second variable light chain domain of the second arm is encoded by a fourth polypeptide chain; and
the first variable heavy chain domain and first variable light chain domain form a first PAG binding site and wherein the second variable heavy chain domain and second variable light chain domain form a second PAG binding site.

74. The means of claim 73, wherein the first and second PAG binding sites are the same.

75. The means of claim 73, wherein the first and third polypeptide chain each further encode a hinge domain, a CH1 domain, and the Fc domain, and wherein the second and fourth polypeptide chain each further encode a CL domain.

76. The means of claim 73, wherein the first and third polypeptide chains comprise the same sequence and the second and fourth polypeptide chains comprise the same sequence.

77. The means of claims 71-76, wherein the first variable heavy chain domain comprises an amino acid sequence of the variable heavy chain portion of SEQ ID NO: 5, 16, or 27, wherein the first variable light chain domain comprises an amino acid sequence of the variable light chain portion of SEQ ID NO: 11, 21, or 28.

78. The means of claims 71-76, wherein the first arm comprises an amino acid sequence comprising SEQ ID NO: 5 and the second arm comprises an amino acid sequence comprising SEQ ID NO: 11, the first arm comprises an amino acid sequence comprising SEQ ID NO: 10 and the second arm comprises an amino acid sequence comprising SEQ ID NO: 15, the first arm comprises an amino acid sequence comprising SEQ ID NO: 16 and the second arm comprises an amino acid sequence comprising SEQ ID NO: 21, the first arm comprises an amino acid sequence comprising SEQ ID NO: 20 and the second arm comprises an amino acid sequence comprising SEQ ID NO: 25, or the first arm comprises an amino acid sequence comprising SEQ ID NO: 27 and the second arm comprises an amino acid sequence comprising SEQ ID NO: 28.

79. The means of claim 70, wherein the means comprises any one of the scFvs of claims 14-36.

80. The means of claims 70-79, wherein the means is capable of localizing a PD-1 protein away from an immune synapse.

81. The means of claims 70-79, wherein the means is capable of localizing a PAG protein away from an immune synapse.

82. The means of claims 70-79, wherein the means is capable of disrupting downstream signaling of a PD-1 mediated response in a T cell.

83. The means of claims 70-79, wherein the means is capable of disrupting downstream signaling of a PAG-mediated response in a T cell.

84. The means of claims 70-79, wherein the means is capable of enhancing T cell function.

85. The means of claims 70-79, wherein the PAG protein is located on a T cell, and wherein the means is capable of preventing the phosphorylation of PAG protein downstream of PD-1 signaling.

86. The means of claims 70-79, wherein the means is capable of inducing a cytokine secretion in a T cell.

87. The means of claim 86, wherein the cytokine secretion is a secretion of IL-2.

88. The method of any one of claims 46-55, wherein the subject is a human subject.
